SEQUENCE LISTING (108 sequences)

SEQ ID NO: 1 - DNA, 1345 bp, Ehrlichia sp.
ttgagcttgagattggttacgagcgcttcaagaccaagggtattagagatagtggtagtaaggaagatgaagctgatacagtatatctactagctaaggagttagcttatgatgttgttactggtcagactgataaccttgccgctgctcttgccaaaacctccggtaaggatattgttcagtttgctaaggcggtggagatttctcattccgagattgatggcaaggtttgtaagacgaagtcggcgggaactggaaaaaatccgtgtgatcatagccaaaagccgtgtagtacgaatgcgtattatgcgaggagaacgcagaagagtaggagttcgggaaaaacgtctttatgcggggacagtgggtatagcgggcaggagctaataacgggtgggcattatagcagtccaagcgtattccggaattttgtcaaagacacactacaaggaaatggtagtgagaactggcctacatctactggagaaggaagtgagagtaacgacaacgccatagccgttgctaaggacctagtaaatgaacttactcctgaagaacgaaccatagtggctgggttacttgctaaaattattgaaggaagcgaggttattgagattagggccatctcttcgacttcagttacaatgaatatttgctcagatatcacgataagtaatatcttaatgccgtatgtttgtgttggtccagggatgagctttgttagtgttgttgatggtcacactgctgcaaagtttgcatatcggttaaaggcaggtctgagttataaattttcgaaagaagttacagcttttgcaggtggtttttaccatcacgttataggagatggtgtttatgatgatctgccattgcggcatttatctgatgatattagtcctgtgaaacatgctaaggaaaccgccattgctagattcgtcatgaggtactttggcggggaatttggtgttaggctcgctttttaaggttgcgacctaaaagcacttagctcgccttcactcccccttaagcaatatgatgcacatttgttgccctacaaatctaatataaggtttgttgcctatactcgtgccgaattcggcacgaggaggaagctgaactcacccatcagtctctctcatccgttggccacctgctgtccccacccacccaccaaactggtgcttttaatggaatcagctttaaaaagaaaaaaatcctccaagtaacaaagcaccctataattattccgcagctccttgtcctcggtaattttaggcttgtgctgctatcattacacattacatggagttagggagtcatagctcttgtgtggccaatcagtgataca

SEQ ID NO: 2 - DNA, 1132 bp, Ehrlichia sp.
atttctatattggtttggattacagtccagcgtttagcaagataagagattttagtataagggagagtaacggagagacaaaggcagtatatccatacttaaaggatggaaagagtgtaaagctagagtcacacaagtttgactggaacacacctgatcctcggattgggtttaaggacaacatgcttgtagctatggaaggtagtgttggttatggtattggtggtgccagggttgagcttgagattggttacgagcgcttcaagaccaagggtattagagatagtggtagtaaggaagatgaagctgatacagtatatctactagctaaggagttagcttatgatgttgttactggacagactgataaccttgctgctgctcttgctaagacctcggggaaagacatcgttcagtttgctaaggcggttggggtttctcatcctagtattgatgggaaggtttgtaagacgaaggcggatagctcgaagaaatttccgttatatagtgacgaaacgcacacgaagggggcaaatgaggggagaacgtctttgtgcggtgacaatggtagttctacgataacaaccagtggtacgaatgtaagtgaaactgggcaggtttttagggattttatcagggcaacgctgaaagaggatggtagtaaaaactggccaacttcaagcggcacgggaactccaaaacctgtcacgaacgacaacgccaaagccgtagctaaagacctagtacaggagctaacccctgaagaaaaaaccatagtagcagggttactagctaagactattgaagggggtgaagttgttgagatcagggcggtttcttctacttccgtaatggtcaatgcttgttatgatcttcttagtgaaggtttaggtgttgttccttatgcttgtgttggtctcggtggtaacttcgtgggcgtggttgatggaattcattacacaaaccatctttaactctgaataccctagttaaggtaagtgaagtaactaggcaaattagtgctgcaccactcgtgaaacaaactacgatcagcgattcaccatacttagtaggtccgtacagtggctttacgctcttacccatcatgaaaaatacttgctatctaggaatc

SEQ ID NO: 3 - DNA, 554 bp, Ehrlichia sp.
ctactagctaaggagttagcttatgatgttgttactgggcagactgataaccttgctgctgctcttgccaagacttctggtaaagatattgttcagtttgctaagactcttaatatttctcactctaatatcgatgggaaggtttgtaggagggaaaagcatgggagtcaaggtttgactggaaccaaagcaggttcgtgtgatagtcagccacaaacggcgggtttcgattccatgaaacaaggtttgatggcagctttaggcgaacaaggcgctgaaaagtggcccaaaattaacaatggtggccacgcaacaatttatagtagtagcgcaggtccaggaaatgcgtatgctagagatgcatctactacggtagctacagacctaacaaagctcactactgaagaaaaaaccatagtagcagggttactagctagaactattgaagggggtgaagttgttgagattagggcagtttcttctacttctgtgatggttaatgcttgttatgatcttcttagtgaaggtttaggtgttgtaccttatgcttgtgt

SEQ ID NO: 4 - DNA, 559 bp, Ehrlichia sp.
atgctgtgaaaattactaactccactatcgatgggaaggtttgtaatggtagtagagagaaggggaatagtgctgggaacaacaacagtgctgtggctacctacgcgcagactcacacagcgaatacatcaacgtcacagtgtagcggtctagggaccactgttgtcaaacaaggttatggaagtttgaataagtttgttagcctgacgggggttggtgaaggtaaaaattggcctacaggtaagatacacgacggtagtagtggtgtcaaagatggtgaacagaacgggaatgccaaagccgtagctaaagacctagtagatcttaatcgtgacgaaaaaaccatagtagcaggattactagctaaaactattgaagggggtgaagttgttgagatcagggcggtttcttctacttctgtgatggttaatgcttgttatgatcttcttagtgaaggtttaggcgttgttccttacgcttgtgtcggtctcggaggtaacttcgtgggcgttgttgatgggcatatcactcctaagcttgcttatagattaaaggctgg

SEQ ID NO: 5 - DNA, 201 bp, Ehrlichia sp.
agcgcttcaagaccaagggtattagagatagtggtagtaaggaagatgaagctgatacagtatatctactagctaaggagttagcttatgatgttgttactggacagactgataaccttgccgctgctcttgctaaaacctcggggaaagactttgttcagtttgctaaggccgtggagatttctaattctacgattgggg

SEQ ID NO: 6 - DNA, 467 bp, Ehrlichia sp.
ggtatatcgatagcctacgtagtcactccttattattaaaaaggaagaccaagggtattagagatagtggaagtaaggaagatgaagcagatacagtatatctactagctaaggagttagcttatgatgttgttactgggcagactgataaccttgccgctgctcttgccaaaacctccggtaaggactttgttaaatttgccaatgctgttgttggaatttctcaccccgatgttaataagaaggtttgtgcgacgaggaaggacagtggtggtactagatatgcgaagtatgctgccacgactaataagagcagcaaccctgaaacctcactgtgtggagacgaaggtggctcgagcggcacgaataatacacaagagtttcttaaggaatttgtagcccaaaccctagtagaaaatgaaagtaaaaactggcctacttcaagcgggactgggttgaagactaac

SEQ ID NO: 7 - DNA, 530 bp, Ehrlichia sp.
aagatgaagctgatacagtatatctactggctaaggagttagcttatgatgttgttactggacagactgataagcttactgctgctcttgctaagacctccgggaaggactttgttcagtttgctaaggcggttggggtttctcatcctaatatcgatgggaaggtttgtaagactacgctagggcacacgagtgcggatagctacggtgtgtatggggagttaacaggccaggcgagtgcgagtgagacatcgttatgtggtggtaagggtaaaaatagtagtggtggtggagctgctcccgaagttttaagggactttgtaaagaaatctctgaaagatgggggccaaaactggccaacatctagggcgaccgagagttcacctaagactaaatctgaaactaacgacaatgcaaaagctgtcgctaaagacctagtagaccttaatcctgaagaaaaaaccatagtagcagggttactagctaaaactattgaaggtggggaagttgtagaaatcagagcagtttct

SEQ ID NO: 8 - protein, 325 aa, Ehrlichia sp.
ELEIGYERFKTKGIRDSGSKEDEADTVYLLAKELAYDVVTGQTDNLAAALAKTSGKDIVQFAKAVEISHSEIDGKVCKTKSAGTGKNPCDHSQKPCSTNAYYARRTQKSRSSGKTSLCGDSGYSGQELITGGHYSSPSVFRNFVKDTLQGNGSENWPTSTGEGSESNDNAIAVAKDLVNELTPEERTIVAGLLAKIIEGSEVIEIRAISSTSVTMNICSDITISNILMPYVCVGPGMSFVSVVDGHTAAKFAYRLKAGLSYKFSKEVTAFAGGFYHHVIGDGVYDDLPLRHLSDDISPVKHAKETAIARFVMRYFGGEFGVRLAF

SEQ ID NO: 9 - protein, 323 aa, Ehrlichia sp.
FYIGLDYSPAFSKIRDFSIRESNGETKAVYPYLKDGKSVKLESHKFDWNTPDPRIGFKDNMLVAMEGSVGYGIGGARVELEIGYERFKTKGIRDSGSKEDEADTVYLLAKELAYDVVTGQTDNLAAALAKTSGKDIVQFAKAVGVSHPSIDGKVCKTKADSSKKFPLYSDETHTKGANEGRTSLCGDNGSSTITTSGTNVSETGQVFRDFIRATLKEDGSKNWPTSSGTGTPKPVTNDNAKAVAKDLVQELTPEEKTIVAGLLAKTIEGGEVVEIRAVSSTSVMVNACYDLLSEGLGVVPYACVGLGGNFVGVVDGIHYTNHL

SEQ ID NO: 10 - protein, 185 aa, Ehrlichia sp.
LLAKELAYDVVTGQTDNLAAALAKTSGKDIVQFAKTLNISHSNIDGKVCRREKHGSQGLTGTKAGSCDSQPQTAGFDSMKQGLMAALGEQGAEKWPKINNGGHATIYSSSAGPGNAYARDASTTVATDLTKLTTEEKTIVAGLLARTIEGGEVVEIRAVSSTSVMVNACYDLLSEGLGVVPYACV

SEQ ID NO: 11 - protein, 185 aa, Ehrlichia sp.
AVKITNSTIDGKVCNGSREKGNSAGNNNSAVATYAQTHTANTSTSQCSGLGTTVVKQGYGSLNKFVSLTGVGEGKNWPTGKIHDGSSGVKDGEQNGNAKAVAKDLVDLNRDEKTIVAGLLAKTIEGGEVVEIRAVSSTSVMVNACYDLLSEGLGVVPYACVGLGGNFVGVVDGHITPKLAYRLKA

SEQ ID NO: 12 - protein, 66 aa, Ehrlichia sp.
RFKTKGIRDSGSKEDEADTVYLLAKELAYDVVTGQTDNLAAALAKTSGKDFVQFAKAVEISNSTIG

SEQ ID NO: 13 - protein, 155 aa, Ehrlichia sp.
YIDSLRSHSLLLKRKTKGIRDSGSKEDEADTVYLLAKELAYDVVTGQTDNLAAALAKTSGKDFVKFANAVVGISHPDVNKKVCATRKDSGGTRYAKYAATTNKSSNPETSLCGDEGGSSGTNNTQEFLKEFVAQTLVENESKNWPTSSGTGLKTN

SEQ ID NO: 14 - protein, 176 aa, Ehrlichia sp.
DEADTVYLLAKELAYDVVTGQTDKLTAALAKTSGKDFVQFAKAVGVSHPNIDGKVCKTTLGHTSADSYGVYGELTGQASASETSLCGGKGKNSSGGGAAPEVLRDFVKKSLKDGGQNWPTSRATESSPKTKSETNDNAKAVAKDLVDLNPEEKTIVAGLLAKTIEGGEVVEIRAVS

SEQ ID NO: 15 - DNA, 1185 bp, Ehrlichia sp.
gaaacagcattgctagatttcgttgaacaatttgctaatttgcaactaaagcactcatgataaagcttgatagtattttagaggatagtaggcaatatggtttaggggatttcttcgcatacttgttatcatcgtccttatttgtgcttagttggtcggatatttgtgcaagttgttgtaaaatatgcatattgtatgtataggtgtgcaagatatcatctctttaggtgtatcgtgtagcacttaaacaaatgctggtgaacgtagagggattaaaggaggatttgcgtatatgtatggtatagatatagagctaagtgattacagaattggtagtgaaaccatttccagtggagatgatggctactacgaaggatgtgcttgtgacaaagatgccagcactaatgcgtactcgtatgacaagtgtagggtagtacggggaacgtggagaccgagcgaactggttttatatgttggtgatgagcatgtggcatgtagagatgttgcttcgggtatgcatcatggtaatttgccaggggaaggtgtattttatagaggcagaagcgggcagagctgctactgctgaaggtggtgtttatactaccgttgtggaggcattatcgctggtgcaagaggaagagggtacaggtatgtacttgataaacgcaccagaaaaagcggtcgtaaggtttttcaagatagaaaagagtgcagcagaggaacctcaaacagtagatcctagtgtagttgagtcagcaacagggtcgggtgtagatacgcaagaagaacaagaaatagatcaagaagcaccagcaattgaagaagttgagacagaagagcaagaagttattctggaagaaggtactttgatagatcttgagcaacctgtagcgcaagtacctgtagtagctgaagcagaattacctggtgttgaagctgcagaagcgattgtaccatcactagaagaaaataagcttcaagaagtggtagttgctccagaagcgcaacaactagaatcagctcctgaagtttctgcgccagcacaacctgagtctacagttcttggtgttgctgaaggtgatctaaagtctgaagtatctgtagaagctaatgctgatgtacgcaaaaagaagtaatctctggtccacragagcaagaaattgcagaagcactagagggaactga

SEQ ID NO: 16 - DNA, 1131 bp, Ehrlichia sp.
ataaaggggctccagcaacgcagagagatgcttatggtaagacggctttacatatagcagctgctaatggtgacggtaagctatataagttaattgcgaaaaaatgcccagatagctgtcaagcactcctttctcatatgggagatacagcgttacatgaggctttatattctgataaggttacagaaaaatgctttttaaagatgcttaaagagtctcgaaagcatttgtcaaactcatctttcggagacttgcttaatactcctcaagaagcaaatggtgacacgttactgcatctggctgcatcgcgtggtttcggtaaagcatgtaaaatactactaaagtctggggcgtcagtatcagtcgtgaatgtagagggaaaaacaccggtagatgttgcggatccatcattgaaaactcgtccgtggttttttggaaagtccgttgtcacaatgatggctgaacgtgttcaagttcctgaagggggattcccaccatatctgccgcctgaaagtccaactccttctttaggatctatttcaagttttgagagtgtctctgcgctatcatccttgggtagtggcctagatactgcaggagctgaggagtctatctacgaagaaattaaggatacagcaaaaggtacaacggaagttgaaagcacatatacaactgtaggagctgaggagtctatctacgaagaaattaaggatacagcaaaaggtacaacggaagttgaaagcacatatacaactgtaggagctgaaggtccgagaacaccagaaggtgaagatctgtatgctactgtgggagctgcaattacttccgaggcgcaagcatcagatgcggcgtcatctaagggagaaaggccggaatccatttatgctgatccatttgatatagtgaaacctaggcaggaaaggcctgaatctatctatgctgacccatttgctgcggaacgaacatcttctggagtaacgacatttggccctaaggaagagccgatttatgcaacagtgaaaaagggtcctaagaagagtgatacttctcaaaaagaaggaacagcttctgaaaaagtcggctcaacaataactgtgattaagaagaaagtgaaacctcaggttccagcta

SEQ ID NO: 17 - DNA, 800 bp, Ehrlichia sp.
aatgcgctccacataactagcataacgttttcagcaacggcagatcttcatatataagcactgaacacctacgttccaagatcatgctcttcgcgcctgtttacttggtggctcagagtcatcatcactaggagttcgtggtctgtgagagctaacttgtgcttcttccagcgtataactagcacctcccaatcctgatgctgaaggttgatcccacgaataaggcataatcccttgatcctgaggtggcacatagggagcttgtgatcttcccattccagtactagtacctcctagcccagatgttgagaattggctagatggataaggaacattctctaggacacgtagtataatatgagggggggggggaacgagttgagctccctgtccggcagtacctcccaatcctgatgttgagggttgatcccatgatgttgagggttgatcccacgatgttgaaggttgtgcatacgaatagggcatcatccctggatcatgtggtggaatatgcgaagcttgttgacttcccattccagcggcacttcctaaccctgatgttgagggttgatcccacgatgttgaatgttgtgcatacgaatagggcatcatccctggatcatgtggtggaatatgcgaagcttgttgacttcccattccagcggcacttcctaaccctgatgttgagggttgatcccacgatgttgaaggttgtgcatacgaatagggcatcatccctggatcatgtggtggaatatgcgaagcttgttgacttcccgttccagcggcacttcctaacc

SEQ ID NO: 18 - DNA, 1011 bp, Ehrlichia sp.
aatgtatacagtctcagattcagaatctataacttctttcgttactccaccaatgttaatggcgaatatctcatcgactaagcgttcaggatacttgctatcattgtcggtagagccatctgacttttttaccgtgacattctttttaaaagaaactccatttacaacggacaattcagtgccattttgtagcttcgagcgcaactccacagcaaattcacgtattttcttcatacgtaatgcactcttccattcttcagtaagaatagacctgctttcttcaagtgtccttggtcttggaggcactacttcagtaacaagaacgccgaaataagcgtcaccattgctaaccagatgagacggttttcctacggcagatgaaaacgccaaagtagtaaaggcgtttataccaagctgcaacggaaagtctttcactaagttgccagatttatcgagcccatgcatatcaaaattcgtcaaaacaccactgatccgcgcaccaaacatatcctttagttcattcagcaatgccccgcggctgatcatatcgtttgcttttttcacattgctaactagcaactcacctgccttttgccttctaatatttgaagatatcttctctttcagcttttctaggtcttccttagtgatctcatgcttccttattaccttcatgatatgccagccgacaacgctacggaacatttcactgacttctccttcatttagtgcaaacaccacatttcgcacacctaccggaagaacatccttagagatattattgagtgcaatatcctctatggtgtagccagcatcactaaccaattcctcaaaagacttaccctcttggtaagctttgtaagctagctcagcttcatttttgtctgtaaatactaaatttagaacatctctttgatcatgtagttcactgtttttaatctcaacgtctaccttcttgatccgaaacaatgacatcagcaagcaagtcgtcttctgccatgattatatgat

SEQ ID NO: 19 - DNA, 513 bp, Ehrlichia sp.
gcaaatatttttcttggtgccgccctaaaagcctgaaaaatttaaagaaatgttactgctctagtcattcataaaatgcaaatagcctacagaaggagtatttactgctataggcttgaaagtgcaatcgttatttactattttttatacatatcgcagtacagagattttacgcgctacgcctgtgcatcatagccgtattgcatcaataaattgtcgttgctacgcgggaaagctgcttagcgcttgaccatttttcatacacattgtaccatcatagcgagtgtggtgctcatgagagtgcgtagtgttgccgccggtttctcatgttataatcttgctgccgttttgtgcagaaggaggagtagtctcgtttttttccaaaagacaatgtgctggagtgtcccggtgagcctcaaggttcttgtgggatttgtgtgggctgttgtataaataccacgttcgaagctgtcctagtgtattcagcatatgttgaggaagttgttgctatga

SEQ ID NO: 20 - DNA, 464 bp, Ehrlichia sp.
agtcattgagtcgagggtagtcttgtggatccctgataaatgttctaaaatttaaaacaacactagagttttgatcacatgttggttgtcagaaaaaaaatgtcaaaaaatttaccagggctttttgaaatgcctagattttccatttctcaatgaaacttgtttgatcatgactattccagctaatggagcagtgtgatgtagaggaaggagccactgagggtatgtggggtgttagactggatcatcattcttcaaggcgtgttccttggaatgcctgggaggagagcaattttctattaaaatttaattcgcctccttccaaatatggttccctggacgatttagcaaatagcattccttttttggagattcaaaaagcacattagcattgaggattgctacagtaaagaaatctgcctaactttgttttatccagtattgcctaaaattattggaccact

SEQ ID NO: 21 - DNA, 527 bp, Ehrlichia sp.
cctatggcagctctaaactcggcacgactggtttctacaagagattggtcgacattaaaccatgcgaaatcattgcgatcaattcttccttctttttcctgtatagcactacagacttcctctgcactagaagccactcgtgtcccgatgcgtacgtcacggatgcaaagccccaggtcttttacgctgccgggtgtgtctatatcttccacaacataatcaacgcaagcgtgaatatggataccagaaacagaggtaaccctgtatactaaatgctcttccaaaacatgttgattaacaggtaagcgcctagcactatcaccattatcagcaacaacgccttcatgcgcaacgtaatgagcagcgagctcaactggcagagatgacccactactgttactcaagatactagataagagtacccggagattttctgtgtttacaccagttttctccacaatatttgcagcatgcttcggctgtgaccttaagatttcacgtatttcatcggagtgttgtatgaaaat

SEQ ID NO: 22 - DNA, 464 bp, Ehrlichia sp.
ttcacctggccaaatcttattggatcttcaggacaaagaccaagaatctgcttctccaagaagcattctctgacccccacctacctatctgactcttagcttagattcctaatggtgtgagtgtgtcagagcctttacttagtctaagcgtaactgtaaaaacatcttttcaaaagtctctgcatgactgtctaggtctcacctatcacactgtaagcatctggaaaacaaagccactgagtcttccttttaccaaaaaggcctagccttgtttttgacaaatggcaagaacacattagatgtttgttgagagaacaaaaggagagaactcattatgaaactctggacaacatttatatacctctctacattttttgtgttggaggttagttttcttttctaataatttgatttctttggatacatcgaggcaatacacttaagaagcaagaagattgggggcc

SEQ ID NO: 23 - protein, 233 aa, Ehrlichia sp.
YGERGDRANWFYMLVMSMWHVEMLLRVCIMVICQGKVYFIEAEAGRAATAEGGVYTTVVEALSLVQEEEGTGMYLINAPEKAVVRFFKIEKSAAEEPQTVDPSVVESATGSGVDTQEEQEIDQEAPAIEEVETEEQEVILEEGTLIDLEQPVAQVPVVAEAELPGVEAAEAIVPSLEENKLQEVVVAPEAQQLESAPEVSAPAQPESTVLGVAEGDLKSEVSVEANADVRKKK

SEQ ID NO: 24 - protein, 376 aa, Ehrlichia sp.
KGAPATQRDAYGKTALHIAAANGDGKLYKLIAKKCPDSCQALLSHMGDTALHEALYSDKVTEKCFLKMLKESRKHLSNSSFGDLLNTPQEANGDTLLHLAASRGFGKACKILLKSGASVSVVNVEGKTPVDVADPSLKTRPWFFGKSVVTMMAERVQVPEGGFPPYLPPESPTPSLGSISSFESVSALSSLGSGLDTAGAEESIYEEIKDTAKGTTEVESTYTTVGAEESIYEEIKDTAKGTTEVESTYTTVGAEGPRTPEGEDLYATVGAAITSEAQASDAASSKGERPESIYADPFDIVKPRQERPESIYADPFAAERTSSGVTTFGPKEEPIYATVKKGPKKSDTSQKEGTASEKVGSTITVIKKKVKPQVPA

SEQ ID NO: 25 - protein, 148 aa, Ehrlichia sp.
YEGGGERVELPVRQYLPILMLRVDPMMLRVDPTMLKVVHTNRASSLDHVVEYAKLVDFPFQRHFLTLMLRVDPTMLKVVHTNRASSLDHVVEYAKLVDFPFQRHFLTLMLRVDPTMLKVVHTNRASSLDHVVEYAKLVDFPFQRHFLT

SEQ ID NO: 26 - protein, 89 aa, Ehrlichia sp.
YGSSKLGTTGFYKRLVDIKPCEIIAINSSFFFLYSTTDFLCTRSHSCPDAYVTDAKPQVFYAAGCVYIFHNIINASVNMDTRNRGNPVY

SEQ ID NO: 27 - protein, 238 aa, Ehrlichia sp.
LGSAAGTGSQQASHIPPHDPGMMPYSYAQPSTSWDQPSTSGLGSAAGMGSQQASHIPPHDPGMMPYSYAQPSTSWDQPSTSGLGSAAGMGSQQASHIPPHDPGMMPYSYAQPSTSWDQPSTSWDQPSTSGLGGTAGQGAQLVPPPPHIILRVLENVPYPSSQFSTSGLGGTSTGMGRSQAPYVPPQDQGIMPYSWDQPSASGLGGASYTLEEAQVSSHRPRTPSDDDSEPPSKQARRA

SEQ ID NO: 28 - protein, 334 aa, Ehrlichia sp.
SWQKTTCLLMSLFRIKKVDVEIKNSELHDQRDVLNLVFTDKNEAELAYKAYQEGKSFEELVSDAGYTIEDIALNNISKDVLPVGVRNVVFALNEGEVSEMFRSVVGWHIMKVIRKHEITKEDLEKLKEKISSNIRRQKAGELLVSNVKKANDMISRGALLNELKDMFGARISGVLTNFDMHGLDKSGNLVKDFPLQLGINAFTTLAFSSAVGKPSHLVSNGDAYFGVLVTEVVPPRPRTLEESRSILTEEWKSALRMKKIREFAVELRSKLQNGTELSVVNGVSFKKNVTVKKSDGSTDNDSKYPERLVDEIFAINIGGVTKEVIDSESETVYI

SEQ ID NO: 29 - protein, 175 aa, Ehrlichia sp.
IFIQHSDEIREILRSQPKHAANIVEKTGVNTENLRVLLSSILSNSSGSSLPVELAAHYVAHEGVVADNGDSARRLPVNQHVLEEHLVYRVTSVSGIHIHACVDYVVEDIDTPGSVKDLGLCIRDVRIGTRVASSAEEVCSAIQEKEGRIDRNDFAWFNVDQSLVETSRAEFRAAI

SEQ ID NO: 30 - protein, 41 aa, Ehrlichia sp.
  VARIANT (7)...(7): Xaa = Methionine or Threonine
LGSAAGXGSQQASHIPPHDPGMMPYSYAQPSTSWDQPSTSG

SEQ ID NO: 31 - DNA, 860 bp, Ehrlichia sp.
aaaagcttaaggaagatgtggcttctatgtcggatgaggctttgctgaagtttgccaataggctcagaagaggtgttcctatggctgctccggtgtttgagggtccgaaggatgcgcagatttcccggcttttggaattagcggatgttgatccgtctgggcaggtggatctttatgatgggcgttcagggcagaagtttgatcgcaaggtaactgttggatacatttacatgttgaagctccatcacttggtggatgacaagatacatgctaggtctgttggtccgtatggtctggttactcagcaacctcttggaggaaagtcgcactttggtgggcagagatttggggaaatggaatgctgggcattgcaggcctatggtgctgcttatactttgcaggaaatgctaactgtcaaatctgacgatatcgtaggtagggtaacaatctatgaatccataattaagggggatagcaacttcgagtgtggtattcctgagtcgtttaatgtcatggtcaaggagttacgctcgctgtgccttgatgttgttctaaagcaggataaagagtttactagtagcaaggtggagtagggatttacaattatgaagacgttggatttgtatggctataccagtatagcacagtcgttcgataacatttgcatatccatatctagtccacaaagtataagggctatgtcctatggagaaatcaaggatatctctactactatctatcgtacctttaaggtggagaagggggggctattctgtcctaagatctttggtccggttaatgatgacgagtgtctttgtggtaagtataggaaaaagcgctacaggggcattgtctgtgaa

SEQ ID NO: 32 - protein, 196 aa, Ehrlichia sp.
KLKEDVASMSDEALLKFANRLRRGVPMAAPVFEGPKDAQISRLLELADVDPSGQVDLYDGRSGQKFDRKVTVGYIYMLKLHHLVDDKIHARSVGPYGLVTQQPLGGKSHFGGQRFGEMECWALQAYGAAYTLQEMLTVKSDDIVGRVTIYESIIKGDSNFECGIPESFNVMVKELRSLCLDVVLKQDKEFTSSKVE

SEQ ID NO: 33 - protein, 89 aa, Ehrlichia sp.
GFTIMKTLDLYGYTSIAQSFDNICISISSPQSIRAMSYGEIKDISTTIYRTFKVEKGGLFCPKIFGPVNDDECLCGKYRKKRYRGIVCE

SEQ ID NO: 34 - DNA, 484 bp, Ehrlichia sp.
atcataagctttacatgtcctatccaggcgattatccctatccatagcatagtaacgccctgcaacagtagcaatttcggcatttaagtgctcaattttagcgttcagcataccgatatacttctcagcagaacgcggtggaacatccctaccatctagaattacatgtataaaaaccttgatgccaaatccggtgataacctcaataatggtttccatgtgcgcctgaagagaatgcactccaccatcagaaagcagaccaatcatgtggcataccccacccttcgcctgtatatcgcgcacaaagtccaacaatttaggattcttgtgaacctcattaatctcaagattaattctcaacagatcctgaagcactatcctgccgcatcctatacttatgtgccctacttctgaattcccgaactgacctgaaggcaatccgacatccgttccactagcagacaaactactcataggacagcat

SEQ ID NO: 35 - protein, 161 aa, Ehrlichia sp.
CCPMSSLSASGTDVGLPSGQFGNSEVGHISIGCGRIVLQDLLRINLEINEVHKNPKLLDFVRDIQAKGGVCHMIGLLSDGGVHSLQAHMETIIEVITGFGIKVFIHVILDGRDVPPRSAEKYIGMLNAKIEHLNAEIATVAGRYYAMDRDNRLDRTCKAYD

SEQ ID NO: 36 - DNA, 1039 bp, Ehrlichia sp.
ttaatcagagcggttgtgctagtcctttccgaaattcctgtgctgaatgcggagatttcaggcgatgatatagtctacagggactattgtaacattggagtcgcggtaggtaccgataaggggttagtggtgcctgttatcagaagagcggaaactatgtcacttgctgaaatggagcaagcacttgttgacttaagtacaaaagcaagaagtggcaagctctctgtttctgatatgtctggtgcaacctttactattaccaatggtggtgtgtatgggtcgctattgtctacccctataatcaaccctcctcaatctggaatcttgggtatgcatgctatacagcagcgtcctgtggcagtagatggtaaggtagagataaggcctatgatgtatttggcgctatcatatgatcatagaatagttgacgggcaaggtgctgtgacgtttttggtaagagtgaagcagtacatagaagatcctaacagattggctctaggaatttagggggtttttatggggcggggtacaataaccatccactccaaagaggattttgcctgtatgagaagggctgggatgcttgcagctaaggtgcttgattttataacgccgcatgttgttcctggtgtgactactaatgctctgaatgatctatgtcacgatttcatcatttctgccggggctattccagcgcctttgggctatagagggtatcctaagtctatttgtacttcgaagaattttgtggtttgccatggcattccagatgatattgcattaaaaaacggcgatatagttaacatagacgttactgtgatcctcgatggttggcacggggatactaataggatgtattgggttggtgataacgtctctattaaggctaagcgcatttgtgaggcaagttataaggcattgatggcggcgattggtgtaatacagccaggtaagaagctcaatagcatagggttagctatagaggaagaaatcagaggttatggatactccattgttagagattactgcggacatggga

SEQ ID NO: 37 - protein, 168 aa, Ehrlichia sp.
LIRAVVLVLSEIPVLNAEISGDDIVYRDYCNIGVAVGTDKGLVVPVIRRAETMSLAEMEQALVDLSTKARSGKLSVSDMSGATFTITNGGVYGSLLSTPIINPPQSGILGMHAIQQRPVAVDGKVEIRPMMYLALSYDHRIVDGQGAVTFLVRVKQYIEDPNRLALGI

SEQ ID NO: 38 - protein, 177 aa, Ehrlichia sp.
GVFMGRGTITIHSKEDFACMRRAGMLAAKVLDFITPHVVPGVTTNALNDLCHDFIISAGAIPAPLGYRGYPKSICTSKNFVVCHGIPDDIALKNGDIVNIDVTVILDGWHGDTNRMYWVGDNVSIKAKRICEASYKALMAAIGVIQPGKKLNSIGLAIEEEIRGYGYSIVRDYCGHG

SEQ ID NO: 39 - DNA, 2129 bp, Ehrlichia sp.
tttacctctttttgaagaaatcttaaagaaaaagcatggggcacggtccaacacatcgaaccttccccatacttttcacgagaaagatatcctaataacttagaacatcttcatcgtcaggatcctttaacggcaaagcagtcggaacatctactaactcttgctgcataccagcatcagcttctacagatacttcaaccttctcaacttcttcagttgcttgtgtctcttgatcagagattcctgcttcttgctgcataccagcatcagcttctacagatacttcagacttcagatcaccttcagtaacaccaagaactgtagactcaggttgtactggcgcagaaacttcaggagctgattctagttgttgcgcttctggagcaactaccacttcttgaagcttattttcttctagtgatggtacaatcgcttctgcagcttcaacaccaggtaattctgcttcagctactacaggtacttgcgctacaggttgctcaagatctatcaaagtaccttcttctagaataacttctggctcttccgtttttgtttctacagatacttcaaccttttcaacttcttcagttgcttgtgtctcttgatcagagattcctgcttcttgctgcataccagcatcagcttctacagatacttcagacttcagatcaccttcagtaacaccaagaactgtagactcaggttgtgctggtgcagaaacttcaggagctgattctagttgttgcgcttctggagcaactaccacttcttgaagcttattttcttctagtgatggtacaatcgcttctgcagcttcaacaccaggtaattctgcttcagctactacaggtacttgtgctacaggttgctcaagatctatcaaagtatcttcctttagaagaacttctgtttcttcttttacttctacaggagcttcagttccctctagtgcttctgcaatttcttgctcttgttgaccagagattacttctttttgcgctacatcagcattagcttctacagatacttcagactttagatcaccttcagcaacaccaagaactgtagactcaggttgtgctggcgcagaaacttcaggagctgattctagttgttgcgcttctggagcaactaccacttcttgaagcttattttcttctagtgatggtacaatcgcttctgcagcttcaacaccaggtaattctgcttcagctactacaggtacttgcgctacaggttgctcaagatctatcaaagtaccttcttccagaataacttcttgctcttctgtctcaacttcttcaattgctggtgcttcttgatctatttcttgttcttcttgcgtatctacacccgaccctgttgctgactcaactacactaggatctactgtttgaggttcctctgctgcactcttttctatcttgaaaaaccttacgaccgctttttctggtgcgtttatcaagtacatacctgtaccctcttcctcttgcaccagcgataatgcctccacaacggtagtataaacaccaccttcagcagtagcagctctgcccgcttctgcctctataaaatacaccttccctggcaaattaccatgatgcatacccgaagcaacatctctacatgccacatgctcatcaccaacatataaaaccagttcgctcggtctccacgttccccgtactaccctacacttgtcatacgagtacgcattagtgctggcatctttgtcacaagcacatccttcgtagtagccatcatctccactggaaatggtttcactaccaattctgtaatcacttagctctatatctataccatacatatacgcaaatcctcctttaatccctctacgttcaccagcatttgtttaagtgctacacgatacacctaaagagatgatatcttgcacacctatacatacaatatgcatattttacaacaacttgcacaaatatccgaccaactaagcacaaataaggacgatgataacaagtatgcgaagaaatcccctaaaccatattgcctactatcctctaaaatactatcaagctttatcatgagtgctttagttgcaaattagcaaattgttcaacgaaatctagcaatgctgtttcctcgtgccg

SEQ ID NO: 40 - DNA, 1919 bp, Ehrlichia sp.
atgctgtgaaaattactaactccactatcgatgggaaggtttgtaatggtagtagagagaaggggaatagtgctgggaacaacaacagtgctgtggctacctacgcgcagactcacacagcgaatacatcaacgtcacagtgtagcggtctagggaccactgttgtcaaacaaggttatggaagtttgaataagtttgttagcctgacgggggttggtgaaggtaaaaattggcctacaggtaagatacacgacggtagtagtggtgtcaaagatggtgaacagaacgggaatgccaaagccgtagctaaagacctagtagatcttaatcgtgacgaaaaaaccatagtagcaggattactagctaaaactattgaagggggtgaagttgttgagatcagggcggtttcttctacttctgtgatggttaatgcttgttatgatcttcttagtgaaggtttaggcgttgttccttacgcttgtgtcggtctcggaggtaacttcgtgggcgttgttgatgggcatatcactcctaagcttgcttatagattaaaggctggcttgagttatcagctctctcctgaaatctctgcttttgctgggggtttctaccatcgtgttgtgggagatggtgtttatgatgatctgccagctcaacgtcttgtagatgatactagtccggcgggccgtactaaggatactgctgttgctaacttctccatggcttatgtcggtggggaatttggtgttaggtttgctttttaaggtggtttgttggaagcggggtaagtcaaacttaccccgcttctattagggagttagtatatgagatctagaagtaagctattattaggaagcgtaatgatgtcgatggctatagtcatggctgggaatgatgtcagggctcatgatgacgttagcgctttggagactggtggtgcgggatatttctatgttggtttggattacagtccagcgtttagcaagataagagattttagtataagggagagtaacggagagactaaggcagtatatccatacttaaaggatggaaagagtgtaaagctagagtcacacaagtttgactggaacactcctgatcctcggattgggtttaaggacaacatgcttgtagctatggaaggcagtgttggttatggtattggtggtgccagggttgagcttgagattggttacgagcgcttcaagaccaagggtattagagatagtggtagtaaggaagatgaagctgatacagtatatctactagctaaggagttagcttatgatgttgttactggacagactgataaccttgctgctgctcttgccaagacctctggaaaagatatcgttcagtttgccaatgctgttaaaattactaactccgctatcgatgggaagatttgtaataggggtaaggctagtggcggcagcaaaggcctgtctagtagcaaagcaggttcatgtgatagcatagataagcagagtggaagcttggaacagagtttaacagcggctttaggtgataaaggtgctgaaaagtggcctaaaattaataatggcactagcgacacgacactgaatggaaacgacactagtagtacaccgtacactaaagatgcctctgctactgtagctaaagacctcgtagctcttaatcatgacgaaaaaaccatagtagcagggttactagctaaaactattgaagggggtgaggttgttgagattagggcggtttcttctacttctgtaatggtcaatgcttgttatgatcttcttagtgaaggtctaggcgttgttccttacgcttgtgtcggtcttggaggtaacttcgtgggcgttgttgatgggcatatcactcctaagcttgcttatagattaaaggctggcttgagttatcagctctctcctgaa

SEQ ID NO: 41 - DNA, 3073 bp, Ehrlichia sp.
tcccatgtccgcagtaatctctaacaatggagtatccataacctctgatttcttcctctatagctaaccctatgctattgagcttcttacctggctgtattacaccaatcgccgccatcaatgccttataacttgcctcacaaatgcgcttagccttaatagagacgttatcaccaacccaatacatcctattagtatccccgtgccaaccatcgaggatcacagtaacgtctatgttaactatatcgccgttttttaatgcaatatcatctggaatgccatggcaaaccacaaaattcttcgaagtacaaatagacttaggataccctctatagcccaaaggcgctggaatagccccggcagaaatgatgaaatcgtgacatagatcattcagagcattagtagtcacaccaggaacaacatgcggcgttataaaatcaagcaccttagctgcaagcatcccagcccttctcatacaggcaaaatcctctttggagtggatggttattgtaccccgccccataaaaaccccctaaattcctagagccaatctgttaggatcttctatgtactgcttcactcttaccaaaaacgtcacagcaccttgcccgtcaactattctatgatcatatgatagcgccaaatacatcataggccttatctctaccttaccatctactgccacaggacgctgctgtatagcatgcatacccaagattccagattgaggagggttgattataggggtagacaatagcgacccatacacaccaccattggtaatagtaaaggttgcaccagacatatcagaaacagagagcttgccacttcttgcttttgtacttaagtcaacaagtgcttgctccatttcagcaagtgacatagtttccgctcttctgataacaggcaccactaaccccttatcggtacctaccgcgactccaatgttacaatagtccctgtagactatatcatcgcctgaaatctccgcattcagcacaggaatttcggaaaggactagcacaaccgctctgataaagaaggacataaacccaagcttaacatcatacctcttcacaaaggcatctttgtacttagctctgagctccatcactttgctcatatcaacttcattaaaggtgctgagtgtagcagaggtattttgtgactccttaagcctagcagctataacttggcggattttgctcatcttcacgcgtctttcacccaccacgtcgccatggcaactcatcagatccttagacggctggctagcaactatcttcttgtcttgttcactcttagcactcatacccaaagctctagaagtaggagttgtgttgattcctgcaacaaaatcttctacagtaggagttactagacctttgccttcaataattgtcttttcctgcggtttttgagtgctcactgcctgtgcaacaacgggttgagcaagcacctcctccttgctctctggctccttattaacaccctctgcagtagcctcaccctgtggccgtatgatagccaagacctgcccttggtaatcacttcttcatctgcaactctcaactctgtgagaacaccagcaacaggggctgatatttcaagagaagtcttgtctgtttcaacaatgaagagcacatcttctgcagatacagtatctcccacctttttcattacccgaatcggagcttctagaatggattcgccaccaagattctcagccctaacttctacagcatcacccataaatacaaaccagaactaaaacaaaaaacacagattgaaaggcagtgtaatcaccaaaagacactaatgtcaaaccatagatgaataccttgttataagtatccacgcgataacgctatgtaattttcagcagatttttgtaggtataaaatctcctcttcagtcatcatacgtagaaattttgcaggcctacctgcccataactctccagattttacaatcttacccctagtgagcagtgaacctgcagctaacatgctgccctcttccatcactgcacgatccataacgattgatcccatacccacaaaggcgttattcccaagagtacaagcatgcaatatgcagctatggccaatagtaacgaatttacctattacagtatcaccatgcatgctatctgtatgtactactgtattatcttgaatgtttgtaccttcacccacttcaattttatccacatcgcccctgagtacggttccataccatatgctggcattcttacctatacaaacatctcctatgatacgggcataacctgcgataaatgcagtgctatctacagacggtgatactcctgcataaggcaccagaacttccctcataacttcacaacctccagtgttctttaaacggcacagcatgatagtgtttttagcacaccataacggagtacaccaccactcttaacagatttggctctggcacactagatgcacacatatcttgtataggacttatatattgttgttcatgaaacgtgcgtaatgctatgggagattactattcttatgtatgtaaattaagcaaatttagcacgtgctactgcacccagcatgttctcattttctttaaaaggcagaccttcctttttcgaaatagccttttctttaggaagcgtaatgatgtctatggctatagtcatggctgggaatgatgtcagggctcatgatgacgttagcgctttggagactggtggtgcgggatatttctatgttggtttggattacagtccagcgtttagcaagataagagattttagtataagggagagtaacggagagactaaggcagtatatccatacttaaaggatggaaagagtgtaaagctagagtctaacaagtttgactggaacactcctgatcctcggattgggtttaaggacaacatgcttgtagctatggaaggcagtgttggttatggtattggtggtgccagggttgagcttgagattggttacgagcgcttcaagaccaagggtattagagatagtggtagtaaggaagatgaagctgatacagtatatctactagctaagga

SEQ ID NO: 42 - DNA, 3786 bp, Ehrlichia sp.
aaaagcttaaggaagatgtggcttctatgtcggatgaggctttgctgaagtttgccaataggctcagaagaggtgttcctatggctgctccggtgtttgagggtccgaaggatgcgcagatttcccggcttttggaattagcggatgttgatccgtctgggcaggtggatctttatgatgggcgttcagggcagaagtttgatcgcaaggtaactgttggatacatttacatgttgaagctccatcacttggtggatgacaagatacatgctaggtctgttggtccgtatggtctggttactcagcaacctcttggaggaaagtcgcactttggtgggcagagatttggggaaatggaatgctgggcattgcaggcctatggtgctgcttatactttgcaggaaatgctaactgtcaaatctgacgatatcgtaggtagggtaacaatctatgaatccataattaagggggatagcaacttcgagtgtggtattcctgagtcgtttaatgtcatggtcaaggagttacgctcgctgtgccttgatgttgttctaaagcaggataaagagtttactagtagcaaggtggagtagggatttacaattatgaagacgttggatttgtatggctataccagtatagcacagtcgttcgataacatttgcatatccatatctagtccacaaagtataagggctatgtcctatggagaaatcaaggatatctctactactatctatcgtacctttaaggtggagaagggggggctattctgtcctaagatctttggtccggttaatgatgacgagtgtctttgtggtaagtataggaaaaagcgctacaggggcattgtctgtgagaaatgcggagtggaggtaacttcttctaaagttagaagagagagaatggggcacatagagttggtctcacctgttgctcatatttggtttcttaaatccctgccgtcacgtataggtgctctgctagacatgcctttaaaggctatagagaatatactatatagtggagattttgtagtaattgatccggtagctactccttttgctaagggggaagtaatcagtgaggtagtttataatcaggcgcgggatgcctatggtgaggatggattttttgcgctcactggtgttgaagctataaaggagttgctaactcgccttgatttggaggctatcagggctactttgaggaatgagcttgagtcaacttcttcggaaatgaagcgtaagaaggttgttaagaggctcaggcttgttgagaattttattaagtctggtaataggccggagtggatgatcttgactgtaattcctgttcttccaccggatttgaggccgttggtatcactggaaaatggtagacctgcggtatcagatttaaatcaccattacaggactataataaaccgtaataacagattggaaaagctactcaagctgaatcctcctgcgatcatgatacgcaatgaaaagaggatgttgcaagaagcggtagatgctctgtttgacagcagtcggcgtagttacgtttccagtagagttggaagcatgggctataagaagtctcttagcgacatgctaaagggtaagcagggtaggtttaggcagaacttgcttggtaaaagggttgactattctggtaggtcagtaatagttgtgggccctagtttgaagctgcatcagtgtggtttgcccaagaagatggctcttgagctgttcaagccgttcatttgttctaagctgaagatgtacggtattgctccgactgtgaagttggctaacaagatgattcagagtgagaagcctgatgtttgggatgttttggatgaagtgattaaagagcatcctattctccttaatagggctcctacactgcatagattgggtcttcaggcgtttgatcctgtattgatagaaggtaaggcaatacagttgcatccgttggtatgtagtgcgtttaatgccgatttcgatggtgatcagatggcggtacacgtgccattgtctcaagaggcgcagcttgaggcgcgcgtgttgatgatgtctacaaataacatcttgagtccttctaacggtaggccaattatagttccgtctaaggatatcgttcttgggatatactatttaacgttgttggaagaagatcctgaagtgcgtgaagtgcagacttttgcggagttcagccacgtggagtacgcattgcatgaggggattgtgcatacgtgctcaaggataaagtacagaatgcagaagagtgcagctgatggtactgtatctagcgaaatagttgagactacgcctggtaggttgatattgtggcagatattcccgcagcataaggatttgacttttgacttgatcaaccaagtgcttacggttaaggaaatcacctccattgtggatcttgtctatagaagttgtggtcagagggagacggtagagttctctgacaaactgatgtattggggattcaagtatgcttcgcaatcaggtatttcttttggttgtaaggatatgattattcctgatactaaggctgcgcacgttgaagatgctagcgaaaagatcagggaattctctatacagtatcaggatggtttgataaccaagagcgagcgctataacaaagtggttgatgagtggtctaagtgtaccgatttgattgctagggatatgatgaaggctatatctttatgtgatgagccagcgcgttcaggcgctcctgatacgtaaccttgtcgccaagtgcaacttttcctaaactaaagcctcaaatctttattatattctgttaatgactcagtggacttttggcagaaagagctagtttcctttggtacaaacacttttatagagggttctgattaatctatccgatggtctaaaatcaaaataacatatgcaatcgttggctgaaaaagctcacccgtggtgttataacaataattcctctccttgttttcatatataaccttttggaaacattcctgttggagccaaaatttctatattttggaaacttggcatatggatggatgatggctgaagtatgccatttattttccttttggggaggactagagaaagcagaatagttgttacactacttttgaaagtaaagtttgtaggacaacccagtttaatgtggaataaagccctgttctttagttttcatgtcataacacatattcatttctaaacatttttcctgaccacccaatttaaagtagttgacatccccagaagtcactttctctaacagaggtcaacacacttttctgtgtactgccagacagtaaacattttggactttgtatgttatatggtctctttctgttgcaactactgaactcttccattgtagcacgaaggcggctgcagacaatatgtaaacagatgagcatgactctgatccattacagctctatttatggacactgaaatttaaatttgctaaaattttcacatcacaaaatattatcctacttttgatatttttctaacacttaaaaaatgtaaaaaacaattcctaactcacagaccaaacacaaccaggcagtagacagaatttgaccagtgagctatcatttgagaccctcagttccacattacttttagagaggttttttaaatgtcacttcttagcatctaaacaaatctatttacatatttatattacttctatagtgtcatgtgctaaaatttaagctcttgtattagtccgttctcacactgctataaagacatacctgagactgggtttcac

SEQ ID NO: 43 - DNA, 3735 bp, Ehrlichia sp.
aatgcgctccacataactagcataacgttttcagcaacggcagatcttcatatataagcactgaacacctacgttccaagatcatgctcttcgcgcctgtttacttggtggctcagagtcatcatcactaggagttcgtggtctgtgagagctaacttgtgcttcttccagcgtataactagcacctcccaatcctgatgctgaaggttgatcccacgaataaggcataatcccttgatcctgaggtggcacatagggagcttgtgatcttcccattccagtactagtacctcctagcccagatgttgagaattggctagatggataaggaacattctctaggacacgtagtataatatgagggggggggggaacgagttgagctccctgtccggcagtacctcccaatcctgatgttgagggttgatcccatgatgttgagggttgatcccacgatgttgaaggttgtgcatacgaatagggcatcatccctggatcatgtggtggaatatgcgaagcttgttgacttcccattccagcggcacttcctaaccctgatgttgagggttgatcccacgatgttgaaggttgtgcatacgaatagggcatcatccctggatcatgtggtggaatatgcgaagcttgttgacttcccattccagcggcacttcctaaccctgatgttgagggttgatcccacgatgttgaaggttgtgcatacgaatagggcatcatccctggatcatgtggtggaatatgcgaagcttgttgacttcccgttccagcggcacttcctaaccctgatgttgagggttgatcccacaatgttgaaggttgtgcatacgaatagggcatcatccctggatcatgtggtggaatatgcgaagcttgttgacttcccgttccagcagtaccccccattcctgatgttgagggttgatcccacggcgcaccatagggtatgggtatacgctcaagaacacgtagtgggacactgatagcttgtgctccttccactccagcactagtactccctaatcctgatgtcgagggttgactaggtgcagcaccggtctgctcaacagcattgaaatatcttccgtatttcttgtcacaaatattcatcattactgaaagataccgcaatgctgtattgcgccacttgacttctatctgtggaattaatagcgcatcttccgtaatatgctcattgatctcctcatagacatggcacatgtctaaaaatgatttgcgagccctgtatgccccgagctcccttcttctgctatataaagcacacaaaatctggagacaatgcccaatcctacctgcaacaacatgatctacattaccggtggaagcgtatactctatacatcaagaacaaaccacctactgcatgcactaaagcaccaccccgatacctttctcgcttgagtcgtaaatcaaaactgtgaactcctaaaccttcaacatatgcctctaaatagtagagaaaatttgccatcgctcttctagagagtcctagacgcaggcgtgcactttcattattacgtaccatcgcttcacatgcagctgcactagtctcaatagcatcaataacactgtccaagcaagcctctgtacgatgacggaaaaaacgcggtgtattaggctcaactaactcagcaaccttactgcaaagctctatgttatgccgcactacgcgcaaaatcgcctttatattctctgtttcctcagaatccaaagaagaatttaagcatctacttaaggctgaaaattttacatagcagtatgcacttaaagctgtcactgtatgagatgcactaccatctctacgctcactactcactgcaccagtaaacctcgtggcaatagttctggcacagcagttcactatagcaataacattcactatgatagcacatgccttgcctatttgtaggtgtgccttacgcttaataaagtcttgatccatgaacagcggcacttctttgttgcactgcgccgtgatgcagtcctgcaacgcgtcgtacaaccgattgatcaaactatacaacacccccggttctgcgcttgaagcaccttctgcagcagttatacagctgttaatactgtctatcttatcagctgccgcaaacacgacatctacaccccggagcttgacaaacgtatcgcgcaattccagcatacattgacgtatagcctgcaggcatgcagcatatggcctggaattagtcattattgaattacatacagtttctttatattccgcagaagagcaaccactgtaggcatatccagacataactggagtagtgaatatacgaggcatatgcatctaattaaccactggaacaacttcacaccttgaaagtgtagcataccggtgtgacgcagctcaatattaaagattatgcacttcgtgatcgtctactaggaggctcaagttcatcatcactaggagtttgtgatctaggagagactacctgtgctccttccagcgtagaactagcacctcctaatcctgatgttgagggttgtgcatacgaataatcttgcaacggaccacaaggtgcctgagcttgcagtgctccctgtccagcaggattacctcccaatcccgatgttgagggttgactaggtgaagagggcatatgccctggatcatgaggtagcgtataggaagcttgtgatcctcctattccagccccagcacttcctagtctagatgttgagggttgactaggcgaaccctcagtctgcctaatattattgaaatatctctcgtacttcttttcccaaataccaatcattgccgaaagataccccaacatagcactacagaacccaacttctgtctggggatttaatagtagacctcgcgtaacgcattcctgaatctcatcatagacagtacacatgtccaaatataattcttgtgccgtatattctgaagctcccgctcttctgaccttatatttatagagagtaagcaacatttgaagacaatgctcaattttactcgcaacaacatgccctgtattacccgtggaagcatatactctgtgcattgagaataaactaccaattgcatacactaaagcttgcacatacttgtcatgcctgaaacttttaaaagcaacgctcagtcctaaacttttatatgtcttgaaatggtgtaaaaaacctgttctcgcttttttagcgagagctaggcggttctttgcactatcgttatcactcaccatctcttcgcattcagccgaggtagacccaactgcatcaagcatactgtttaagcaactcaccgtacgatcacggaaacaatatggaatctccggatcaactagctcagcaaccttattacaaagctctatgttatgcctcaccacacgtagaatagcctttctacgcttagtttcctcaggacccggagaataatttaaacatctgcttaaagctgaaaattttgcatttacgtatgcacttaaagccatgttggcatgatacgcactatgctcatcagcctcacctattgcactgtcagacgcctcggttaaggttgtgacaaagcagcttgccatggtaatagcattcaccaggatagcacataccttagcgatttgtaggtgtacttcacgcctcgtgaagtctggatccatgaaccgcggcacttctttgttgcactgcgccgtggcacagtcatgcagcatattatatgcactatggatta

SEQ ID NO: 44 - DNA, 2322 bp, Ehrlichia sp.
aatgtatacagtctcagattcagaatctataacttctttcgttactccaccaatgttaatggcgaatatctcatcgactaagcgttcaggatacttgctatcattgtcggtagagccatctgacttttttaccgtgacattctttttaaaagaaactccatttacaacggacaattcagtgccattttgtagcttcgagcgcaactccacagcaaattcacgtattttcttcatacgtaatgcactcttccattcttcagtaagaatagacctgctttcttcaagtgtccttggtcttggaggcactacttcagtaacaagaacgccgaaataagcgtcaccattgctaaccagatgagacggttttcctacggcagatgaaaacgccaaagtagtaaaggcgtttataccaagctgcaacggaaagtctttcactaagttgccagatttatcgagcccatgcatatcaaaattcgtcaaaacaccactgatccgcgcaccaaacatatcctttagttcattcagcaatgccccgcggctgatcatatcgtttgcttttttcacattgctaactagcaactcacctgccttttgccttctaatatttgaagatatcttctctttcagcttttctaggtcttccttagtgatctcatgcttccttattaccttcatgatatgccagccgacaacgctacggaacatttcactgacttctccttcatttagtgcaaacaccacatttcgcacacctaccggaagaacatccttagagatattattgagtgcaatatcctctatggtgtagccagcatcactaaccaattcctcaaaagacttaccctcttggtaagctttgtaagctagctcagcttcatttttgtctgtaaatactaaatttagaacatctctttgatcatgtagttcactgtttttaatctcaacgtctacctcttgatccgaaacaatgacatcagcaagcaagtcgtcttctgccatgattatataatcagcactgcgatattcagggaaatttagagaattcttgtactgctcctcaaacaatttttgcaattcatcatcagatatatcacttcctgaaatgtctacggcatcagaagatatttccactatgtctgccacacgatgctgcagcaatcccaacacaacatcttttgctaatgcatcataataaggaatatgtaattccgccctattagggaataaacactccattagaatagtagaaggtaaagcattgcgaattttattcacataggacgactcagtcattccgctgtcagccaatacggcttcatatctctcctggtcgaagacaccattagcatcctgaaatattcttatatttttgatcagactccgtaagctatttgagccaacacgtatgcctaagtcatgagcaaacttttcaacgaccatgtcggctatcatgttcttgaggacaacttccttaataccaaactgattaatttgagcatcagacaatttgtgttgtaacatcttctctagttctgccaactcgttgcggtacattatacggtaatcccgcaatggtagacatttattacccaacattgcaacgcactgtccgttgccagaattagacaacttacccattggtatcatgcttccaaaagtgacaaaagccatggcacctaaaaccgttgccatgaccacccaaacataaatcttccttgatcgcataacagaacgcccatagctggtcagattcccgaaggaatatagtaatcagaaaaaatctgcaagactttttctagttgtttatgggcaatattctgaattttgcatagtagccattacgtaatgtatggatagacccgtattaatttgtttcggtacgatatatgaagttctaaaaagctatagaaccttgccatgcaaagcttaagagcccttacccatcccatatacatccgtgttaatgaaagcaccattctgctgcttgtgcagaattctacataagcatctcgtgccgctcgtgccgaattcggcacgaggaattagatttaatagcagaagagcagaggcactgtggtgactgaagcagcaattaaagtaatgtggccacagctaagtaatatcagcagacactgaagtgggggaaggaaggaacagattgttacctgggcatgatcaaatttctggattcagaaaagtgtggatgaaatcctggctttattattgatcagtgctgtgtgatacagcacctagtcctcaaactctttcttcttaagcatccacacttgcaaaatgtgcaacttccaatatccatctctaagg

SEQ ID NO: 45 - DNA, 2373 bp, Ehrlichia sp.
gcaaatatttttcttggtgccgccctaaaagcctgaaaaatttaaagaaatgttactgctctagtcattcataaaatgcaaatagcctacagaaggagtatttactgctataggcttgaaagtgcaatcgttatttactattttttatacatatcgcagtacagagattttacgcgctacgcctgtgcatcatagccgtattgcatcaataaattgtcgttgctacgcgggaaagctgcttagcgcttgaccatttttcatacacattgtaccatcatagcgagtgtggtgctcatgagagtgcgtagtgttgccgccggtttctcatgttataatcttgctgccgttttgtgcagaaggaggagtagtctcgtttttttccaaaagacaatgtgctggagtgtcccggtgagcctcaaggttcttgtgggatttgtgtgggctgttgtataaataccacgttcgaagctgtcctagtgtaattcagcatatgttgaggaagttgttgctatgaggttgatggtatggcgaaaagattcttaaacgacacagaaaagaaattactatctctgctcaagtcggtaatgcagcattataagcctcgtaccggttttgtcagggctttgctaagtgccctgcgttctataagtgtagggaatccgagacaaacagcacatgatctatctgtgttggttacacaggatttccttgtcgaggttattggctctttcagtacgcaagctatcgctccttccttcctcaacatcatggccctggtagatgaggaggcattaaatcactacgaccgccctgggcgtgctccaatgtttgcagacatgttgaggtatgcgcaagagcaaattcgtagaggtaatctgcttcagcatagatggaatgaggagacatttgcatcttttgcggatagttacctcaggagaaggcacgagcgtgtcagtgcggagcatcttcgccaggcgatgcagatcttgcatgcaccggctagttatcgcgtcctgtctacaaattggtttttgctgcgtttgattgctgcagggtacgtgaggaatgcagttgatgtggtcgatgcggaaagtgcagggcttacttctcctcggagctccagtgagcgtactgctattgaatcgctcctgaaggattatgatgaagagggtctcagcgagatgctcgagaccgaaaaaggtgtcatgacgagcctcttcggtactgtgttactctcgtgccgaattcggcacgagttgaaaagcagcctttttaaggtagacatcctgtatatgatttaagtctcacctcccaatggaatcatgaaacagttagaaaaataatgaactacgtcttatataatctttatcgctactttaaaaatgagtaatatattcagatttagtagaaacatccctgaggaacaatttgttttcacaaattacattggttcctcacatgcaagattattaagcattaaggaggaggatattggacattgtataccctgtaggaatagttttttattttcagaaataagctcagcttactgattgatggcaaagatagttgatgataaaatagaaaaaaacaaagttactcttcttaattttgtactcttcttacctcctttcatttttaattggttataagtaggtgaaagttaaaacttggcaatgtttgctttaggagttattacaattactcaggttagtagtatagttatacggtcatctttagtaaaacatcattcggagtcatagtcacacttatgaatatcacagaatggatatgtgactttggggtttttttgtgggatattttttgagatatttaaggcagaagtgccacctttacttcatttatttttatccgccccccccccaccccaccgtttctcagaaaggataaggttttcacagtaccagagacatttatctactaaaactttgaactaattaaaatatatagggccgggtgcagtggctcacgcctgtaatcccagcactttgggaggccgaggcgggcggatcacgaggtccggagatggagaccatcctggctaacacggtgaaaccccgtctctactaaaaacacaaaaaattagccgggcgaggtggcgggcacctggggtcccagctactggggaggctgaggcagaagaatggcgtgaacccaggaggcggatcttgcagtgagccaagatcgcgccactgcactccagcctgggcgacagaacaagactccatctcaataaataaataaataaataaaatattatttaatttaagagagttgaaatcattgaattgattcatttaaacaaggtaatttgcaatgggtctatttttaggctattttctttatagtagt

SEQ ID NO: 46 - DNA, 7091 bp, Ehrlichia sp.
cctatggcagctctaaactcggcacgactggtttctacaagagattggtcgacattaaaccatgcgaaatcattgcgatcaattcttccttctttttcctgtatagcactacagacttcctctgcactagaagccactcgtgtcccgatgcgtacgtcacggatgcaaagccccaggtcttttacgctgccgggtgtgtctatatcttccacaacataatcaacgcaagcgtgaatatggataccagaaacagaggtaaccctgtatactaaatgctcttccaaaacatgttgattaacaggtaagcgcctagcactatcaccattatcagcaacaacgccttcatgcgcaacgtaatgagcagcgagctcaactggcagagatgacccactactgttactcaagatactagataagagtacccggagattttctgtgtttacaccagttttctccacaatatttgcagcatgcttcggctgtgaccttaagatttcacgtatttcatcggagtgttgtatgaaaataccacagtccccacgcacaggtacagagtgagatgcccagcgatggcgcttccccagatcttcccatagcgaaaggccgtgagctactatttcctcagcaagattgaaaatgtggcctccggcaaaatctgtatcttttgcactgccagcgaggaaatctctaagtgatataccgcctccaagtgtaagtacattgccaaatgtattcacagttaccgccacatgacggagaatagtggcgcatgcatcgtgcgcctgagaggccacaaaggacatgcagacccccattttggatacagcatccctgccatgagaaacagcgccctgctgtactacactagatttatcgtatcctaccagaccaacaacgcctcgtacaactactcggaatacaccgctcgcttcttgactgattactgtattacaaaaagaaagctctaggacttctagcggcataccgctaataacgctgtaagctcttaggatgcattcatcaatatcgcttacatcgtaaaaaaccctacgagccatgtaacgtgggttatgcctctgcagattacacgcgctgtacaatacatgagtaggcttctcagggactctcacatagtgttttgccagagctttgggaatattgtgccaagaacatacagatccaggctcgccttgcctaacgtcgcggcaatctctctcagtaagcacgagctttacttttttcacagctgtacggtaaacaccctccgcctttgtcgatggagcaatgtcatactctacccacatcttaactttggctatgggtacaccactgttgtcctgaatactaaatatgcatgattcgtgtactgtcagagcaccgttcttgtagctactaggtgctgaagccaataaagaatgcaccctggagaaagtagtataactctgaacttcaaatgtggtagagtcctcttctctgactattgtcatatcttcagacaccccatccaggcatccaagaacaaaattagttaaatcctcttcctggttttttcctggcaagctgttataggcaagtgcaagggcatgccacagctggaaaggtacttgttggaaggcagtactgttactcgctgtcttatgcagagctcttgctaataaatctggggaagttagattctcatgtatgagtgcaggaggtaccgcactgccctcacgtagagtaaacccctctgctaagagtatgaccattctgcgtcgtgcaggatgactgttccgatcacgacataaaaagaaatctatcgcgctaccaagcagtgcaacggacgctttcgatgggttttgcttaagcagcagagtcatgggtgcctcatcttagttacttctagtgacaaagcggtacttttattcctgtaaggacagaaaggcctgtttttttccagaaatctacgccttacatgtatggaaacctgcgcatccagctatagatatcgcaaggcatagtgtgcagaatacggagctgtagcaggcgctcttaccccccagcaaagtacgcaaacctagcgacgactcgttctcacacgttgtgaacatacgtagtaacacaccttgacgtacctagcctacaccactagacatatagtgtaaaacaaaaagtaccagatccccgtctcaggggttgtaaaagtagcacattggaaacggactgttaagtatttatattactacttaggttcagaataaacattcgaattgtaatgcaccataggttagtaatgcactatgagtgagaaattacgcgaattggtactgtgcgatgatcttgaaatttacagttgtagacacggcgcatgcggaagatataacctctcaaaccctgcagaggttttactaatcatatgttttgtctaatacctgcccacaaaaaacatatgaaagccttcgtagctcaggtcggttctctggctgttttcatctctaggttttaattcccaagaattcgacttttcgcgctacctaagcatttttaatcaccgttgactattagagacgatataataagctacattgattatctgaaatatgtgatccttctaaaaatctttaggtgctttagaagaagtacatattaccctctatggcaacaacattgataatttaggtgaagtgtcacagcgtttcattatgaaaaaaagggatacttatttatggggaatggcaccttatgcaatatgagccttagggattgccacagtgttttggtttcacagcatgagtaaggacgtggttttttagcaagtatttattgtgctatgtgtgtaaaaagtaacatatgaagatcgctaaagaattcacactagaaataagttgatacctgatgatgtagtataaaggttgagcaatagtctttttttgactgtaaatcccgcatgcagctttatgtgtgtttatcgcaaaaagtgggcgtttgttgcaataaaaattgaaatgccaactattattgcacataccgtgctcatacccttaatcttgtagatgcgctgtaatcacaattcgcatgtgcagcaaaactgtaatagatagcttagcacagggacgaataatccctagattctacgctgcgggctagtgctttttttagcatctatacgggagtatctttgatatgataaacacacaacagcatgatgctgtgcttatatagcattggtatatattctgcgatgcggactaatcaatgttgtaatcaagtaaaaaatgcttttttgaaccgtatattgttcgtaaggcatgtattactcagttgtcgtactacaaattcctcttcctctagagcatgcaagtatgaatacagcttatgtgtgcgatgcgtagattactaatgcatgattagtgtagggtatgctgtattttttgcatgcgttttagatatgttacgcaacacatgtttttcaaggacgctgtggctatcacggatatgatagccacaatgcgctgctcttaggtcaactaggatgggctgtgggtttatgcatattaagcagtggctcctgcattcaaagctattctttgttgtggttaacaatcaaaaatagagagtagtttgtttataagaagatatgcaaaaaacctttttatccacagtaagccccaggcgtatcgatgcacaaggatccaccatggctatgtcttaaggatgtacccagaatatgatcgtatctcattggctaagcagagcgtcctccagtttctgattctacagatagtacatcctgtaatgaagaaatggatccttcatcaagtgtcgttgatggagcatcatccggacagtactttgtagtagtgctctcggagttcagatcatcgcttgtacttacatcatcatatgacgaagaaacatcaatcgtagcatgttcgggttgaggctctgccagatgcacttcctgagagaggaggtcatgatataaatcccacagatagtgctgtttttaaccaggtccctgaaaaactcttctggagaaactggcagaggagccattgcgtactgcagtttggtaatattcatgcctatgcaagggatgcgttgaacgcgaacaagtgtaggatctggtacgcgcgtatcttgaggagtaaagactttccgtttatagaaccgatgcttcaatctgagtagaagacgtcctaacggaggacatactctaaacagtaatggtggtgaggtctttatattgcagtctggtggagtgatgattgtcaggtttaatgaacagttatcatagagaactcgtccctctccttgtatagagatctcgtatttcagtgctgtgtttactttgaacgcaggagtcttttctccctctgtagactgcggcactttcaggagaaagtccaaattctcgcagactgcaatacgctctggtgttattgcatctacctgttttatattgctacacgctgatacatagatgcgatttagtagatttagcgtggcacctgcatcgctaaagaagtattctttatccaaagcatgttttataggccaaattacatcgaaacatacccaggctgacagccctccttgatggcaatggcttgctatttcatcaagcagtctaatgtctgggacgaccccatgacgatcatctcggaacattttttgcagcatggctatcgcgagacttctttcacgatagcggcgcaaaaatacccctctacttactccatatgttctctgacatacaagattaaggttagtgatgctcgacgattttatgctcctttctagtcttgcaatatgagcacttacattttgtctagggtaaaatgttttattgatgcaccagtcacatctatgcatatcgattagaaactgatggccgtacaagttagacttgtttttatacgaatcgcaaagtgcgctgtggaaggaaaaccccgatgcaccttccagccattttttcttttgagaatactttaaacttacatctatagaagggcgatgatccttatgcttagctttactatccttacttgcgtcagagctattgtgtgtgcagatatgtactgaattagcctcatcttctgccttagagacagcactactagatgttgaaaaaattgagattatcctaaaaaacagtgctctcaaatagttcaggataccactgacagttcttctagatccattgtgagtattctttttacgcaacttaaacctccatgttacacaatatgcagctttgctattttcctttctcatgtggatgcgctaatctgcgtttgatcagtagtaacgacgcgcgctgtagtgtagttgttccaacaatgaacatgcaaaattgctgcaatacttaacttcctccttctgaaatgcatttcccacatttcaggcttttactatttcatgctttacatcgtgtagcgcatttttgaaaaaacaagatattagtacagcatttctggtaaaccagtaattgttcctattcaaggtctctgaatcatgacgaccactttctttgcggcaattgagaaattcctcacatatttgatatacaccgcactttttgtttttgctccatgaatggattaccggatccaagggcattgctatacttcactgtgcaacactactgtaagtgtcgttagcatatcatgaaattattaaataatatgtagaatatgttgtgcaaaagacgcttataacaacttaatagtgaatttcatgaaatttgtgagtagttttctatcggaatacgtgttttagcaacgctatagatggggtaagatcgcttttatgttcagaaattcgcaaccatactattttctctgtatgcgaagacatgtcttagcgtcaagccacatatgtggggtacttaagcgttgccttgcacgcaacagctccacattgcctggatttttcttaacatcagctaattatataccagactcacagatatactacgcgtaaccagtcatattatgcagcacctgtacatgttctctggggagttcctttatgaaacgagacattttcatggattggctccagttattgatttctctcattgcagcacatgatatgtatagctgctctctagctcttgttatgccaacataggctaagcgcctctcttcttccagagcgtttccagttatgtcattcatggatttttcgtgtgggaagactccttcctcccatccggggaggaaaaccaacgggaactccaacccctttgcggcatgtaatgtcataacgtgtacgtagttattgtcttcttctaaagaatcattttctgccactaagctaatgtgttctaaaaacttcgacacatcatcgaatcctgatacggctgagaagagttcctttatgttctctattcttgatagacctgattccccgtctttttttagagattctatatatccagagtcatgagcaatagcttttagtacattgacggatgaatctctacttaacatttctctccaatcatcaaactgcttgagaagatcttgcagaatgttggatgtattatcagatagtaatccatcttttatcattgagtgtccggcttcagttagggaaatactgtgctttctcccatatgcacgaagcttattgacagtagaagttccgagcttgcgtttgggcttatttataattttctcaaacgctatgtcgttattggggttgactactactttgagatatgcaacaagatcgcggatttctaccctatcatagaacttggttccgccgataattttgtaaggtataccatatcttacgaagaactcctcgaagactctagtctgaaagctggctcttactagaacagcagtttcactaaatttataatcgtaagagctcttaatatgctcactaatgtattgagcttcgagccgtccatcgaagaacttcattaaaccaactttttgtcctgcctgattgtgcgtccataatgtttttttaaggcgggatttattattatcaattatcgctgatgctgaggctaatatgttagacgttgacctataattacattccagccttattactttagcgtctgggaaatcatctgaaaatctgagtat

SEQ ID NO: 47 - DNA, 3947 bp, Ehrlichia sp.
ggtatatcgatagcctacgtagtcactccttattattaaaaaggaagaccaagggtattagagatagtggaagtaaggaagatgaagcagatacagtatatctactagctaaggagttagcttatgatgttgttactgggcagactgataaccttgccgctgctcttgccaaaacctccggtaaggactttgttaaatttgccaatgctgttgttggaatttctcaccccgatgttaataagaaggtttgtgcgacgaggaaggacagtggtggtactagatatgcgaagtatgctgccacgactaataagagcagcaaccctgaaacctcactgtgtggagacgaaggtggctcgagcggcacgaataatacacaagagtttcttaaggaatttgtagccaaaaccctagtagaaaatgaaagtaaaaactggcctacttcaagcgggactgggttgaagactaacgacaacgccaaagccgtagccacggacctagtagcgcttaatcgtgacgaaaaaaccatagtagctgggctactagctaaaactattgaagggggtgaggttgttgaaataagggcagtttcttctacttctgtgatggcgcttgaactccgggtatgctggtgattttgaggtattgggagttataccgcaagtatataacttaaatactgcatcgtaaggatatccttctgtttctgagacactggtaagtatgcccattacctatgaatctctatgtagatgtaataagagcatacacagtaactcttattattaaaaacaagaccaatggtataagggatagaagaagagtattattagagaggatgaagtagatacagtatatctactagctaaggagttagcttatgatgttgttactggacagactgataagcttactgctgctcttgccaaaacctccggtaaagacatcgttcagtttgctaaggcggttggggtttctcatcccagtattgatgggaaggtttgtaggacgaagcggaaggctggtgacagtagcggcacctatgccaagtatggggaagaaacggataataatactagcggtcaaagtacggttgcggtttgtggagagaaggctggacacaacgccaatgggtcgggtaccgtgcagtctttaaaagactttgtaagagagacgctaaaagcggatggtaataggaattggcctacttcaagggagaaatcgggaaatactaacacaaagcctcaacctaacgacaacgccaaagctgtagctaaagacctagtacaagagcttaatcatgatgaaaaaaccatagtagctgggttactagctaaaactattgaaggtggggaagtggttgagattagggcggtttcttctacttctgtgatggtcaatgcttgttatgatcttcttagtgaaggtttaggtgttgttccttatgcttgcgtcgggctcggtggtaacttcgtgggcgtggttgatgggcatatcacaatccgttgggcttcgaccctatatgctcacagcaagtcactaggcaaaattggagctgcatcactccgaaacagactacgatcagcgattctccatacctagtagatcagtacagtggctttatactcttacccagcatgaaatacttgctatctaagaatctcctctaaaactttccagaggttatctgtacttcgagaggaagctaatctgcgactaatacggatggtgtttataatatcactcctaaacttgcttataggttaaaagctgggttgagttatcagctttctcatgaaatctcggcttttgcgggtggcttctaccatcgtgttgttggtgatggtgtttatgatgatcttccggctcaactacctacaaattgataggtacactaaaagcccacgtaataactctcattattaaaatgaggaagatgaagcagatacagtatatctactagctaaggagttagcttatgatgttgttactgggcagactgataaccttgctgctgctcttgccaaaacttccggtaaagactttgttcagtttgcgaatgctgtgaaaatttctgcccctaatactcgtgccgaattcggcacgagcggcacgagctatatttaacttataagaaatcagcagactatttttcaaattgattgtacaatttaccttacctgggaatatatgtgagaaccctggcttctctaccttttaacaatatttgctattattatttttaaagtattagctattgtggttatgtggaattaaatatcaacttggtttcaatttgcattttcctaatgaggaatgctgttgactacgttttgcatgtgcttgtgggccatttatgtatcttcattacatttgttaagggatcgtgtgagacattcattcatttttattttattgtcattccattacttgttaactctttctactagtcttttaaaataatgtttaatttatcacctttttatttatggctttcttttcttggccttgttggacagatatttttcctaccccacatcatgaagacagtcccctatgttcttgtttgtttgataaaatacgtagactttaactcttgaatgagatgcataacttacctcaaattaagtttgtgaatgttagtaggtagagggcaacatacaaattgtatatgaatatattgttgttccatcatcattggtttaaaaaattcttaattctcctgatgaaattacttgggatgtctgtcaaataaatcttaaaatactttttgttaatttttattaagtagtgtactgaaattaaattggaactggttaaatctatagattgttaaattgaatatataaaggttaaattgaaattcattcaattcatgtacttcttaaatttctatcagctaacttttataatttttggtatagaaatcatacacaacataaaaaaatactaagtattttatctatttttgatacaaatgtaaattaaaatttaattttttactgctaatattacttatttaaaattttaactcttaatcattaaatatctctaatatcacatatatatttcaatgtatataattataaagtaacacttcttccttgtcaatttgtgtggcttgtactaaattgtattaatttttctttatttaagatgtctttatttcctctttattcttcaataatatgttctctggaatcaaaatcaagatttacatttcttttatttctacacttgagagatatggtgtcagttcttcctggtttccatgatttccatagttcccactgttttcatgaaatccactgttaagcaatttatcccctttatataaagtgtcatttttttgttgttactttctttgttgtatttagtttttagaaatttgattatgatatgttgtagtgtagatttcccaggtgttttcttgtttgatgttctctagtttggtggctaccttgttgaatctataggtttttttatttacacttaactaaatttgagaagttgtcagccattattttcttaaattacttttgacttttttagcctctactatttctatttctttttttgaggctctgatgacatggatatgaggtcttttgttttagttccacaactcgtgccgctcgtgccgaattcggcacgagaaaaggacaaatgttgtacagtttcacttacatgagatacctagcacaggccttttcatagggaaagtggaatagaggttaccagagctcagggcattgggaaatggggagtattgtttaatgggcacggagtttctgtttgagatgaggaaaaagttctggaaatgtgcagtattgtacaagctcacaaattgtactaagctcatcaatttaatgttaatgccactgaattgtctacttaaaaattgttaaaatgttaattttcatattgtgtatatttgaccacagtttaaa

SEQ ID NO: 48 - DNA, 5521 bp, Ehrlichia sp.
ttcacctggccaaatcttattggatcttcaggacaaagaccaagaatctgcttctccaagaagcattctctgacccccacctacctatctgactcttagcttagattcctaatggtgtgagtgtgtcagagcctttacttagtctaagcgtaactgtaaaaacatcttttcaaaagtctctgcatgactgtctaggtctcacctatcacactgtaagcatctggaaaacaaagccactgagtcttccttttaccaaaaaggcctagccttgtttttgacaaatggcaagaacacattagatgtttgttgagagaacaaaaggagagaactcattatgaaactctggacaacatttatatacctctctacattttttgtgttggaggttagttttcttttctaataatttgatttctttggatacatcgaggcaatacacttaagaagcaagaagattggggccagccttctagactgttcaaagggttacacccaacagaagggaaatattcccgagatgaccttggtgcctgttggggtgatcaagcccaacaccaggccgtcggggctacaaagtccagtggggtcaaaggaatgagaaaagacaagttaagagtgcataaagtgtatccagggggctaacgctagattggaggctgtgaaggcccggagctctgggagcccacactatttattgctggagtagaaaggtagcagtgcatcaagtgtagctgtgacagtttagcattttctttgacacatatagaatatgctctgctgcttgatataatggagagcatgtttatgagcctgggagagcaaccaacaagtctgtgcacattccagaggctacgaggggctttatgccctgagccctggattccatccaagccgcaaggggttttatgccctgggcttagatttgtggcgtggcagtgcagccttccaccctttggcacagagcttggtgttccaaaggccacgaggggttttagaccctggaccccggacatcctccaaggatcttttatattacgacaaacaagccagtcctgcctcagctcttctaccaacaggtacctttggccaaatgtctgaaatagggttacagattctataactgatggatctcctaacaggataattgagtgtcttatagggaagttgacatttttttggttactctactccaaggcattgaattgtttacagtttttatttgttcatggtggaaactgtggctgtatattatttcttattggtgtaggctagtatgataaactttgcttatcttttagtttgttatcaacccatagtagcacatcaaactgaatctacaaaaaaaactatggaaaacccttatgtatgtgtttcatgagcaaaattacctttgcttcaaattccaaccttggaaatgtttcttgagtttctacaggtagtctaataccagattctatgtaccttgttgtaacctcgtgccgaattcggcacgagctcgtgccgtgctgagtcattatttcctctcatagatatagtgctttctgaaggaggaatatcctaccaaaatttaactgacattgcagtaataataggccctggaagctttactgggttaagagtatctctggcaacagcacaaggttttgagcttgcttctagtgttgctgttcatgggatcagtcttcttgaactacaagcatattcaattttgtgtgcttctgaacaaactgaagaagatatagttgctgtgatagaatctacaaaagccgattttgtctattatcaaatgttcaataactccctcattcccctaacaggtgtgcatttagtgcctctaaatgaagtgcctcaaggcaaaatattgaagggctcccctgctatagctttggataccaagtctattgggttgtaccttatttataaactatcaaatcgacttccgaaaactacacttgcccccatttattcgcgcttttaccactagagtgtccatgataatttaactgataacatcaatcgggctagatatgtgtctagcttttgtgcgtaagctcttatggaaataagtgtgatattttgcgagcacatggtgatggagagctcatctaaggcagcctcagtaacatgccccgcgtctatgaattgtgattgtaatgcgtattaaggattccacaatttcctgtgacaaccactaaaagtagtctacaagctataaactcttaaatctatagattgctagggctgataaagaacctttagcattagaagcgtagagagacactgatgggttagaatttgatacaaaaacatgaccttattactacaatagtttacttgtgagcagtgcacaccaagaatataacattaagcttctgagaggatacactcactgagactctgtgagatctgacgtacccttacccaatctactacactctacctctggcaacgcattctacagagcacgttttagcgtgaaaatcttcacacgaagataccgttgtattgtggctccagttagcgtcactaagtattgagctagcagttccaccttgattaaaaggtactgcatcttatacagactttagcagtcccattacatactcaccttgatctagaaaacaatgatctagccgcacctaacatttctatcttcaaaaaaccacttatagcgtttttctctccaacttctaaaacatactctatatactttaaaggttttattgaggaaatcagaaaagatttttcaagtaacactgagctttcttttaaacatctggtgcagagatatgtactacacaaactgaaatataaacgttttggaaaatatctataaatatgaaacattaagttttaagcataatatgctttaaaactagcagaatatattgcaacacatattctatacattcttgcttgcattagaataaaaatagattgctcaaggaaactgctaggtatacatataccttttcaccaaattagcagtgtataccttctggaatactcataagcgtcttgtgaatacgatgtttttctacactgcaggtaagatgacgtttggcctatttttcgtatcagcagggctcaggtaaatgatgtatgtgcggtgttattatctatcaacaaatgcgtatggtgtatttttgatgccgaaaattgtctccatctcacaggcagcatatcttactcttgtaagcatataaaattttagttcacagtgttaagaaacactgttatttgatcccttgaaggtatgcttaaacggtttgaaaatgcacgtcctgcagtgtgtttgtaatacctgttctaacaaccaagagctttaagcatctcgaaaaagcttttaagaaattgatgcgtcccctagtagtgccgcggtaagcattattatgaacgctcaaaggtatagtattttggcatattgaatattacagtacagcatcaatatacagtttaaaactcaagtatcacatctcctactgctatcatctatgctggaaaaactcatttataccctgtgatgcgcttttaagagtgttacactgttaattctttcctctgtttaaatgttatgcagaacatgagtaataaaactaatagaagatatgtgagaagaggcattcagcccattacttactcatggattagataagaaactagagccacgtttgcttctgtttttcgtgacatgcttatgtagaattctgcacaagcagcagaatggtgctttcattaacacggatgtatatgggatgggtaagggctcttaagctttgcatggcaaggttctatagctttttagaacttcatatatcgtaccgaaacaaattaatacgggtctatccatacattacgtaatggctactatgcaaaattcagaatattgcccataaacaactagaaaaagtcttgcagattttttctgattactatattccttcgggaatctgaccagctatgggcgttctgttatgcgatcaaggaagatttatgtttgggtggtcatggcaacggttttaggtgccatggcttttgtcacttttggaagcatgataccaatgggtaagttgtctaattctggcaacggacagtgcgttgcaatgttgggtaataaatgtctaccattgcgggattaccgtataatgtaccgcaacgagttggcagaactagagaagatgttacaacacaaattgtctgatgctcaaattaatcagtttggtattaaggaagttgtcctcaagaacatgatagccgacatggtcgttgaaaagtttgctcatgacttaggcatacgtgttggctcaaatagcttacggagtctgatcaaaaatataagaatatttcaggatgctaatggtgtcttcgaccaggagagatatgaagccgtattggctgacagcggaatgactgagtcgtcctatgtgaataaaattcgcaatgctttaccttctactattctaatggagtgtttattccctaatagggcggaattacatattccttattatgatgcattagcaaaagatgttgtgttgggattgctgcagcatcgtgtggcagacatagtggaaatatcttctgatgccgtagacatttcaggaagtgatatatctgatgatgaattgcaaaaattgtttgaggagcagtacaagaattctctaaatttccctgaatatcgcagtgctgattatataatcatggcagaagacgacttgcttgctgatgtcattgtttcggatcaagaggtagacgttgagattaaaaacagtgaactacatgatcaaagagatgttctaaatttagtatttacagacaaaaatgaagctgagctagcttacaaagcttaccaagagggtaagtcttttgaggaattggttagtgatgctggctacaccatagaggatattgcactcaataatatctctaaggatgttcttccggtaggtgtgcgaaatgtggtgtttgcactaaatgaaggagaagtcagtgaaatgttccgtagcgttgtcggctggcatatcatgaaggtaataaggaagcatgagatcactaaggaagacctagaaaagctgaaagagaagatatcttcaaatattagaaggcaaaaggcaggtgagttgctagttagcaatgtgaaaaaagcaaacgatatgatcagccgcggggcattgctgaatgaactaaaggatatgtttggtgcgcggatcagtggtgttttgacgaattttgatatgcatgggctcgataaatctggcaacttagtgaaagactttccgttgcagcttggtataaacgcctttactactttggcgttttcatctgccgtaggaaaaccgtctcatctggttagcaatggtgacgcttatttcggcgttcttgttactgaagtagtgcctccaagaccaaggacacttgaagaaagcaggtctattcttactgaagaatggaagagtgcattacgtatgaagaaaatacgtgaatttgctgtggagttgcgctcgaagctacaaaatggcac

SEQ ID NO: 49 - DNA, 1938 bp, Ehrlichia sp.
ttgaggagtattaagcaagtctccgaaagatgagtttgacaaatgctttcgagactctttaagcatctttaaaaagcatttttctgtaaccttatcagaatataaagcctcatgtaacgctgtatctcccatatgagaaaggagtgcttgacagctatctgggcattttttcgcaatttacttatatagcttaccgtcaccattagcagctgctatatgtaaagccgtcttaccataagcatctctctgcgttgctggagcccctttatccaagagcaacctagcagtcttctggttgccagcagctgttgctaaatgcaaggctggagttccagtgtgatccgtagacgaaagatctgcacccctctgtaaaaggaaatttacaatcctattagcctctttaaggttacttgcctcatttgccacttgaactgcagcagctaaagggctcatagatccggtaggagtatttatatgtgccccagcttctacaacacgctttaaatgctttatagctttacccccctgaaagcaccctccttgtatacccacagaaatagctggttctggagacgcatttacatcagcactgtttttaattaacgtcttcactgcagcatattgaccactagttagtgcttcagcggtcaaagttgtcttttttccttcaggagttgtaatttcttcatttacactaatcacttcagtggtaataagatgcctcaatacatctgctgcaccttttcttactgcctcgacagcaacatgctgcgggtaaggctcatatctcattaacatgtcaagtgctggtagcgatacttttccaccacttgcttcacgaatcgcatatacacctggagtaggaacaccatcctttacaggaaacttagaataactactcttccttccaagagcctgctgcaatatctctaaatttccatcctttgctgcgtaatgtattatagttccaccatcatgtgaccgagcatctacgtccatgctattacagcgtaacatagtcttaacaccctcagtgttgccccctttatacgcagctaccacaggcgtttcacctgtcactggagatggtacattgattgatggaatattacgcacattctcaatcaacatctgcaatttaacgcttacgcctttatggcttggctcatcctcaactatcatgtgaataggcgctttgccattcggtgctaattgatttacaacagactcaggagtgcatcttaccacctgctcaaaaacccccactgttgatttttgtgctgcagcatgtataggtgcattacctgcaatatctaaattagtaaaaggttcctctccatacctatgatatgcttcctccaatacccttttcgcaagaggatcaaaatttggggtcccattagaagatacaaaatgcaccagcgttgatgcgtcctctggattaggacatgtaaagagagattttacttctgaagaagctgagccatacactttatctgcaatgttcatggccttctcgaagatcttctcagcctccggtatagccttctaatagcatactgtactgcactcatcccttttttatccgggaatattagtgcctctgcacactgcgattgccctcaatatttgacgacaccgcttcttgcatcttgtcaatgtatgataaaacatcccgccttggccattgctttgcaacaatgtggcaaacggtttcaccagcatcatttgcaacgctaatatcacttaaccttgagagaagatgctttactttctggtgatccatacgctccgtagcaatatgaagcggagtgtttccacccggtcccttagcattaacatctgctataagagctttgtcgcatagtacatcaagattgcctaaagcatttttgcctactgaagatgcagctgtatgtaatggcgtattaccatcta

SEQ ID NO: 50 - protein, 578 aa, Ehrlichia sp.
MYGIDIELSDYRIGSETISSGDDGYYEGCACDKDASTNAYSYDKCRVVRGTWRPSELVLYVGDEHVACRDVASGMHHGNLPGKVYFIEAEAGRAATAEGGVYTTVVEALSLVQEEEGTGMYLINAPEKAVVRFFKIEKSAAEEPQTVDPSVVESATGSGVDTQEEQEIDQEAPAIEEVETEEQEVILEEGTLIDLEQPVAQVPVVAEAELPGVEAAEAIVPSLEENKLQEVVVAPEAQQLESAPEVSAPAQPESTVLGVAEGDLKSEVSVEANADVAQKEVISGQQEQEIAEALEGTEAPVEVKEETEVLLKEDTLIDLEQPVAQVPVVAEAELPGVEAAEAIVPSLEENKLQEVVVAPEAQQLESAPEVSAPAQPESTVLGVTEGDLKSEVSVEADAGMQQEAGISDQETQATEEVEKVEVSVETKTEEPEVILEEGTLIDLEQPVAQVPVVAEAELPGVEAAEAIVPSLEENKLQEVVVAPEAQQLESAPEVSAPVQPESTVLGVTEGDLKSEVSVEADAGMQQEAGISDQETQATEEVEKVEVSVEADAGMQQELVDVPTALPLKDPDDEDVLSY

SEQ ID NO: 51 - protein, 125 aa, Ehrlichia sp.
  VARIANT (1)...(1): Xaa = Threonine or Lysine
XEEXEVXLXEXTLIDLEQPVAQVPVVAEAELPGVEAAEAIVPSLEENKLQEVVVAPEAQQLESAPEVSAPXQPESTVLGVXEGDLKSEVSVEAXAXXXQXXXISXXQEXXXXEXXEXXEXXVEXX

SEQ ID NO: 52 - protein, 253 aa, Ehrlichia sp.
AVKITNSTIDGKVCNGSREKGNSAGNNNSAVATYAQTHTANTSTSQCSGLGTTVVKQGYGSLNKFVSLTGVGEGKNWPTGKIHDGSSGVKDGEQNGNAKAVAKDLVDLNRDEKTIVAGLLAKTIEGGEVVEIRAVSSTSVMVNACYDLLSEGLGVVPYACVGLGGNFVGVVDGHITPKLAYRLKAGLSYQLSPEISAFAGGFYHRVVGDGVYDDLPAQRLVDDTSPAGRTKDTAVANFSMAYVGGEFGVRFAF

SEQ ID NO: 53 - protein, 366 aa, Ehrlichia sp.
YMRSRSKLLLGSVMMSMAIVMAGNDVRAHDDVSALETGGAGYFYVGLDYSPAFSKIRDFSIRESNGETKAVYPYLKDGKSVKLESHKFDWNTPDPRIGFKDNMLVAMEGSVGYGIGGARVELEIGYERFKTKGIRDSGSKEDEADTVYLLAKELAYDVVTGQTDNLAAALAKTSGKDIVQFANAVKITNSAIDGKICNRGKASGGSKGLSSSKAGSCDSIDKQSGSLEQSLTAALGDKGAEKWPKINNGTSDTTLNGNDTSSTPYTKDASATVAKDLVALNHDEKTIVAGLLAKTIEGGEVVEIRAVSSTSVMVNACYDLLSEGLGVVPYACVGLGGNFVGVVDGHITPKLAYRLKAGLSYQLSPE

SEQ ID NO: 54 - protein, 340 aa, Ehrlichia sp.
RSDYQGQVLAIIRPQGEATAEGVNKEPESKEEVLAQPVVAQAVSTQKPQEKTIIEGKGLVTPTVEDFVAGINTTPTSRALGMSAKSEQDKKIVASQPSKDLMSCHGDVVGERRVKMSKIRQVIAARLKESQNTSATLSTFNEVDMSKVMELRAKYKDAFVKRYDVKLGFMSFFIRAVVLVLSEIPVLNAEISGDDIVYRDYCNIGVAVGTDKGLVVPVIRRAETMSLAEMEQALVDLSTKARSGKLSVSDMSGATFTITNGGVYGSLLSTPIINPPQSGILGMHAIQQRPVAVDGKVEIRPMMYLALSYDHRIVDGQGAVTFLVRVKQYIEDPNRLALGI

SEQ ID NO: 55 - protein, 177 aa, Ehrlichia sp.; identical to SEQ ID NO: 38

SEQ ID NO: 56 - protein, 197 aa, Ehrlichia sp.
EWWCTPLWCAKNTIMLCRLKNTGGCEVMREVLVPYAGVSPSVDSTAFIAGYARIIGDVCIGKNASIWYGTVLRGDVDKIEVGEGTNIQDNTVVHTDSMHGDTVIGKFVTIGHSCILHACTLGNNAFVGMGSIVMDRAVMEEGSMLAAGSLLTRGKIVKSGELWAGRPAKFLRMMTEEEILYLQKSAENYIALSRGYL

SEQ ID NO: 57 - protein, 172 aa, Ehrlichia sp.
ANLARATAPSMFSFSLKGRPSFFEIAFSLGSVMMSMAIVMAGNDVRAHDDVSALETGGAGYFYVGLDYSPAFSKIRDFSIRESNGETKAVYPYLKDGKSVKLESNKFDWNTPDPRIGFKDNMLVAMEGSVGYGIGGARVELEIGYERFKTKGIRDSGSKEDEADTVYLLAKE

SEQ ID NO: 58 - protein, 196 aa, Ehrlichia sp.; identical to SEQ ID NO: 32

SEQ ID NO: 59 - protein, 719 aa, Ehrlichia sp.
GFTIMKTLDLYGYTSIAQSFDNICISISSPQSIRAMSYGEIKDISTTIYRTFKVEKGGLFCPKIFGPVNDDECLCGKYRKKRYRGIVCEKCGVEVTSSKVRRERMGHIELVSPVAHIWFLKSLPSRIGALLDMPLKAIENILYSGDFVVIDPVATPFAKGEVISEVVYNQARDAYGEDGFFALTGVEAIKELLTRLDLEAIRATLRNELESTSSEMKRKKVVKRLRLVENFIKSGNRPEWMILTVIPVLPPDLRPLVSLENGRPAVSDLNHHYRTIINRNNRLEKLLKLNPPAIMIRNEKRMLQEAVDALFDSSRRSYVSSRVGSMGYKKSLSDMLKGKQGRFRQNLLGKRVDYSGRSVIVVGPSLKLHQCGLPKKMALELFKPFICSKLKMYGIAPTVKLANKMIQSEKPDVWDVLDEVIKEHPILLNRAPTLHRLGLQAFDPVLIEGKAIQLHPLVCSAFNADFDGDQMAVHVPLSQEAQLEARVLMMSTNNILSPSNGRPIIVPSKDIVLGIYYLTLLEEDPEVREVQTFAEFSHVEYALHEGIVHTCSRIKYRMQKSAADGTVSSEIVETTPGRLILWQIFPQHKDLTFDLINQVLTVKEITSIVDLVYRSCGQRETVEFSDKLMYWGFKYASQSGISFGCKDMIIPDTKAAHVEDASEKIREFSIQYQDGLITKSERYNKVVDEWSKCTDLIARDMMKAISLCDEPARSGAPDT

SEQ ID NO: 60 - protein, 439 aa, Ehrlichia sp.
IHSAYNMLHDCATAQCNKEVPRFMDPDFTRREVHLQIAKVCAILVNAITMASCFVTTLTEASDSAIGEADEHSAYHANMALSAYVNAKFSALSRCLNYSPGPEETKRRKAILRVVRHNIELCNKVAELVDPEIPYCFRDRTVSCLNSMLDAVGSTSAECEEMVSDNDSAKNRLALAKKARTGFLHHFKTYKSLGLSVAFKSFRHDKYVQALVYAIGSLFSMHRVYASTGNTGHVVASKIEHCLQMLLTLYKYKVRRAGASEYTAQELYLDMCTVYDEIQECVTRGLLLNPQTEVGFCSAMLGYLSAMIGIWEKKYERYFNNIRQTEGSPSQPSTSRLGSAGAGIGGSQASYTLPHDPGHMPSSPSQPSTSGLGGNPAGQGALQAQAPCGPLQDYSYAQPSTSGLGGASSTLEGAQVVSPRSQTPSDDELEPPSRRSRSA

SEQ ID NO: 61 - protein, 752 aa, Ehrlichia sp.
MHMPRIFTTPVMSGYAYSGCSSAEYKETVCNSIMTNSRPYAACLQAIRQCMLELRDTFVKLRGVDVVFAAADKIDSINSCITAAEGASSAEPGVLYSLINRLYDALQDCITAQCNKEVPLFMDQDFIKRKAHLQIGKACAIIVNVIAIVNCCARTIATRFTGAVSSERRDGSASHTVTALSAYCYVKFSALSRCLNSSLDSEETENIKAILRVVRHNIELCSKVAELVEPNTPRFFRHRTEACLDSVIDAIETSAAACEAMVRNNESARLRLGLSRRAMANFLYYLEAYVEGLGVHSFDLRLKRERYRGGALVHAVGGLFLMYRVYASTGNVDHVVAGRIGHCLQILCALYSRRRELGAYRARKSFLDMCHVYEEINEHITEDALLIPQIEVKWRNTALRYLSVMMNICDKKYGRYFNAVEQTGAAPSQPSTSGLGSTSAGVEGAQAISVPLRVLERIPIPYGAPWDQPSTSGMGGTAGTGSQQASHIPPHDPGMMPYSYAQPSTLWDQPSTSGLGSAAGTGSQQASHIPPHDPGMMPYSYAQPSTSWDQPSTSGLGSAAGMGSQQASHIPPHDPGMMPYSYAQPSTSWDQPSTSGLGSAAGMGSQQASHIPPHDPGMMPYSYAQPSTSWDQPSTSWDQPSTSGLGGTAGQGAQLVPPPPHIILRVLENVPYPSSQFSTSGLGGTSTGMGRSQAPYVPPQDQGIMPYSWDQPSASGLGGASYTLEEAQVSSHRPRTPSDDDSEPPSKQARRA

SEQ ID NO: 62 - protein, 110 aa, Ehrlichia sp.
MYTVSDSESITSFVTPPMLMANISSTKRSGYLLSLSVEPSDFFTVTFFLKETPFTTDNSVPFCSFERNSTANSRIFFIRNALFHSSVRIDLLSSSVLGLGGTTSVTRTPK

SEQ ID NO: 63 - protein, 149 aa, Ehrlichia sp.
DGFPTADENAKVVKAFIPSCNGKSFTKLPDLSSPCISKFVKTPLIRAPNISFSSFSNAPRLIISFAFFTLLTSNSPAFCLLIFEDIFSFSFSRSSLVISCFLITFMICQPTTLRNISLTSPSFSANTTFRTPTGRTSLEILLSAISSMV

SEQ ID NO: 64 - protein, 590 aa, Ehrlichia sp.
LLYSFGNLTSYGRSVMRSRKIYVWVVMATVLGAMAFVTFGSMIPMGKLSNSGNGQCVAMLGNKCLPLRDYRIMYRNELAELEKMLQHKLSDAQINQFGIKEVVLKNMIADMVVEKFAHDLGIRVGSNSLRSLIKNIRIFQDANGVFDQERYEAVLADSGMTESSYVNKIRNALPSTILMECLFPNRAELHIPYYDALAKDVVLGLLQHRVADIVEISSDAVDISGSDISDDELQKLFEEQYKNSLNFPEYRSADYIIMAEDDLLADVIVSDQEVDVEIKNSELHDQRDVLNLVFTDKNEAELAYKAYQEGKSFEELVSDAGYTIEDIALNNISKDVLPVGVRNVVFALNEGEVSEMFRSVVGWHIMKVIRKHEITKEDLEKLKEKISSNIRRQKAGELLVSNVKKANDMISRGALLNELKDMFGARISGVLTNFDMHGLDKSGNLVKDFPLQLGINAFTTLAFSSAVGKPSHLVSNGDAYFGVLVTEVVPPRPRTLEESRSILTEEWKSALRMKKIREFAVELRSKLQNGTELSVVNGVSFKKNVTVKKSDGSTDNDSKYPERLVDEIFAINIGGVTKEVIDSESETVYI

SEQ ID NO: 65 - protein, 245 aa, Ehrlichia sp.
GSCCYEVDGMAKRFLNDTEKKLLSLLKSVMQHYKPRTGFVRALLSALRSISVGNPRQTAHDLSVLVTQDFLVEVIGSFSTQAIAPSFLNIMALVDEEALNHYDRPGRAPMFADMLRYAQEQIRRGNLLQHRWNEETFASFADSYLRRRHERVSAEHLRQAMQILHAPASYRVLSTNWFLLRLIAAGYVRNAVDVVDAESAGLTSPRSSSERTAIESLLKDYDEEGLSEMLETEKGVMTSLFGTVL

SEQ ID NO: 66 - protein, 456 aa, Ehrlichia sp.
KAIPEAEKIFEKAMNIADKVYGSASSEVKSLFTCPNPEDASTLVHFVSSNGTPNFDPLAKRVLEEAYHRYGEEPFTNLDIAGNAPIHAAAQKSTVGVFEQVVRCTPESVVNQLAPNGKAPIHMIVEDEPSHKGVSVKLQMLIENVRNIPSINVPSPVTGETPVVAAYKGGNTEGVKTMLRCNSMDVDARSHDGGTIIHYAAKDGNLEILQQALGRKSSYSKFPVKDGVPTPGVYAIREASGGKVSLPALDMLMRYEPYPQHVAVEAVRKGAADVLRHLITTEVISVNEEITTPEGKKTTLTAEALTSGQYAAVKTLIKNSADVNASPEPAISVGIQGGCFQGGKAIKHLKRVVEAGAHINTPTGSMSPLAAAVQVANEASNLKEANRIVNFLLQRGADLSSTDHTGTPALHLATAAGNQKTARLLLDKGAPATQRDAYGKTALHIAAANGDGKLYK

SEQ ID NO: 67 - protein, 113 aa, Ehrlichia sp.
DGNTPLHTAASSVGKNALGNLDVLCDKALIADVNAKGPGGNTPLHIATERMDHQKVKHLLSRLSDISVANDAGETVCHIVAKQWPRRDVLSYIDKMQEAVSSNIEGNRSVQRH

SEQ ID NO: 68 - protein, 623 aa, Ehrlichia sp.
DEAPMTLLLKQNPSKASVALLGSAIDFFLCRDRNSHPARRRMVILLAEGFTLREGSAVPPALIHENLTSPDLLARALHKTASNSTAFQQVPFQLWHALALAYNSLPGKNQEEDLTNFVLGCLDGVSEDMTIVREEDSTTFEVQSYTTFSRVHSLLASAPSSYKNGALTVHESCIFSIQDNSGVPIAKVKMWVEYDIAPSTKAEGVYRTAVKKVKLVLTERDCRDVRQGEPGSVCSWHNIPKALAKHYVRVPEKPTHVLYSACNLQRHNPRYMARRVFYDVSDIDECILRAYSVISGMPLEVLELSFCNTVISQEASGVFRVVVRGVVGLVGYDKSSVVQQGAVSHGRDAVSKMGVCMSFVASQAHDACATILRHVAVTVNTFGNVLTLGGGISLRDFLAGSAKDTDFAGGHIFNLAEEIVAHGLSLWEDLGKRHRWASHSVPVRGDCGIFIQHSDEIREILRSQPKHAANIVEKTGVNTENLRVLLSSILSNSSGSSLPVELAAHYVAHEGVVADNGDSARRLPVNQHVLEEHLVYRVTSVSGIHIHACVDYVVEDIDTPGSVKDLGLCIRDVRIGTRVASSAEEVCSAIQEKEGRIDRNDFAWFNVDQSLVETSRAEFRAAI

SEQ ID NO: 69 - protein, 464 aa, Ehrlichia sp.
RIHMRKENSKAAYCVTWRFKLRKKNTHNGSRRTVSGILNYLRALFFRIISIFSTSSSAVSKAEDEANSVHICTHNSSDASKDSKAKHKDHRPSIDVSLKYSQKKKWLEGASGFSFHSALCDSYKNKSNLYGHQFLIDMHRCDWCINKTFYPRQNVSAHIARLERSIKSSSITNLNLVCQRTYGVSRGVFLRRYRERSLAIAMLQKMFRDDRHGVVPDIRLLDEIASHCHQGGLSAWVCFDVIWPIKHALDKEYFFSDAGATLNLLNRIYVSACSNIKQVDAITPERIAVCENLDFLLKVPQSTEGEKTPAFKVNTALKYEISIQGEGRVLYDNCSLNLTIITPPDCNIKTSPPLLFRVCPPLGRLLLRLKHRFYKRKVFTPQDTRVPDPTLVRVQRIPCIGMNITKLQYAMAPLPVSPEEFFRDLVKNSTICGIYIMTSSLRKCIWQSLNPNMLRLMFLRHMMM

SEQ ID NO: 70 - protein, 378 aa, Ehrlichia sp.
ILRFSDDFPDAKVIRLECNYRSTSNILASASAIIDNNKSRLKKTLWTHNQAGQKVGLMKFFDGRLEAQYISEHIKSSYDYKFSETAVLVRASFQTRVFEEFFVRYGIPYKIIGGTKFYDRVEIRDLVAYLKVVVNPNNDIAFEKIINKPKRKLGTSTVNKLRAYGRKHSISLTEAGHSMIKDGLLSDNTSNILQDLLKQFDDWREMLSRDSSVNVLKAIAHDSGYIESLKKDGESGLSRIENIKELFSAVSGFDDVSKFLEHISLVAENDSLEEDNNYVHVMTLHAAKGLEFPLVFLPGWEEGVFPHEKSMNDITGNALEEERRLAYVGITRAREQLYISCAAMREINNWSQSMKMSRFIKELPREHVQVLHNMTGYA

SEQ ID NO: 71 - protein, 209 aa, Ehrlichia sp.
YIDSLRSHSLLLKRKTKGIRDSGSKEDEADTVYLLAKELAYDVVTGQTDNLAAALAKTSGKDFVKFANAVVGISHPDVNKKVCATRKDSGGTRYAKYAATTNKSSNPETSLCGDEGGSSGTNNTQEFLKEFVAKTLVENESKNWPTSSGTGLKTNDNAKAVATDLVALNRDEKTIVAGLLAKTIEGGEVVEIRAVSSTSVMALELRVCW

SEQ ID NO: 72 - protein, 261 aa, Ehrlichia sp.
KKSIIREDEVDTVYLLAKELAYDVVTGQTDKLTAALAKTSGKDIVQFAKAVGVSHPSIDGKVCRTKRKAGDSSGTYAKYGEETDNNTSGQSTVAVCGEKAGHNANGSGTVQSLKDFVRETLKADGNRNWPTSREKSGNTNTKPQPNDNAKAVAKDLVQELNHDEKTIVAGLLAKTIEGGEVVEIRAVSSTSVMVNACYDLLSEGLGVVPYACVGLGGNFVGVVDGHITIRWASTLYAHSKSLGKIGAASLRNRLRSAILHT

SEQ ID NO: 73 - protein, 530 aa, Ehrlichia sp.
LLYSFGNLTSYGRSVMRSRKIYVWVVMATVLGAMAFVTFGSMIPMGKLSNSGNGQCVAMLGNKCLPLRDYRIMYRNELAELEKMLQHKLSDAQINQFGIKEVVLKNMIADMVVEKFAHDLGIRVGSNSLRSLIKNIRIFQDANGVFDQERYEAVLADSGMTESSYVNKIRNALPSTILMECLFPNRAELHIPYYDALAKDVVLGLLQHRVADIVEISSDAVDISGSDISDDELQKLFEEQYKNSLNFPEYRSADYIIMAEDDLLADVIVSDQEVDVEIKNSELHDQRDVLNLVFTDKNEAELAYKAYQEGKSFEELVSDAGYTIEDIALNNISKDVLPVGVRNVVFALNEGEVSEMFRSVVGWHIMKVIRKHEITKEDLEKLKEKISSNIRRQKAGELLVSNVKKANDMISRGALLNELKDMFGARISGVLTNFDMHGLDKSGNLVKDFPLQLGINAFTTLAFSSAVGKPSHLVSNGDAYFGVLVTEVVPPRPRTLEESRSILTEEWKSALRMKKIREFAVELRSKLQNG

SEQ ID NO: 74 - DNA, 25 bp, Artificial Sequence
  PCR primer used to prepare DNA for fusion construct
aaaggggctccagcaacgcagagag

SEQ ID NO: 75 - DNA, 32 bp, Artificial Sequence
  PCR primer used to prepare DNA for fusion construct
catagaattcgatcgatcgagtagctggaacc

SEQ ID NO: 76 - DNA, 28 bp, Artificial Sequence
  PCR primer used to prepare DNA for fusion construct
caccgtcgatcgttctatattggtttgg

SEQ ID NO: 77 - DNA, 32 bp, Artificial Sequence
  PCR primer used to prepare DNA for fusion construct
cttgactcgagttaaagatggtttgtgtaatg

SEQ ID NO: 78 - DNA, 29 bp, Artificial Sequence
  PCR primer used to prepare DNA for fusion construct
cttatcgatcggagcttgagattggttac

SEQ ID NO: 79 - DNA, 31 bp, Artificial Sequence
  PCR primer used to prepare DNA for fusion construct
caatgcgaattcattaaaaagcgagcctaac

SEQ ID NO: 80 - DNA, 33 bp, Artificial Sequence
  PCR primer used to prepare DNA for fusion construct
ctacatcacgtgttctatattggtttggattac

SEQ ID NO: 81 - DNA, 34 bp, Artificial Sequence
  PCR primer used to prepare DNA for fusion construct
ggttaactcgagtactaagatggtttgtgtaatg

SEQ ID NO: 82 - DNA, 27 bp, Artificial Sequence
  PCR primer used to prepare DNA for fusion construct
gagcttgagattggttacgagcgcttc

SEQ ID NO: 83 - DNA, 32 bp, Artificial Sequence
  PCR primer used to prepare DNA for fusion construct
caattactcgagaattcattaaaaagcgagcc

SEQ ID NO: 84 - DNA, 1980 bp, Artificial Sequence
  DNA fusion construct containing HGE-3 and HGE-1 antigens
atgcagcatcaccaccatcaccacgtgttctatattggtttggattacagtccagcgtttagcaagataagagattttagtataagggagagtaacggagagacaaaggcagtatatccatacttaaaggatggaaagagtgtaaagctagagtcacacaagtttgactggaacacacctgatcctcggattgggtttaaggacaacatgcttgtagctatggaaggtagtgttggttatggtattggtggtgccagggttgagcttgagattggttacgagcgcttcaagaccaagggtattagagatagtggtagtaaggaagatgaagctgatacagtatatctactagctaaggagttagcttatgatgttgttactggacagactgataaccttgctgctgctcttgctaagacctcggggaaagacatcgttcagtttgctaaggcggttggggtttctcatcctagtattgatgggaaggtttgtaagacgaaggcggatagctcgaagaaatttccgttatatagtgacgaaacgcacacgaagggggcaaatgaggggagaacgtctttgtgcggtgacaatggtagttctacgataacaaccagtggtacgaatgtaagtgaaactgggcaggtttttagggattttatcagggcaacgctgaaagaggatggtagtaaaaactggccaacttcaagcggcacgggaactccaaaacctgtcacgaacgacaacgccaaagccgtagctaaagacctagtacaggagctaacccctgaagaaaaaaccatagtagcagggttactagctaagactattgaagggggtgaagttgttgagatcagggcggtttcttctacttccgtaatggtcaatgcttgttatgatcttcttagtgaaggtttaggtgttgttccttatgcttgtgttggtctcggtggtaacttcgtgggcgtggttgatggaattcattacacaaaccatcttagtgagcttgagattggttacgagcgcttcaagaccaagggtattagagatagtggtagtaaggaagatgaagctgatacagtatatctactagctaaggagttagcttatgatgttgttactggtcagactgataaccttgccgctgctcttgccaaaacctccggtaaggatattgttcagtttgctaaggcggtggagatttctcattccgagattgatggcaaggtttgtaagacgaagtcggcgggaactggaaaaaatccgtgtgatcatagccaaaagccgtgtagtacgaatgcgtattatgcgaggagaacgcagaagagtaggagttcgggaaaaacgtctttatgcggggacagtgggtatagcgggcaggagctaataacgggtgggcattatagcagtccaagcgtattccggaattttgtcaaagacacactacaaggaaatggtagtgagaactggcctacatctactggagaaggaagtgagagtaacgacaacgccatagccgttgctaaggacctagtaaatgaacttactcctgaagaacgaaccatagtggctgggttacttgctaaaattattgaaggaagcgaggttattgagattagggccatctcttcgacttcagttacaatgaatatttgctcagatatcacgataagtaatatcttaatgccgtatgtttgtgttggtccagggatgagctttgttagtgttgttgatggtcacactgctgcaaagtttgcatatcggttaaaggcaggtctgagttataaattttcgaaagaagttacagcttttgcaggtggtttttaccatcacgttataggagatggtgtttatgatgatctgccattgcggcatttatctgatgatattagtcctgtgaaacatgctaaggaaaccgccattgctagattcgtcatgaggtactttggcggggaatttggtgttaggctcgctttttaatga

SEQ ID NO: 85 - protein, 658 aa, Artificial Sequence
  Amino acid sequence of fusion protein containing HGE-3 and HGE-1 antigens
MQHHHHHHVFYIGLDYSPAFSKIRDFSIRESNGETKAVYPYLKDGKSVKLESHKFDWNTPDPRIGFKDNMLVAMEGSVGYGIGGARVELEIGYERFKTKGIRDSGSKEDEADTVYLLAKELAYDVVTGQTDNLAAALAKTSGKDIVQFAKAVGVSHPSIDGKVCKTKADSSKKFPLYSDETHTKGANEGRTSLCGDNGSSTITTSGTNVSETGQVFRDFIRATLKEDGSKNWPTSSGTGTPKPVTNDNAKAVAKDLVQELTPEEKTIVAGLLAKTIEGGEVVEIRAVSSTSVMVNACYDLLSEGLGVVPYACVGLGGNFVGVVDGIHYTNHLSELEIGYERFKTKGIRDSGSKEDEADTVYLLAKELAYDVVTGQTDNLAAALAKTSGKDIVQFAKAVEISHSEIDGKVCKTKSAGTGKNPCDHSQKPCSTNAYYARRTQKSRSSGKTSLCGDSGYSGQELITGGHYSSPSVFRNFVKDTLQGNGSENWPTSTGEGSESNDNAIAVAKDLVNELTPEERTIVAGLLAKIIEGSEVIEIRAISSTSVTMNICSDITISNILMPYVCVGPGMSFVSVVDGHTAAKFAYRLKAGLSYKFSKEVTAFAGGFYHHVIGDGVYDDLPLRHLSDDISPVKHAKETAIARFVMRYFGGEFGVRLAF

SEQ ID NO: 86 - DNA, 3300 bp, Ehrlichia (HGE)
taaaataatctgccccctttagagcgttatgtactctaaaaggggtattattaaagtggcgagatcatcgcctaaatactcagaagcgcgaattatattgatcaaagtacctcagcgatttttcggtataattctacctaccgcgacctccttttacagacttagggccttcactttgaggagcttctggttgagatcctggggcaccagattccatgccaagatcttgctttgcctttgcagctcctccatcacccttctgagcttcttcaactgctccctgtaatccttcggcagcttttgttagttcctttttgaactctttactggagaatatagaagtagctgttttgtctttggtagaatccggagcacctcccttcacaggacgcaatttacccctttgtgcttgcagctcagctgcaaaagagctactagttcctgaactcaggtctttatcagaacctataccttctttagtaggcaaactacttgtcctagctggaacctgaggtttcactttcttcttaatcacagttattgttgagccgactttttcagaagctgttccttctttttgagaagtatcactcttcttaggaccctttttcactgttgcataaatcggctcttccttagggccaaatgtcgttactccagaagatgttcgttccgcagcaaatgggtcagcatagatagattcaggcctttcctgcctaggtttcactatatcaaatggatcagcataaatggattccggcctttctcccttagatgacgccgcatctgatgcttgcgcctcggaagtaattgcagctcccacagtagcatacagatcttcaccttctggtgttctcggaccttcagctcctacagttgtatatgtgctttcaacttccgttgtaccttttgctgtatccttaatttcttcgtagatagactcctcagctcctacagttgtatatgtgctttcaacttccgttgtaccttttgctgtatccttaatttcttcgtagatagactcctcagctcctgcagtatctaggccactacccaaggatgatagcgcagagacactctcaaaacttgaaatagatcctaaagaaggagttggactttcaggcggcagatatggtgggaatcccccttcaggaacttgaacacgttcagccatcattgtgacaacggactttccaaaaaaccacggacgagttttcaatgatggatccgcaacatcgaccggtgtttttccctctacattcacgactgatactgacgccccagactttagtagtattttacatgctttaccgaaaccacgcgatgcagccagatgcagtaacgtgtcaccatttgcttcttgaggagtattaagcaagtctccgaaagatgagtttgacaaatgctttcgagactctttaagcatctttaaaaagcatttttctgtaaccttatcagaatataaagcctcatgtaacgctgtatctcccatatgagaaaggagtgcttgacagctatctgggcattttttcgcaattaacttatatagcttaccgtcaccattagcagctgctatatgtaaagccgtcttaccataagcatctctctgcgttgctggagcccctttatccaagagcaacctagcagtcttctggttgccagcagctgttgctaaatgcaaggctggagttccagtgtgatccgtagacgaaagatctgcacccctctgtaaaaggaaatttacaatcctattagcctctttaaggttacttgcctcatttgccacttgaactgcagcagctaaagggctcatagatccggtaggagtatttatatgtgccccagcttctacaacacgctttaaatgctttatagctttacccccctgaaagcaccctccttgtatacccacagaaatagctggttctggagacgcatttacatcagcactgtttttaattaacgtcttcactgcagcatattgaccactagttagtgcttcagcggtcaaagttgtcttttttccttcaggagttgtaatttcttcatttacactaatcacttcagtggtaataagatgcctcaatacatctgctgcaccttttcttactgcctcgacagcaacatgctgcgggtaaggctcatatctcattaacatgtcaagtgctggtagcgatacttttccaccacttgcttcacgaatcgcatatacacctggagtaggaacaccatcctttacaggaaacttagaataactactcttccttccaagagcctgctgcaatatctctaaatttccatcctttgctgcgtaatgtattatagttccaccatcatgtgaccgagcatctacgtccatgctattacagcgtaacatagtcttaacaccctcagtgttgccccctttatacgcagctaccacaggcgtttcacctgtcactggagatggtacattgattgatggaatattacgcacattctcaatcaacatctgcaatttaacgcttacgcctttatggcttggctcatcctcaactatcatgtgaataggcgctttgccattcggtgctaattgatttacaacagactcaggagtgcatcttaccacctgctcaaaaacccccactgttgatttttgtgctgcagcatgtataggtgcattacctgcaatatctaaattagtaaaaggttcctctccatacctatgatatgcttcctccaatacccttttcgcaagaggatcaaaatttggggtcccattagaagatacaaaatgcaccagcgttgatgcgtcctctggattaggacatgtaaagagagattttacttctgaagaagctgagccatacactttatctgcaatgttcatggccttctcgaagatcttctcagcctccggtatatgccttctaatagcatactgtactgcactcatcccttttttatccgggaatattagtgcctctgcacactcgcgattgccctcaatatttgacgacaccgcttcttgcatcttgtcaatgtatgataaaacatcccgccttggccattgctttgcaacaatgtggcaaacggtttcaccagcatcatttgcaacgctaatatcacttaaccttgagagaagatgctttactttctggtgatccatacgctccgtagcaatatgaagcggagtgtttccacccggtcccttagcattaacatctgctataagagctttgtcgcatagtacatcaagattgcctaaagcatttttgcctactgaagatgcagctgtatgtaatggcgtattaccatcta

SEQ ID NO: 87 - protein, 1054 aa, Ehrlichia (HGE)
DGNTPLHTAASSVGKNALGNLDVLCDKALIADVNAKGPGGNTPLHIATERMDHQKVKHLLSRLSDISVANDAGETVCHIVAKQWPRRDVLSYIDKMQEAVSSNIEGNRECAEALIFPDKKGMSAVQYAIRRHIPEAEKIFEKAMNIADKVYGSASSEVKSLFTCPNPEDASTLVHFVSSNGTPNFDPLAKRVLEEAYHRYGEEPFTNLDIAGNAPIHAAAQKSTVGVFEQVVRCTPESVVNQLAPNGKAPIHMIVEDEPSHKGVSVKLQMLIENVRNIPSINVPSPVTGETPVVAAYKGGNTEGVKTMLRCNSMDVDARSHDGGTIIHYAAKDGNLEILQQALGRKSSYSKFPVKDGVPTPGVYAIREASGGKVSLPALDMLMRYEPYPQHVAVEAVRKGAADVLRHLITTEVISVNEEITTPEGKKTTLTAEALTSGQYAAVKTLIKNSADVNASPEPAISVGIQGGCFQGGKAIKHLKRVVEAGAHINTPTGSMSPLAAAVQVANEASNLKEANRIVNFLLQRGADLSSTDHTGTPALHLATAAGNQKTARLLLDKGAPATQRDAYGKTALHIAAANGDGKLYKLIAKKCPDSCQALLSHMGDTALHEALYSDKVTEKCFLKMLKESRKHLSNSSFGDLLNTPQEANGDTLLHLAASRGFGKACKILLKSGASVSVVNVEGKTPVDVADPSLKTRPWFFGKSVVTMMAERVQVPEGGFPPYLPPESPTPSLGSISSFESVSALSSLGSGLDTAGAEESIYEEIKDTAKGTTEVESTYTTVGAEESIYEEIKDTAKGTTEVESTYTTVGAEGPRTPEGEDLYATVGAAITSEAQASDAASSKGERPESIYADPFDIVKPRQERPESIYADPFAAERTSSGVTTFGPKEEPIYATVKKGPKKSDTSQKEGTASEKVGSTITVIKKKVKPQVPARTSSLPTKEGIGSDKDLSSGTSSSFAAELQAQRGKLRPVKGGAPDSTKDKTATSIFSSKEFKKELTKAAEGLQGAVEEAQKGDGGAAKAKQDLGMESGAPGSQPEAPQSEGPKSVKGGRGR

SEQ ID NO: 88 - DNA, 3735 bp, Ehrlichia
aatgcgctccacataactagcataacgttttcagcaacggcagatcttcatatataagcactgaacacctacgttccaagatcatgctcttcgcgcctgtttacttggtggctcagagtcatcatcactaggagttcgtggtctgtgagagctaacttgtgcttcttccagcgtagaactagcacctcccaatcctgatgctgaaggttgatcccacgaataaggcataatcccttgatcctgaggtggcacatagggagcttgtgatcttcccattccagtactagtacctcctagcccagatgttgagaattggctagatggataaggaacattctctaggacacgtagtagaatatgagggggggggggaacgagttgagctccctgtccggcagtacctcccaatcctgatgttgagggttgatcccatgatgttgagggttgatcccacgatgttgaaggttgtgcatacgaatagggcatcatccctggatcatgtggtggaatatgcgaagcttgttgacttcccattccagcggcacttcctaaccctgatgttgagggttgatcccacgatgttgaaggttgtgcatacgaatagggcatcatccctggatcatgtggtggaatatgcgaagcttgttgacttcccattccagcggcacttcctaaccctgatgttgagggttgatcccacgatgttgaaggttgtgcatacgaatagggcatcatccctggatcatgtggtggaatatgcgaagcttgttgacttcccgttccagcggcacttcctaaccctgatgttgagggttgatcccacaatgttgaaggttgtgcatacgaatagggcatcatccctggatcatgtggtggaatatgcgaagcttgttgacttcccgttccagcagtaccccccattcctgatgttgagggttgatcccacggcgcaccatagggtatgggtatacgctcaagaacacgtagtgggacactgatagcttgtgctccttccactccagcactagtactccctaatcctgatgtcgagggttgactaggtgcagcaccggtctgctcaacagcattgaaatatcttccgtatttcttgtcacaaatattcatcattactgaaagataccgcaatgctgtattgcgccacttgacttctatctgtggaattaatagcgcatcttccgtaatatgctcattgatctcctcatagacatggcacatgtctaaaaatgatttgcgagccctgtatgccccgagctcccttcttctgctatataaagcacacaaaatctggagacaatgcccaatcctacctgcaacaacatgatctacattaccggtggaagcgtatactctatacatcaagaacaaaccacctactgcatgcactaaagcaccaccccgatacctttctcgcttgagtcgtaaatcaaaactgtgaactcctaaaccttcaacatatgcctctaaatagtagagaaaatttgccatcgctcttctagagagtcctagacgcaggcgtgcactttcattattacgtaccatcgcttcacatgcagctgcactagtctcaatagcatcaataacactgtccaagcaagcctctgtacgatgacggaaaaaacgcggtgtattaggctcaactaactcagcaaccttactgcaaagctctatgttatgccgcactacgcgcaaaatcgcctttatattctctgtttcctcagaatccaaagaagaatttaagcatctacttaaggctgaaaattttacatagcagtatgcacttaaagctgtcactgtatgagatgcactaccatctctacgctcactactcactgcaccagtaaacctcgtggcaatagttctggcacagcagttcactatagcaataacattcactatgatagcacatgccttgcctatttgtaggtgtgccttacgcttaataaagtcttgatccatgaacagcggcacttctttgttgcactgcgccgtgatgcagtcctgcaacgcgtcgtacaaccgattgatcaaactatacaacacccccggttctgcgcttgaagcaccttctgcagcagttatacagctgttaatactgtctatcttatcagctgccgcaaacacgacatctacaccccggagcttgacaaacgtatcgcgcaattccagcatacattgacgtatagcctgcaggcatgcagcatatggcctggaattagtcattattgaattacatacagtttctttatattccgcagaagagcaaccactgtaggcatatccagacataactggagtagtgaatatacgaggcatatgcatctaattaaccactggaacaacttcacaccttgaaagtgtagcataccggtgtgacgcagctcaatattaaagattatgcacttcgtgatcgtctactaggaggctcaagttcatcatcactaggagtttgtgatctaggagagactacctgtgctccttccagcgtagaactagcacctcctaatcctgatgttgagggttgtgcatacgaataatcttgcaacggaccacaaggtgcctgagcttgcagtgctccctgtccagcaggattacctcccaatcccgatgttgagggttgactaggtgaagagggcatatgccctggatcatgaggtagcgtataggaagcttgtgatcctcctattccagccccagcacttcctagtctagatgttgagggttgactaggcgaaccctcagtctgcctaatattattgaaatatctctcgtacttcttttcccaaataccaatcattgccgaaagataccccaacatagcactacagaacccaacttctgtctggggatttaatagtagacctcgcgtaacgcattcctgaatctcatcatagacagtacacatgtccaaatataattcttgtgccgtatattctgaagctcccgctcttctgaccttatatttatagagagtaagcaacatttgaagacaatgctcaattttactcgcaacaacatgccctgtattacccgtggaagcatatactctgtgcattgagaataaactaccaattgcatacactaaagcttgcacatacttgtcatgcctgaaacttttaaaagcaacgctcagtcctaaacttttatatgtcttgaaatggtgtaaaaaacctgttctcgcttttttagcgagagctaggcggttctttgcactatcgttatcactcaccatctcttcgcattcagccgaggtagacccaactgcatcaagcatactgtttaagcaactcaccgtacgatcacggaaacaatatggaatctccggatcaactagctcagcaaccttattacaaagctctatgttatgcctcaccacacgtagaatagcctttctacgcttagtttcctcaggacccggagaataatttaaacatctgcttaaagctgaaaattttgcatttacgtatgcacttaaagccatgttggcatgatacgcactatgctcatcagcctcacctattgcactgtcagacgcctcggttaaggttgtgacaaagcagcttgccatggtaatagcattcaccaggatagcacataccttagcgatttgtaggtgtacttcacgcctcgtgaagtctggatccatgaaccgcggcacttctttgttgcactgcgccgtggcacagtcatgcagcatattatatgcactatggatta

SEQ ID NO: 89 - protein, 752 aa, Ehrlichia
MHMPRIFTTPVMSGYAYSGCSSAEYKETVCNSIMTNSRPYAACLQAIRQCMLELRDTFVKLRGVDVVFAAADKIDSINSCITAAEGASSAEPGVLYSLINRLYDALQDCITAQCNKEVPLFMDQDFIKRKAHLQIGKACAIIVNVIAIVNCCARTIATRFTGAVSSERRDGSASHTVTALSAYCYVKFSALSRCLNSSLDSEETENIKAILRVVRHNIELCSKVAELVEPNTPRFFRHRTEACLDSVIDAIETSAAACEAMVRNNESARLRLGLSRRAMANFLYYLEAYVEGLGVHSFDLRLKRERYRGGALVHAVGGLFLMYRVYASTGNVDHVVAGRIGHCLQILCALYSRRRELGAYRARKSFLDMCHVYEEINEHITEDALLIPQIEVKWRNTALRYLSVMMNICDKKYGRYFNAVEQTGAAPSQPSTSGLGSTSAGVEGAQAISVPLRVLERIPIPYGAPWDQPSTSGMGGTAGTGSQQASHIPPHDPGMMPYSYAQPSTLWDQPSTSGLGSAAGTGSQQASHIPPHDPGMMPYSYAQPSTSWDQPSTSGLGSAAGMGSQQASHIPPHDPGMMPYSYAQPSTSWDQPSTSGLGSAAGMGSQQASHIPPHDPGMMPYSYAQPSTSWDQPSTSWDQPSTSGLGGTAGQGAQLVPPPPHILLRVLENVPYPSSQFSTSGLGGTSTGMGRSQAPYVPPQDQGIMPYSWDQPSASGLGGASSTLEEAQVSSHRPRTPSDDDSEPPSKQARRA

SEQ ID NO: 90 - DNA, 2142 bp, Ehrlichia
atgcagcatcaccaccatcaccacaaaggggctccagcaacgcagagagatgcttatggtaagacggctttacatatagcagctgctaatggtgacggtaagctatataagttaattgcgaaaaaatgcccagatagctgtcaagcactcctttctcatatgggagatacagcgttacatgaggctttatattctgataaggttacagaaaaatgctttttaaagatgcttaaagagtctcgaaagcatttgtcaaactcatctttcggagacttgcttaatactcctcaagaagcaaatggtgacacgttactgcatctggctgcatcgcgtggtttcggtaaagcatgtaaaatactactaaagtctggggcgtcagtatcagtcgtgaatgtagagggaaaaacaccggtagatgttgcggatccatcattgaaaactcgtccgtggttttttggaaagtccgttgtcacaatgatggctgaacgtgttcaagttcctgaagggggattcccaccatatctgccgcctgaaagtccaactccttctttaggatctatttcaagttttgagagtgtctctgcgctatcatccttgggtagtggcctagatactgcaggagctgaggagtctatctacgaagaaattaaggatacagcaaaaggtacaacggaagttgaaagcacatatacaactgtaggagctgaggagtctatctacgaagaaattaaggatacagcaaaaggtacaacggaagttgaaagcacatatacaactgtaggagctgaaggtccgagaacaccagaaggtgaagatctgtatgctactgtgggagctgcaattacttccgaggcgcaagcatcagatgcggcgtcatctaagggagaaaggccggaatccatttatgctgatccatttgatatagtgaaacctaggcaggaaaggcctgaatctatctatgctgacccatttgctgcggaacgaacatcttctggagtaacgacatttggccctaaggaagagccgatttatgcaacagtgaaaaagggtcctaagaagagtgatacttctcaaaaagaaggaacagcttctgaaaaagtcggctcaacaataactgtgattaagaagaaagtgaaacctcaggttccagctactcgatcggagcttgagattggttacgagcgcttcaagaccaagggtattagagatagtggtagtaaggaagatgaagctgatacagtatatctactagctaaggagttagcttatgatgttgttactggtcagactgataaccttgccgctgctcttgccaaaacctccggtaaggatattgttcagtttgctaaggcggtggagatttctcattccgagattgatggcaaggtttgtaagacgaagtcggcgggaactggaaaaaatccgtgtgatcatagccaaaagccgtgtagtacgaatgcgtattatgcgaggagaacgcagaagagtaggagttcgggaaaaacgtctttatgcggggacagtgggtatagcgggcaggagctaataacgggtgggcattatagcagtccaagcgtattccggaattttgtcaaagacacactacaaggaaatggtagtgagaactggcctacatctactggagaaggaagtgagagtaacgacaacgccatagccgttgctaaggacctagtaaatgaacttactcctgaagaacgaaccatagtggctgggttacttgctaaaattattgaaggaagcgaggttattgagattagggccatctcttcgacttcagttacaatgaatatttgctcagatatcacgataagtaatatcttaatgccgtatgtttgtgttggtccagggatgagctttgttagtgttgttgatggtcacactgctgcaaagtttgcatatcggttaaaggcaggtctgagttataaattttcgaaagaagttacagcttttgcaggtggtttttaccatcacgttataggagatggtgtttatgatgatctgccattgcggcatttatctgatgatattagtcctgtgaaacatgctaaggaaaccgccattgctagattcgtcatgaggtactttggcggggaatttggtgttaggctcgctttttaatga

SEQ ID NO: 91 - DNA, 2133 bp, Ehrlichia
atgcagcatcaccaccatcaccacaaaggggctccagcaacgcagagagatgcttatggtaagacggctttacatatagcagctgctaatggtgacggtaagctatataagttaattgcgaaaaaatgcccagatagctgtcaagcactcctttctcatatgggagatacagcgttacatgaggctttatattctgataaggttacagaaaaatgctttttaaagatgcttaaagagtctcgaaagcatttgtcaaactcatctttcggagacttgcttaatactcctcaagaagcaaatggtgacacgttactgcatctggctgcatcgcgtggtttcggtaaagcatgtaaaatactactaaagtctggggcgtcagtatcagtcgtgaatgtagagggaaaaacaccggtagatgttgcggatccatcattgaaaactcgtccgtggttttttggaaagtccgttgtcacaatgatggctgaacgtgttcaagttcctgaagggggattcccaccatatctgccgcctgaaagtccaactccttctttaggatctatttcaagttttgagagtgtctctgcgctatcatccttgggtagtggcctagatactgcaggagctgaggagtctatctacgaagaaattaaggatacagcaaaaggtacaacggaagttgaaagcacatatacaactgtaggagctgaggagtctatctacgaagaaattaaggatacagcaaaaggtacaacggaagttgaaagcacatatacaactgtaggagctgaaggtccgagaacaccagaaggtgaagatctgtatgctactgtgggagctgcaattacttccgaggcgcaagcatcagatgcggcgtcatctaagggagaaaggccggaatccatttatgctgatccatttgatatagtgaaacctaggcaggaaaggcctgaatctatctatgctgacccatttgctgcggaacgaacatcttctggagtaacgacatttggccctaaggaagagccgatttatgcaacagtgaaaaagggtcctaagaagagtgatacttctcaaaaagaaggaacagcttctgaaaaagtcggctcaacaataactgtgattaagaagaaagtgaaacctcaggttccagctactcgatcgttctatattggtttggattacagtccagcgtttagcaagataagagattttagtataagggagagtaacggagagacaaaggcagtatatccatacttaaaggatggaaagagtgtaaagctagagtcacacaagtttgactggaacacacctgatcctcggattgggtttaaggacaacatgcttgtagctatggaaggtagtgttggttatggtattggtggtgccagggttgagcttgagattggttacgagcgcttcaagaccaagggtattagagatagtggtagtaaggaagatgaagctgatacagtatatctactagctaaggagttagcttatgatgttgttactggacagactgataaccttgctgctgctcttgctaagacctcggggaaagacatcgttcagtttgctaaggcggttggggtttctcatcctagtattgatgggaaggtttgtaagacgaaggcggatagctcgaagaaatttccgttatatagtgacgaaacgcacacgaagggggcaaatgaggggagaacgtctttgtgcggtgacaatggtagttctacgataacaaccagtggtacgaatgtaagtgaaactgggcaggtttttagggattttatcagggcaacgctgaaagaggatggtagtaaaaactggccaacttcaagcggcacgggaactccaaaacctgtcacgaacgacaacgccaaagccgtagctaaagacctagtacaggagctaacccctgaagaaaaaaccatagtagcagggttactagctaagactattgaagggggtgaagttgttgagatcagggcggtttcttctacttccgtaatggtcaatgcttgttatgatcttcttagtgaaggtttaggtgttgttccttatgcttgtgttggtctcggtggtaacttcgtgggcgtggttgatggaattcattacacaaaccatctttaa

SEQ ID NO: 92 - protein, 712 aa, Ehrlichia
MQHHHHHHKGAPATQRDAYGKTALHIAAANGDGKLYKLIAKKCPDSCQALLSHMGDTALHEALYSDKVTEKCFLKMLKESRKHLSNSSFGDLLNTPQEANGDTLLHLAASRGFGKACKILLKSGASVSVVNVEGKTPVDVADPSLKTRPWFFGKSVVTMMAERVQVPEGGFPPYLPPESPTPSLGSISSFESVSALSSLGSGLDTAGAEESIYEEIKDTAKGTTEVESTYTTVGAEESIYEEIKDTAKGTTEVESTYTTVGAEGPRTPEGEDLYATVGAAITSEAQASDAASSKGERPESIYADPFDIVKPRQERPESIYADPFAAERTSSGVTTFGPKEEPIYATVKKGPKKSDTSQKEGTASEKVGSTITVIKKKVKPQVPATRSELEIGYERFKTKGIRDSGSKEDEADTVYLLAKELAYDVVTGQTDNLAAALAKTSGKDIVQFAKAVEISHSEIDGKVCKTKSAGTGKNPCDHSQKPCSTNAYYARRTQKSRSSGKTSLCGDSGYSGQELITGGHYSSPSVFRNFVKDTLQGNGSENWPTSTGEGSESNDNAIAVAKDLVNELTPEERTIVAGLLAKIIEGSEVIEIRAISSTSVTMNICSDITISNILMPYVCVGPGMSFVSVVDGHTAAKFAYRLKAGLSYKFSKEVTAFAGGFYHHVIGDGVYDDLPLRHLSDDISPVKHAKETAIARFVMRYFGGEFGVRLAF

SEQ ID NO: 93 - protein, 658 aa, Ehrlichia; identical to SEQ ID NO: 85

SEQ ID NO: 94 - DNA, 1080 bp, Ehrlichia
ttgagcttgagattggttacgagcgcttcaagaccaagggtattagagatagtggtagtaaggaagatgaagctgatacagtatatctactagctaaggagttagcttatgatgttgttactggtcagactgataaccttgccgctgctcttgccaaaacctccggtaaggatattgttcagtttgctaaggcggtggagatttctcattccgagattgatggcaaggtttgtaagacgaagtcggcgggaactggaaaaaatccgtgtgatcatagccaaaagccgtgtagtacgaatgcgtattatgcgaggagaacgcagaagagtaggagttcgggaaaaacgtctttatgcggggacagtgggtatagcgggcaggagctaataacgggtgggcattatagcagtccaagcgtattccggaattttgtcaaagacacactacaaggaaatggtagtgagaactggcctacatctactggagaaggaagtgagagtaacgacaacgccatagccgttgctaaggacctagtaaatgaacttactcctgaagaacgaaccatagtggctgggttacttgctaaaattattgaaggaagcgaggttattgagattagggccatctcttcgacttcagttacaatgaatatttgctcagatatcacgataagtaatatcttaatgccgtatgtttgtgttggtccagggatgagctttgttagtgttgttgatggtcacactgctgcaaagtttgcatatcggttaaaggcaggtctgagttataaattttcgaaagaagttacagcttttgcaggtggtttttaccatcacgttataggagatggtgtttatgatgatctgccattgcggcatttatctgatgatattagtcctgtgaaacatgctaaggaaaccgccattgctagattcgtcatgaggtactttggcggggaatttggtgttaggctcgctttttaaggttgcgacctaaaagcacttagctcgccttcactcccccttaagcaatatgatgcacatttgttgccctacaaatctaatataaggtttgttgcctata

SEQ ID NO: 95 - DNA, 2120 bp, Ehrlichia
gaaacagcattgctagatttcgttgaacaatttgctaatttgcaactaaagcactcatgataaagcttgatagtattttagaggatagtaggcaatatggtttaggggatttcttcgcatacttgttatcatcgtccttatttgtgcttagttggtcggatatttgtgcaagttgttgtaaaatatgcatattgtatgtataggtgtgcaagatatcatctctttaggtgtatcgtgtagcacttaaacaaatgctggtgaacgtagagggattaaaggaggatttgcgtatatgtatggtatagatatagagctaagtgattacagaattggtagtgaaaccatttccagtggagatgatggctactacgaaggatgtgcttgtgacaaagatgccagcactaatgcgtactcgtatgacaagtgtagggtagtacggggaacgtggagaccgagcgaactggttttatatgttggtgatgagcatgtggcatgtagagatgttgcttcgggtatgcatcatggtaatttgccagggaaggtgtattttatagaggcagaagcgggcagagctgctactgctgaaggtggtgtttatactaccgttgtggaggcattatcgctggtgcaagaggaagagggtacaggtatgtacttgataaacgcaccagaaaaagcggtcgtaaggtttttcaagatagaaaagagtgcagcagaggaacctcaaacagtagatcctagtgtagttgagtcagcaacagggtcgggtgtagatacgcaagaagaacaagaaatagatcaagaagcaccagcaattgaagaagttgagacagaagagcaagaagttattctggaagaaggtactttgatagatcttgagcaacctgtagcgcaagtacctgtagtagctgaagcagaattacctggtgttgaagctgcagaagcgattgtaccatcactagaagaaaataagcttcaagaagtggtagttgctccagaagcgcaacaactagaatcagctcctgaagtttctgcgccagcacaacctgagtctacagttcttggtgttgctgaaggtgatctaaagtctgaagtatctgtagaagctaatgctgatgtagcgcaaaaagaagtaatctctggtcaacaagagcaagaaattgcagaagcactagagggaactgaagctcctgtagaagtaaaagaagaaacagaagttcttctaaaggaagatactttgatagatcttgagcaacctgtagcacaagtacctgtagtagctgaagcagaattacctggtgttgaagctgcagaagcgattgtaccatcactagaagaaaataagcttcaagaagtggtagttgctccagaagcgcaacaactagaatcagctcctgaagtttctgcaccagcacaacctgagtctacagttcttggtgttactgaaggtgatctgaagtctgaagtatctgtagaagctgatgctggtatgcagcaagaagcaggaatctctgatcaagagacacaagcaactgaagaagttgaaaaggttgaagtatctgtagaaacaaaaacggaagagccagaagttattctagaagaaggtactttgatagatcttgagcaacctgtagcgcaagtacctgtagtagctgaagcagaattacctggtgttgaagctgcagaagcgattgtaccatcactagaagaaaataagcttcaagaagtggtagttgctccagaagcgcaacaactagaatcagctcctgaagtttctgcgccagtacaacctgagtctacagttcttggtgttactgaaggtgatctgaagtctgaagtatctgtagaagctgatgctggtatgcagcaagaagcaggaatctctgatcaagagacacaagcaactgaagaagttgagaaggttgaagtatctgtagaagctgatgctggtatgcagcaagagttagtagatgttccgactgctttgccgttaaaggatcctgacgatgaagatgttctaagttattaggatatctttctcgtgaaaagtatggggaaggttcgatgtgttggaccgtgccccatgctttttctttaagatttcttcaaaaagaggtaaa

SEQ ID NO: 96 - DNA, 3735 bp, Ehrlichia
taatccatagtgcatataatatgctgcatgactgtgccacggcgcagtgcaacaaagaagtgccgcggttcatggatccagacttcacgaggcgtgaagtacacctacaaatcgctaaggtatgtgctatcctggtgaatgctattaccatggcaagctgctttgtcacaaccttaaccgaggcgtctgacagtgcaataggtgaggctgatgagcatagtgcgtatcatgccaacatggctttaagtgcatacgtaaatgcaaaattttcagctttaagcagatgtttaaattattctccgggtcctgaggaaactaagcgtagaaaggctattctacgtgtggtgaggcataacatagagctttgtaataaggttgctgagctagttgatccggagattccatattgtttccgtgatcgtacggtgagttgcttaaacagtatgcttgatgcagttgggtctacctcggctgaatgcgaagagatggtgagtgataacgatagtgcaaagaaccgcctagctctcgctaaaaaagcgagaacaggttttttacaccatttcaagacatataaaagtttaggactgagcgttgcttttaaaagtttcaggcatgacaagtatgtgcaagctttagtgtatgcaattggtagtttattctcaatgcacagagtatatgcttccacgggtaatacagggcatgttgttgcgagtaaaattgagcattgtcttcaaatgttgcttactctctataaatataaggtcagaagagcgggagcttcagaatatacggcacaagaattatatttggacatgtgtactgtctatgatgagattcaggaatgcgttacgcgaggtctactattaaatccccagacagaagttgggttctgtagtgctatgttggggtatctttcggcaatgattggtatttgggaaaagaagtacgagagatatttcaataatattaggcagactgagggttcgcctagtcaaccctcaacatctagactaggaagtgctggggctggaataggaggatcacaagcttcctatacgctacctcatgatccagggcatatgccctcttcacctagtcaaccctcaacatcgggattgggaggtaatcctgctggacagggagcactgcaagctcaggcaccttgtggtccgttgcaagattattcgtatgcacaaccctcaacatcaggattaggaggtgctagttctacgctggaaggagcacaggtagtctctcctagatcacaaactcctagtgatgatgaacttgagcctcctagtagacgatcacgaagtgcataatctttaatattgagctgcgtcacaccggtatgctacactttcaaggtgtgaagttgttccagtggttaattagatgcatatgcctcgtatattcactactccagttatgtctggatatgcctacagtggttgctcttctgcggaatataaagaaactgtatgtaattcaataatgactaattccaggccatatgctgcatgcctgcaggctatacgtcaatgtatgctggaattgcgcgatacgtttgtcaagctccggggtgtagatgtcgtgtttgcggcagctgataagatagacagtattaacagctgtataactgctgcagaaggtgcttcaagcgcagaaccgggggtgttgtatagtttgatcaatcggttgtacgacgcgttgcaggactgcatcacggcgcagtgcaacaaagaagtgccgctgttcatggatcaagactttattaagcgtaaggcacacctacaaataggcaaggcatgtgctatcatagtgaatgttattgctatagtgaactgctgtgccagaactattgccacgaggtttactggtgcagtgagtagtgagcgtagagatggtagtgcatctcatacagtgacagctttaagtgcatactgctatgtaaaattttcagccttaagtagatgcttaaattcttctttggattctgaggaaacagagaatataaaggcgattttgcgcgtagtgcggcataacatagagctttgcagtaaggttgctgagttagttgagcctaatacaccgcgttttttccgtcatcgtacagaggcttgcttggacagtgttattgatgctattgagactagtgcagctgcatgtgaagcgatggtacgtaataatgaaagtgcacgcctgcgtctaggactctctagaagagcgatggcaaattttctctactatttagaggcatatgttgaaggtttaggagttcacagttttgatttacgactcaagcgagaaaggtatcggggtggtgctttagtgcatgcagtaggtggtttgttcttgatgtatagagtatacgcttccaccggtaatgtagatcatgttgttgcaggtaggattgggcattgtctccagattttgtgtgctttatatagcagaagaagggagctcggggcatacagggctcgcaaatcatttttagacatgtgccatgtctatgaggagatcaatgagcatattacggaagatgcgctattaattccacagatagaagtcaagtggcgcaatacagcattgcggtatctttcagtaatgatgaatatttgtgacaagaaatacggaagatatttcaatgctgttgagcagaccggtgctgcacctagtcaaccctcgacatcaggattagggagtactagtgctggagtggaaggagcacaagctatcagtgtcccactacgtgttcttgagcgtatacccataccctatggtgcgccgtgggatcaaccctcaacatcaggaatggggggtactgctggaacgggaagtcaacaagcttcgcatattccaccacatgatccagggatgatgccctattcgtatgcacaaccttcaacattgtgggatcaaccctcaacatcagggttaggaagtgccgctggaacgggaagtcaacaagcttcgcatattccaccacatgatccagggatgatgccctattcgtatgcacaaccttcaacatcgtgggatcaaccctcaacatcagggttaggaagtgccgctggaatgggaagtcaacaagcttcgcatattccaccacatgatccagggatgatgccctattcgtatgcacaaccttcaacatcgtgggatcaaccctcaacatcagggttaggaagtgccgctggaatgggaagtcaacaagcttcgcatattccaccacatgatccagggatgatgccctattcgtatgcacaaccttcaacatcgtgggatcaaccctcaacatcatgggatcaaccctcaacatcaggattgggaggtactgccggacagggagctcaactcgttccccccccccctcatattctactacgtgtcctagagaatgttccttatccatctagccaattctcaacatctgggctaggaggtactagtactggaatgggaagatcacaagctccctatgtgccacctcaggatcaagggattatgccttattcgtgggatcaaccttcagcatcaggattgggaggtgctagttctacgctggaagaagcacaagttagctctcacagaccacgaactcctagtgatgatgactctgagccaccaagtaaacaggcgcgaagagcatgatcttggaacgtaggtgttcagtgcttatatatgaagatctgccgttgctgaaaacgttatgctagttatgtggagcgcatt

SEQ ID NO: 97 - DNA, 2008 bp, Ehrlichia
atgcttatgtagaattctgcacaagcagcagaatggtgctttcattaacacggatgtatatgggatgggtaagggctcttaagctttgcatggcaaggttctatagctttttagaacttcatatatcgtaccgaaacaaattaatacgggtctatccatacattacgtaatggctactatgcaaaattcagaatattgcccataaacaactagaaaaagtcttgcagattttttctgattactatattccttcgggaatctgaccagctatgggcgttctgttatgcgatcaaggaagatttatgtttgggtggtcatggcaacggttttaggtgccatggcttttgtcacttttggaagcatgataccaatgggtaagttgtctaattctggcaacggacagtgcgttgcaatgttgggtaataaatgtctaccattgcgggattaccgtataatgtaccgcaacgagttggcagaactagagaagatgttacaacacaaattgtctgatgctcaaattaatcagtttggtattaaggaagttgtcctcaagaacatgatagccgacatggtcgttgaaaagtttgctcatgacttaggcatacgtgttggctcaaatagcttacggagtctgatcaaaaatataagaatatttcaggatgctaatggtgtcttcgaccaggagagatatgaagccgtattggctgacagcggaatgactgagtcgtcctatgtgaataaaattcgcaatgctttaccttctactattctaatggagtgtttattccctaatagggcggaattacatattccttattatgatgcattagcaaaagatgttgtgttgggattgctgcagcatcgtgtggcagacatagtggaaatatcttctgatgccgtagacatttcaggaagtgatatatctgatgatgaattgcaaaaattgtttgaggagcagtacaagaattctctaaatttccctgaatatcgcagtgctgattatataatcatggcagaagacgacttgcttgctgatgtcattgtttcggatcaagaggtagacgttgagattaaaaacagtgaactacatgatcaaagagatgttctaaatttagtatttacagacaaaaatgaagctgagctagcttacaaagcttaccaagagggtaagtcttttgaggaattggttagtgatgctggctacaccatagaggatattgcactcaataatatctctaaggatgttcttccggtaggtgtgcgaaatgtggtgtttgcactaaatgaaggagaagtcagtgaaatgttccgtagcgttgtcggctggcatatcatgaaggtaataaggaagcatgagatcactaaggaagacctagaaaagctgaaagagaagatatcttcaaatattagaaggcaaaaggcaggtgagttgctagttagcaatgtgaaaaaagcaaacgatatgatcagccgcggggcattgctgaatgaactaaaggatatgtttggtgcgcggatcagtggtgttttgacgaattttgatatgcatgggctcgataaatctggcaacttagtgaaagactttccgttgcagcttggtataaacgcctttactactttggcgttttcatctgccgtaggaaaaccgtctcatctggttagcaatggtgacgcttatttcggcgttcttgttactgaagtagtgcctccaagaccaaggacacttgaagaaagcaggtctattcttactgaagaatggaagagtgcattacgtatgaagaaaatacgtgaatttgctgtggagttgcgctcgaagctacaaaatggcactgaattgtccgttgtaaatggagtttcttttaaaaagaatgtcacggtaaaaaagtcagatggctctaccgacaatgatagcaagtatcctgaacgcttagtcgatgagatattcgccattaacattggtggagtaacgaaagaagttatagattctgaatctgagactgtatacatt

SEQ ID NO: 98 - DNA, 3300 bp, Ehrlichia
tagatggtaatacgccattacatacagctgcatcttcagtaggcaaaaatgctttaggcaatcttgatgtactatgcgacaaagctcttatagcagatgttaatgctaagggaccgggtggaaacactccgcttcatattgctacggagcgtatggatcaccagaaagtaaagcatcttctctcaaggttaagtgatattagcgttgcaaatgatgctggtgaaaccgtttgccacattgttgcaaagcaatggccaaggcgggatgttttatcatacattgacaagatgcaagaagcggtgtcgtcaaatattgagggcaatcgcgagtgtgcagaggcactaatattcccggataaaaaagggatgagtgcagtacagtatgctattagaaggcatataccggaggctgagaagatcttcgagaaggccatgaacattgcagataaagtgtatggctcagcttcttcagaagtaaaatctctctttacatgtcctaatccagaggacgcatcaacgctggtgcattttgtatcttctaatgggaccccaaattttgatcctcttgcgaaaagggtattggaggaagcatatcataggtatggagaggaaccttttactaatttagatattgcaggtaatgcacctatacatgctgcagcacaaaaatcaacagtgggggtttttgagcaggtggtaagatgcactcctgagtctgttgtaaatcaattagcaccgaatggcaaagcgcctattcacatgatagttgaggatgagccaagccataaaggcgtaagcgttaaattgcagatgttgattgagaatgtgcgtaatattccatcaatcaatgtaccatctccagtgacaggtgaaacgcctgtggtagctgcgtataaagggggcaacactgagggtgttaagactatgttacgctgtaatagcatggacgtagatgctcggtcacatgatggtggaactataatacattacgcagcaaaggatggaaatttagagatattgcagcaggctcttggaaggaagagtagttattctaagtttcctgtaaaggatggtgttcctactccaggtgtatatgcgattcgtgaagcaagtggtggaaaagtatcgctaccagcacttgacatgttaatgagatatgagccttacccgcagcatgttgctgtcgaggcagtaagaaaaggtgcagcagatgtattgaggcatcttattaccactgaagtgattagtgtaaatgaagaaattacaactcctgaaggaaaaaagacaactttgaccgctgaagcactaactagtggtcaatatgctgcagtgaagacgttaattaaaaacagtgctgatgtaaatgcgtctccagaaccagctatttctgtgggtatacaaggagggtgctttcaggggggtaaagctataaagcatttaaagcgtgttgtagaagctggggcacatataaatactcctaccggatctatgagccctttagctgctgcagttcaagtggcaaatgaggcaagtaaccttaaagaggctaataggattgtaaatttccttttacagaggggtgcagatctttcgtctacggatcacactggaactccagccttgcatttagcaacagctgctggcaaccagaagactgctaggttgctcttggataaaggggctccagcaacgcagagagatgcttatggtaagacggctttacatatagcagctgctaatggtgacggtaagctatataagttaattgcgaaaaaatgcccagatagctgtcaagcactcctttctcatatgggagatacagcgttacatgaggctttatattctgataaggttacagaaaaatgctttttaaagatgcttaaagagtctcgaaagcatttgtcaaactcatctttcggagacttgcttaatactcctcaagaagcaaatggtgacacgttactgcatctggctgcatcgcgtggtttcggtaaagcatgtaaaatactactaaagtctggggcgtcagtatcagtcgtgaatgtagagggaaaaacaccggtcgatgttgcggatccatcattgaaaactcgtccgtggttttttggaaagtccgttgtcacaatgatggctgaacgtgttcaagttcctgaagggggattcccaccatatctgccgcctgaaagtccaactccttctttaggatctatttcaagttttgagagtgtctctgcgctatcatccttgggtagtggcctagatactgcaggagctgaggagtctatctacgaagaaattaaggatacagcaaaaggtacaacggaagttgaaagcacatatacaactgtaggagctgaggagtctatctacgaagaaattaaggatacagcaaaaggtacaacggaagttgaaagcacatatacaactgtaggagctgaaggtccgagaacaccagaaggtgaagatctgtatgctactgtgggagctgcaattacttccgaggcgcaagcatcagatgcggcgtcatctaagggagaaaggccggaatccatttatgctgatccatttgatatagtgaaacctaggcaggaaaggcctgaatctatctatgctgacccatttgctgcggaacgaacatcttctggagtaacgacatttggccctaaggaagagccgatttatgcaacagtgaaaaagggtcctaagaagagtgatacttctcaaaaagaaggaacagcttctgaaaaagtcggctcaacaataactgtgattaagaagaaagtgaaacctcaggttccagctaggacaagtagtttgcctactaaagaaggtataggttctgataaagacctgagttcaggaactagtagctcttttgcagctgagctgcaagcacaaaggggtaaattgcgtcctgtgaagggaggtgctccggattctaccaaagacaaaacagctacttctatattctccagtaaagagttcaaaaaggaactaacaaaagctgccgaaggattacagggagcagttgaagaagctcagaagggtgatggaggagctgcaaaggcaaagcaagatcttggcatggaatctggtgccccaggatctcaaccagaagctcctcaaagtgaaggccctaagtctgtaaaaggaggtcgcggtaggtagaattataccgaaaaatcgctgaggtactttgatcaatataattcgcgcttctgagtatttaggcgatgatctcgccactttaataataccccttttagagtacataacgctctaaagggggcagattatttta

SEQ ID NO: 99 - protein, 168 aa, Ehrlichia sp.
LLAKELAYDVVTGQTDNLAAALAKTSGKDIVQFAKAVEISHSEIDGKVCKTKSAGTGKNPCDHSQKPCSTNAYYARRTQKSRSSGKTSLCGDSGYSGQELITGGHYSSPSVFRNFVKDTLQGNGSENWPTSTGEGSESNDNAIAVAKDLVNELTPEERTIVAGLLAKI

SEQ ID NO: 100 - protein, 160 aa, Ehrlichia sp.
LLAKELAYDVVTGQTDNLAAALAKTSGKDIVQFAKAVGVSHPSIDGKVCKTKADSSKKFPLYSDETHTKGANEGRTSLCGDNGSSTITTSGTNVSETGQVFRDFIRATLKEDGSKNWPTSSGTGTPKPVTNDNAKAVAKDLVQELTPEEKTIVAGLLAKT

SEQ ID NO: 101 - protein, 147 aa, Ehrlichia sp.
LLAKELAYDVVTGQTDNLAAALAKTSGKDIVQFAKTLNISHSNIDGKVCRREKHGSQGLTGTKAGSCDSQPQTAGFDSMKQGLMAALGEQGAEKWPKINNGGHATIYSSSAGPGNAYARDASTTVATDLTKLTTEEKTIVAGLLART

SEQ ID NO: 102 - protein, 123 aa, Ehrlichia sp.
AVKITNSTIDGKVCNGSREKGNSAGNNNSAVATYAQTHTANTSTSQCSGLGTTVVKQGYGSLNKFVSLTGVGEGKNWPTGKIHDGSSGVKDGEQNGNAKAVAKDLVDLNRDEKTIVAGLLAKT

SEQ ID NO: 103 - protein, 147 aa, Ehrlichia sp.
LLAKELAYDVVTGQTDNLAAALAKTSGKDIVQFANAVKITNSAIDGKICNRGKASGGSKGLSSSKAGSCDSIDKQSGSLEQSLTAALGDKGAEKWPKINNGTSDTTLNGNDTSSTPYTKDASATVAKDLVALNHDEKTIVAGLLAKT

SEQ ID NO: 104 - protein, 45 aa, Ehrlichia sp.
LLAKELAYDVVTGQTDNLAAALAKTSGKDFVQFAKAVEISNSTIG

SEQ ID NO: 105 - protein, 150 aa, Ehrlichia sp.
LLAKELAYDVVTGQTDNLAAALAKTSGKDFVKFANAVVGISHPDVNKKVCATRKDSGGTRYAKYAATTNKSSNPETSLCGDEGGSSGTNNTQEFLKEFVAKTLVENESKNWPTSSGTGLKTNDNAKAVATDLVALNRDEKTIVAGLLAKT

SEQ ID NO: 106 - protein, 161 aa, Ehrlichia sp.
LLAKELAYDVVTGQTDKLTAALAKTSGKDIVQFAKAVGVSHPSIDGKVCRTKRKAGDSSGTYAKYGEETDNNTSGQSTVAVCGEKAGHNANGSGTVQSLKDFVRETLKADGNRNWPTSREKSGNTNTKPQPNDNAKAVAKDLVQELNHDEKTIVAGLLAKT

SEQ ID NO: 107 - protein, 43 aa, Ehrlichia sp.
LLAKELAYDVVTGQTDNLAAALAKTSGKDFVQFANAVKISAPN

SEQ ID NO: 108 - protein, 156 aa, Ehrlichia sp.
LLAKELAYDVVTGQTDKLTAALAKTSGKDFVQFAKAVGVSHPNIDGKVCKTTLGHTSADSYGVYGELTGQASASETSLCGGKGKNSSGGGAAPEVLRDFVKKSLKDGGQNWPTSRATESSPKTKSETNDNAKAVAKDLVDLNPEEKTIVAGLLAKT

(19) United States
(12) Patent Application Publication
(10) Pub. No.: US 2002/0068343 A1
(43) Pub. Date: Jun. 6, 2002

(54) Compounds and methods for the diagnosis and treatment of ehrlichia infection

(76) Inventors: Steven G. Reed, Bellevue, WA (US); Michael J. Lodes, Seattle, WA (US); Raymond L. Houghton, Bothell, WA (US); Patricia D. McNeill, Des Moines, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/798,042

(22) Filed: Mar. 2, 2001

Related U.S. Application Data
(63) Continuation-in-part of application No. 09/693,542, filed on Oct. 20, 2000.
Application No. 09/693,542 is a continuation-in-part of application No. 09/566,617, filed on May 8, 2000.
Application No. 09/566,617 is a continuation-in-part of application No. 09/295,028, filed on Apr. 20, 1999, now Pat. No. 6,277,381.
Application No. 09/295,028 is a continuation-in-part of application No. 09/159,469, filed on Sep. 23, 1998.
Application No. 09/159,469 is a continuation-in-part of application No. 09/106,582, filed on Jun. 29, 1998, now Pat. No. 6,306,402.
Application No. 09/106,582 is a continuation-in-part of application No. 08/975,762, filed on Nov. 20, 1997, now Pat. No. 6,207,169.
Application No. 08/975,762 is a continuation-in-part of application No. 08/821,324, filed on Mar. 21, 1997, now Pat. No. 6,231,869.

(51) Int. Cl. C12N 9/00; C07H 21/04; C12P 21/02; C12N 5/06

(52) U.S. Cl. 435/183; 536/23.1; 435/69.1; 435/325

(57) ABSTRACT

Compounds and methods for the diagnosis and treatment of Ehrlichia infection, in particular human granulocytic ehrlichiosis, are disclosed. The compounds provided include polypeptides that contain at least one antigenic portion of an Ehrlichia antigen and DNA sequences encoding such polypeptides. Pharmaceutical compositions and vaccines comprising such polypeptides or DNA sequences are also provided. Diagnostic kits containing such polypeptides or DNA sequences and a suitable detection reagent may be used for the detection of Ehrlichia infection in patients and biological samples. Antibodies directed against such polypeptides are also provided.

### CROSS-REFERENCE TO RELATED APPLICATIONS

This application is a continuation-in-part of U.S. patent application Ser. No. 09/693,542, filed Oct. 20, 2000, which is a continuation-in-part of U.S. patent application Ser. No. 09/566,617, filed May 8, 2000, which is a continuation-in-part of U.S. patent application Ser. No. 09/295,028, filed Apr. 20, 1999, which is a continuation in part of U.S. patent application Ser. No. 09/159,469, filed Sep. 23, 1998, which is a continuation in part of U.S. patent application Ser. No. 09/106,582, filed Jun. 29, 1998, which is a continuation-in-part of U.S. patent application Ser. No. 08/975,762, filed Nov. 20, 1997, which is a continuation-in-part of U.S. patent application Ser. No. 08/821,324, filed Mar. 21, 1997.

### TECHNICAL FIELD

The present invention relates generally to the detection and treatment of Ehrlichia infection. In particular, the invention is related to polypeptides comprising an Ehrlichia antigen and the use of such polypeptides for the serodiagnosis and treatment of Human granulocytic ehrlichiosis (HGE).

### BACKGROUND OF THE INVENTION

Human granulocytic ehrlichiosis (HGE) is an illness caused by a rodent bacterium which is generally transmitted to humans by the same tick that is responsible for the transmission of Lyme disease and babesiosis, thereby leading to the possibility of co-infection with Lyme disease, babesiosis and HGE from a single tick bite. The bacterium that causes HGE (referred to herein as Ehrlichia phagocytophila) is believed to be quite widespread in parts of the northeastern United States and has been detected in parts of Europe. While the number of reported cases of HGE infection is increasing rapidly, infection with Ehrlichia, including co-infection with Lyme disease, often remains undetected for extended periods of time. HGE is a potentially fatal disease, with the risk of death increasing if appropriate treatment is delayed beyond the first few days after symptoms occur. In contrast, deaths from Lyme disease and babesiosis are relatively rare.

The preferred treatments for HGE, Lyme disease and babesiosis are different, with penicillin's, such as doxycycline and amoxicillin, being most effective in treating Lyme disease, anti-malarial drugs being preferred for the treatment of babesiosis and tetracycline being preferred for the treatment of ehrlichiosis. Accurate and early diagnosis of Ehrlichia infection is thus critical but methods currently employed for diagnosis are problematic.

All three tick-borne illnesses share the same flu-like symptoms of muscle aches, fever, headaches and fatigue, thus making clinical diagnosis difficult. Microscopic analysis of blood samples may provide false-negative results when patients are first seen in the clinic. The only tests currently available for the diagnosis of HGE infection are indirect fluorescent antibody staining methods for total immunoglobulins to Ehrlichia causative agents and polymerase chain reaction (PCR) amplification tests. Such methods are time-consuming, labor-intensive and expensive. There thus remains a need in the art for improved methods for the detection of Ehrlichia infection, particularly as related to HGE. The present invention fulfills this need and further provides other related advantages.

### SUMMARY OF THE INVENTION

The present invention provides compositions and methods for the diagnosis and treatment of Ehrlichia infection and, in particular, for the diagnosis and treatment of HGE. In one aspect, polypeptides are provided comprising an immunogenic portion of an Ehrlichia antigen, particularly one associated with HGE, or a variant of such an antigen. In one embodiment, the antigen comprises an amino acid sequence encoded by a polynucleotide selected from the group consisting of (a) SEQ ID NO: 1-7, 15-22, 31, 34, 36, 39-49, 86, 88 and 94-98; (b) the complements of said sequences; (c) sequences that hybridize to a sequence of (a) or (b) under moderately stringent conditions; (d) sequences that have either 75% or 90% identity to a sequence of (a) or (b), determined as described below; and (e) degenerate variants of SEQ ID NO: 1-7, 15-22, 31, 34, 36, 39-49, 86, 88 and 94-98.

In another aspect, the present invention provides an antigenic epitope of an Ehrlichia antigen comprising an amino acid sequence selected from the group consisting of sequences recited in SEQ ID NO: 30 and 51, together with polypeptides comprising at least two such antigenic epitopes, the epitopes being contiguous.

In a related aspect, polynucleotides encoding the above polypeptides, recombinant expression vectors comprising one or more such polynucleotides and host cells transformed or transfected with such expression vectors are also provided.

In another aspect, the present invention provides fusion proteins comprising either a first and a second inventive polypeptide, a first and a second inventive antigenic epitope, or, alternatively, an inventive polypeptide and an inventive antigenic epitope. In specific embodiments, a fusion protein comprising an amino acid sequence provided in SEQ ID NO: 85, 92 or 93 is provided.

In further aspects of the subject invention, methods and diagnostic kits are provided for detecting Ehrlichia infection in a patient. In one embodiment, the method comprises: (a) contacting a biological sample with at least one of the above polypeptides, antigenic epitopes or fusion proteins; and (b) detecting in the sample the presence of antibodies that bind to the polypeptide, antigenic epitope or fusion protein, thereby detecting Ehrlichia infection in the biological sample. Suitable biological samples include whole blood, sputum, serum, plasma, saliva, cerebrospinal fluid and urine. The diagnostic kits comprise one or more of the above polypeptides, antigenic epitopes or fusion proteins in combination with a detection reagent.

The present invention also provides methods for detecting Ehrlichia infection comprising: (a) obtaining a biological sample from a patient; (b) contacting the sample with at least two oligonucleotide primers in a polymerase chain reaction, at least one of the oligonucleotide primers being specific for a polynucleotide encoding the above polypeptides; and (c) detecting in the sample a polynucleotide that amplifies in the presence of the oligonucleotide primers. In one embodiment, the oligonucleotide primer comprises at least about 10 contiguous nucleotides of a polynucleotide encoding the above polypeptides.

In a further aspect, the present invention provides a method for detecting Ehrlichia infection in a patient comprising: (a) obtaining a biological sample from the patient; (b) contacting the sample with an oligonucleotide probe specific for a polynucleotide encoding the above polypeptides; and (c) detecting in the sample a polynucleotide that hybridizes to the oligonucleotide probe. In one embodiment, the oligonucleotide probe comprises at least about 15 contiguous nucleotides of a polynucleotide encoding one of the above polypeptides.

In yet another aspect, the present invention provides antibodies, both polyclonal and monoclonal, that bind to the polypeptides described above, as well as methods for their use in the detection of Ehrlichia infection.

In further aspects, the present invention provides methods for detecting either Ehrlichia infection, Lyme disease or B. microti infection in a patient. Such inventive methods comprise: (a) obtaining a biological sample from the patient; (b) contacting the sample with (i) at least one of the inventive polypeptides, antigenic epitopes or fusion proteins, (ii) a known Lyme disease antigen, and (iii) a known B. microti antigen; and (c) detecting in the sample the presence of antibodies that bind to the inventive polypeptide, antigenic epitope or fusion protein, the known Lyme disease antigen or the known B. microti antigen, thereby detecting either Ehrlichia infection, Lyme disease or B. microti infection in the patient.

Within other aspects, the present invention provides pharmaceutical compositions that comprise one or more of the above polypeptides or antigenic epitopes, or polynucleotides encoding such polypeptides, and a physiologically acceptable carrier. The invention also provides immunogenic compositions comprising one or more of the inventive polypeptides or antigenic epitopes and an immunostimulant, together with immunogenic compositions comprising one or more polynucleotides encoding such polypeptides and an immunostimulant.

In yet another aspect, methods are provided for inducing protective immunity in a patient, comprising administering to a patient an effective amount of one or more of the above pharmaceutical compositions or immunogenic compositions.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 shows the results of Western blot analysis of representative Ehrlichia antigens of the present invention.

FIGS. 2A and B show the reactivity of purified recombinant Ehrlichia antigens HGE-1 and HGE-3, respectively, with sera from HGE-infected patients, babesiosis-infected patients, Lyme-disease infected patients and normal donors as determined by Western blot analysis.

SEQ ID NO: 1 is the determined DNA sequence of HGE-1.

SEQ ID NO: 2 is the determined DNA sequence of HGE-3.

SEQ ID NO: 3 is the determined DNA sequence of HGE-6.

SEQ ID NO: 4 is the determined 5′ DNA sequence of HGE-7.

SEQ ID NO: 5 is the determined DNA sequence of HGE-12.

SEQ ID NO: 6 is the determined DNA sequence of HGE-23.

SEQ ID NO: 7 is the determined DNA sequence of HGE-24.

SEQ ID NO: 8 is the predicted protein sequence of HGE-1.

SEQ ID NO: 9 is the predicted protein sequence of HGE-3.

SEQ ID NO: 10 is the predicted protein sequence of HGE-6.

SEQ ID NO: 11 is the predicted protein sequence of HGE-7.

SEQ ID NO: 12 is the predicted protein sequence of HGE-12.

SEQ ID NO: 13 is the predicted protein sequence of HGE-23.

SEQ ID NO: 14 is the predicted protein sequence of HGE-24.

SEQ ID NO: 15 is the determined 5′ DNA sequence of HGE-2.

SEQ ID NO: 16 is the determined DNA sequence of HGE-9.

SEQ ID NO: 17 is the determined DNA sequence of HGE-14.

SEQ ID NO: 18 is the determined 5′ DNA sequence of HGE-15.

SEQ ID NO: 19 is the determined 5′ DNA sequence of HGE-16.

SEQ ID NO: 20 is the determined 5′ DNA sequence of HGE-17.

SEQ ID NO: 21 is the determined 5′ DNA sequence of HGE-18.

SEQ ID NO: 22 is the determined 5′ DNA sequence of HGE-25.

SEQ ID NO: 23 is the predicted protein sequence of HGE-2.

SEQ ID NO: 24 is the predicted protein sequence of HGE-9.

SEQ ID NO: 25 is the predicted protein sequence of HGE-14.

SEQ ID NO: 26 is the predicted protein sequence of HGE-18.

SEQ ID NO: 27 is the predicted protein sequence from the reverse complement of HGE-14.

SEQ ID NO: 28 is the predicted protein sequence from the reverse complement of HGE-15.

SEQ ID NO: 29 is the predicted protein sequence from the reverse complement of HGE-18.

SEQ ID NO: 30 is a 41 amino acid repeat sequence from HGE-14.

SEQ ID NO: 31 is the determined DNA sequence of HGE-11.

SEQ ID NO: 32 is the predicted protein sequence of HGE-11.

SEQ ID NO: 33 is the predicted protein sequence from the reverse complement of HGE-11.

SEQ ID NO: 34 is the determined DNA sequence of HGE-13.

SEQ ID NO: 35 is the predicted protein sequence of HGE-13.

SEQ ID NO: 36 is the determined DNA sequence of HGE-8.

SEQ ID NO: 37 is the predicted protein sequence of HGE-8.

SEQ ID NO: 38 is the predicted protein sequence from the reverse complement of HGE-8.

SEQ ID NO: 39 is the extended DNA sequence of HGE-2.

SEQ ID NO: 40 is the extended DNA sequence of HGE-7.

SEQ ID NO: 41 is the extended DNA sequence of HGE-8.

SEQ ID NO: 42 is the extended DNA sequence of HGE-11.

SEQ ID NO: 43 is the extended DNA sequence of HGE-14.

SEQ ID NO: 44 is the extended DNA sequence of HGE-15.

SEQ ID NO: 45 is the extended DNA sequence of HGE-16.

SEQ ID NO: 46 is the extended DNA sequence of HGE-18.

SEQ ID NO: 47 is the extended DNA sequence of HGE-23.

SEQ ID NO: 48 is the extended DNA sequence of HGE-25.

SEQ ID NO: 49 is the determined 3′ DNA sequence of HGE-17.

SEQ ID NO: 50 is the extended predicted protein sequence of HGE-2.

SEQ ID NO: 51 is the amino acid repeat sequence of HGE-2.

SEQ ID NO: 52 is a second predicted protein sequence of HGE-7.

SEQ ID NO: 53 is a third predicted protein sequence of HGE-7.

SEQ ID NO: 54 is a second predicted protein sequence of HGE-8.

SEQ ID NO: 55 is a third predicted protein sequence of HGE-8.

SEQ ID NO: 56 is a fourth predicted protein sequence of HGE-8.

SEQ ID NO: 57 is a fifth predicted protein sequence of HGE-8.

SEQ ID NO: 58 is a second predicted protein sequence of HGE-11.

SEQ ID NO: 59 is a third predicted protein sequence of HGE-11.

SEQ ID NO: 60 is a second predicted protein sequence from the reverse complement of HGE-14.

SEQ ID NO: 61 is a third predicted protein sequence from the reverse complement of HGE-14.

SEQ ID NO: 62 is a first predicted protein sequence of HGE-15.

SEQ ID NO: 63 is a second predicted protein sequence of HGE-15.

SEQ ID NO: 64 is a second predicted protein sequence from the reverse complement of HGE-15.

SEQ ID NO: 65 is the predicted protein sequence of HGE-16.

SEQ ID NO: 66 is a first predicted protein sequence from the reverse complement of HGE-17.

SEQ ID NO: 67 is a second predicted protein sequence from the reverse complement of HGE-17.

SEQ ID NO: 68 is a second predicted protein sequence from the reverse complement of HGE-18.

SEQ ID NO: 69 is a third predicted protein sequence from the reverse complement of HGE-18.

SEQ ID NO: 70 is a fourth predicted protein sequence from the reverse complement of HGE-18.

SEQ ID NO: 71 is a second predicted protein sequence of HGE-23.

SEQ ID NO: 72 is a third predicted protein sequence of HGE-23.

SEQ ID NO: 73 is the predicted protein sequence of HGE-25.

SEQ ID NO: 74-79 are primers used in the preparation of a fusion protein containing HGE-9, HGE-3 and HGE-1.

SEQ ID NO: 80-83 are primers used in the preparation of a fusion protein containing HGE-3 and HGE-1 (referred to as ErF-1).

SEQ ID NO: 84 is the DNA sequence of the fusion ErF-1.

SEQ ID NO: 85 is the amino acid sequence of the fusion protein ErF-1.

SEQ ID NO: 86 is the full-length cDNA sequence for HGE-17.

SEQ ID NO: 87 is the amino acid sequence for HGE-17.

SEQ ID NO: 88 is a corrected cDNA sequence for HGE-14.

SEQ ID NO: 89 is the amino acid encoded by SEQ ID NO: 88.

SEQ ID NO: 90 is the DNA sequence of the coding region for a fusion protein containing HGE-9 with HGE-3 (known as ERF-2).

SEQ ID NO: 91 is the DNA sequence of the coding region for a fusion protein containing HGE-9 with HGE-1 (known as ERF-3).

SEQ ID NO: 92 is the amino acid sequence of ERF-2.

SEQ ID NO: 93 is the amino acid sequence of ERF-3.

SEQ ID NO: 94 is a corrected cDNA sequence for HGE-1.

SEQ ID NO: 95 is the reverse complement of SEQ ID NO: 39.

SEQ ID NO: 96 is the reverse complement of SEQ ID NO: 43.

SEQ ID NO: 97 is the reverse complement of SEQ ID NO: 44 with 314 bp of 5′ sequence removed.

SEQ ID NO: 98 is the reverse complement of SEQ ID NO: 86.

SEQ ID NO: 99 is the amino acid sequence of the variable region of the HGE-1 protein.

SEQ ID NO: 100 is the amino acid sequence of the variable region of the HGE-3 protein.

SEQ ID NO: 101 is the amino acid sequence of the variable region of the HGE-6 protein.

SEQ ID NO: 102 is the amino acid sequence of the variable region of a first HGE-7 protein.

SEQ ID NO: 103 is the amino acid sequence of the variable region of a second HGE-7 protein.

SEQ ID NO: 104 is the amino acid sequence of the variable region of the HGE-12 protein.

SEQ ID NO: 105 is the amino acid sequence of the variable region of a first HGE-23 protein.

SEQ ID NO: 106 is the amino acid sequence of the variable region of a second HGE-23 protein.

SEQ ID NO: 107 is the amino acid sequence of the variable region of a third HGE-23 protein.

SEQ ID NO: 108 is the amino acid sequence of the variable region of the HGE-34 protein.

### DETAILED DESCRIPTION OF THE INVENTION

As noted above, the present invention is generally directed to compositions and methods for the diagnosis and treatment of Ehrlichia infection, in particular HGE. In one aspect, the compositions of the subject invention include polypeptides that comprise at least one immunogenic portion of an Ehrlichia antigen, or a variant of such an antigen.

As used herein, the term “polypeptide” encompasses amino acid chains of any length, including fall length proteins (i.e., antigens), wherein the amino acid residues are linked by covalent peptide bonds. Thus, a polypeptide comprising an immunogenic portion of one of the above antigens may consist entirely of the immunogenic portion, or may contain additional sequences. The additional sequences may be derived from the native Ehrlichia antigen or may be heterologous, and such sequences may (but need not) be immunogenic.

An “immunogenic portion” of an antigen is a portion that is capable of reacting with sera obtained from an Ehrlichia-infected individual (i.e., generates an absorbance reading with sera from infected individuals that is at least three standard deviations above the absorbance obtained with sera from uninfected individuals, in a representative ELISA assay described herein). Such immunogenic portions generally comprise at least about 5 amino acid residues, more preferably at least about 10, and most preferably at least about 20 amino acid residues. Methods for preparing and identifying immunogenic portions of antigens of known sequence are well known in the art and include those summarized in Paul, Fundamental Immunology, 3^{rd} ed., Raven Press, 1993, pp. 243-247. Polypeptides comprising at least an immunogenic portion of one or more Ehrlichia antigens as described herein may generally be used, alone or in combination, to detect HGE infection in a patient.

The compositions and methods of the present invention also encompass variants of the above polypeptides and polynucleotides. Such variants include, but are not limited to, naturally occurring allelic variants of the inventive sequences.

A polypeptide “variant,” as used herein, is a polypeptide that differs from a native protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants encompassed by the present invention include those exhibiting at least about 70%, 75%, 80%, 85%, 90%, 91%, 92%, 93%, 94%, 95%, 96%, 97%, 98%, or 99% identity (determined as described below) to the polypeptides disclosed herein.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

Polynucleotides may comprise a native sequence (i.e., an endogenous sequence that encodes a protein or a portion thereof) or may comprise a variant of such a sequence, or a biological or antigenic functional equivalent of such a sequence. Polynucleotide variants may contain one or more substitutions, additions, deletions and/or insertions, as further described below, preferably such that the immunogenicity of the encoded polypeptide, relative to the native protein, is not diminished. The effect on the immunogenicity of the encoded polypeptide may generally be assessed as described herein. As used herein, the term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins —Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy-the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Preferred examples of algorithms that are suitable for determining percentage sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always>0) and N (penalty score for mismatching residues; always<0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

The present invention thus encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% sequence identity compared to a polynucleotide or polypeptide sequence of this invention using the methods described herein, (e.g., BLAST analysis using standard parameters, as described above). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5 ×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

In general, Ehrlichia antigens, and polynucleotides encoding such antigens, may be prepared using any of a variety of procedures. For example, polynucleotides encoding Ehrlichia antigens may be isolated from an Ehrlichia genomic or cDNA expression library by screening with sera from HGE-infected individuals as described below in Example 1, and sequenced using techniques well known to those of skill in the art. Polynucleotides encoding Ehrlichia antigens may also be isolated by screening an appropriate Ehrlichia expression library with anti-sera (e.g., rabbit) raised specifically against Ehrlichia antigens.

Antigens may be induced from such clones and evaluated for a desired property, such as the ability to react with sera obtained from an HGE-infected individual as described herein. Alternatively, antigens may be produced recombinantly, as described below, by inserting a polynucleotide that encodes the antigen into an expression vector and expressing the antigen in an appropriate host. Antigens may be sequenced, either partially or fully, using, for example, traditional Edman chemistry. See Edman and Berg, Eur. J. Biochem. 80:116-132, 1967.

Polynucleotides encoding antigens may also be obtained by screening an appropriate Ehrlichia cDNA or genomic DNA library for polynucleotides that hybridize to degenerate oligonucleotides derived from partial amino acid sequences of isolated antigens. Degenerate oligonucleotide sequences for use in such a screen may be designed and synthesized, and the screen may be performed, as described (for example) in Sambrook et al., Molecular Cloning: A Laboratory Manual, Cold Spring Harbor Laboratories, Cold Spring Harbor, N.Y. (and references cited therein). Polymerase chain reaction (PCR) may also be employed, using the above oligonucleotides in methods well known in the art, to isolate a nucleic acid probe from a cDNA or genomic library. The library screen may then be performed using the isolated probe.

Synthetic polypeptides having fewer than about 100 amino acids, and generally fewer than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division, Foster City, Calif., and may be operated according to the manufacturer's instructions.

Immunogenic portions of Ehrlichia antigens may be prepared and identified using well known techniques, such as those summarized in Paul, Fundamental Immunology, 3d ed., Raven Press, 1993, pp. 243-247 and references cited therein. Such techniques include screening polypeptide portions of the native antigen for immunogenic properties. The representative ELISAs described herein may generally be employed in these screens. An immunogenic portion of a polypeptide is a portion that, within such representative assays, generates a signal in such assays that is substantially similar to that generated by the full length antigen. In other words, an immunogenic portion of an Ehrlichia antigen generates at least about 20%, and preferably about 100%, of the signal induced by the fall length antigen in a model ELISA as described herein.

Portions and other variants of Ehrlichia antigens may be generated by synthetic or recombinant means. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Recombinant polypeptides containing portions and/or variants of a native antigen may be readily prepared from a polynucleotide encoding the polypeptide using a variety of techniques well known to those of ordinary skill in the art. For example, supernatants from suitable host/vector systems which secrete recombinant protein into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant protein.

Any of a variety of expression vectors known to those of ordinary skill in the art may be employed to express recombinant polypeptides as described herein. Expression may be achieved in any appropriate host cell that has been transformed or transfected with an expression vector containing a polynucleotide that encodes a recombinant polypeptide. Suitable host cells include prokaryotes, yeast and higher eukaryotic cells. Preferably, the host cells employed are E. coli, yeast or a mammalian cell line, such as COS or CHO. The polynucleotides expressed in this manner may encode naturally occurring antigens, portions of naturally occurring antigens, or other variants thereof.

In another aspect, the present invention provides antigenic epitopes of an Ehrlichia antigen or epitope repeat sequences, as well as polypeptides comprising at least two such contiguous antigenic epitopes. As used herein, an “epitope” is a portion of an antigen that reacts with sera from Ehrlichia-infected individuals (i.e. an epitope is specifically bound by one or more antibodies present in such sera). As discussed above, epitopes of the antigens described in the present application may be generally identified using techniques well known to those of skill in the art.

In specific embodiments, antigenic epitopes of the present invention comprise an amino acid sequence selected from the group consisting of sequence recited in SEQ ID NO: 30 and 51. As discussed in more detail below, antigenic epitopes provided herein may be employed in the diagnosis and treatment of Ehrlichia infection, either alone or in combination with other Ehrlichia antigens or antigenic epitopes. Antigenic epitopes and polypeptides comprising such epitopes may be prepared by synthetic means, as described generally above and in detail in Example 3.

In general, regardless of the method of preparation, the polypeptides and antigenic epitopes disclosed herein are prepared in an isolated, substantially pure, form. Preferably, the polypeptides and antigenic epitopes are at least about 80% pure, more preferably at least about 90% pure and most preferably at least about 99% pure.

In a further aspect, the present invention provides fusion proteins comprising either a first and a second inventive polypeptide, a first and a second inventive antigenic epitope, or an inventive polypeptide and an antigenic epitope of the present invention, together with variants of such fusion proteins. The fusion proteins of the present invention may also include a linker peptide between the polypeptides or antigenic epitopes.

A polynucleotide encoding a fusion protein of the present invention may be constructed using known recombinant DNA techniques to assemble separate DNA sequences encoding, for example, the first and second polypeptides, into an appropriate expression vector. The 3′ end of a DNA sequence encoding the first polypeptide is ligated, with or without a peptide linker, to the 5′ end of a DNA sequence encoding the second polypeptide so that the reading frames of the sequences are in phase to permit mRNA translation of the two DNA sequences into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8562, 1986; U.S. Pat. Nos. 4,935,233 and 4,751,180. The linker sequence may be from 1 to about 50 amino acids in length. As an alternative to the use of a peptide linker sequence (when desired), one can utilize non-essential N-terminal amino acid regions (when present) on the first and second polypeptides to separate the functional domains and prevent steric hindrance.

In another aspect, the present invention provides methods for using the polypeptides, fusion proteins and antigenic epitopes described above to diagnose Ehrlichia infection, in particular HGE. In this aspect, methods are provided for detecting Ehrlichia infection in a biological sample, using one or more of the above polypeptides, fusion proteins and antigenic epitopes, either alone or in combination. For clarity, the term “polypeptide” will be used when describing specific embodiments of the inventive diagnostic methods. However, it will be clear to one of skill in the art that the antigenic epitopes and fusion proteins of the present invention may also be employed in such methods.

As used herein, a “biological sample” is any antibody-containing sample obtained from a patient. Preferably, the sample is whole blood, sputum, serum, plasma, saliva, cerebrospinal fluid or urine. More preferably, the sample is a blood, serum or plasma sample obtained from a patient. The polypeptides are used in an assay, as described below, to determine the presence or absence of antibodies to the polypeptide(s) in the sample, relative to a predetermined cut-off value. The presence of such antibodies indicates previous sensitization to Ehrlichia antigens which may be indicative of HGE.

In embodiments in which more than one polypeptide is employed, the polypeptides used are preferably complementary (i.e., one component polypeptide will tend to detect infection in samples where the infection would not be detected by another component polypeptide). Complementary polypeptides may generally be identified by using each polypeptide individually to evaluate serum samples obtained from a series of patients known to be infected with HGE. After determining which samples test positive (as described below) with each polypeptide, combinations of two or more polypeptides may be formulated that are capable of detecting infection in most, or all, of the samples tested.

A variety of assay formats are known to those of ordinary skill in the art for using one or more polypeptides to detect antibodies in a sample. See, e.g., Harlow and Lane, Antibodies: A Laboratory Manual, Cold Spring Harbor Laboratory, 1988, which is incorporated herein by reference. In a preferred embodiment, the assay involves the use of polypeptide immobilized on a solid support to bind to and remove the antibody from the sample. The bound antibody may then be detected using a detection reagent that contains a reporter group. Suitable detection reagents include antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the antigen may be attached. For example, the solid support may be a test well in a microtiter plate, or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptides may be bound to the solid support using a variety of techniques known to those of ordinary skill in the art. In the context of the present invention, the term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide ranging from about 10 ng to about 1 μg, and preferably about 100 ng, is sufficient to bind an adequate amount of antigen.

Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to supports having an appropriate polymer coating using benzoquinone or by condensation of an aldehyde group on the support with an amine and an active hydrogen on the polypeptide (see, e.g., Pierce Immunotechnology Catalog and Handbook, 1991, at A12-A13).

In certain embodiments, the assay is an enzyme linked immunosorbent assay (ELISA). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin (BSA) or Tween 20™ (Sigma Chemical Co., St. Louis, Mo.) may be employed. The immobilized polypeptide is then incubated with the sample, and antibody is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody within an HGE-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., and Pierce, Rockford, Ill. ).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-Ehrlichia antibodies in the sample, the signal detected from the reporter group that remains bound to the solid support is generally compared to a signal that corresponds to a predetermined cut-off value. In one preferred embodiment, the cut-off value is the average mean signal obtained when the immobilized antigen is incubated with samples from an uninfected patient. In general, a sample generating a signal that is three standard deviations above the predetermined cut-off value is considered positive for HGE. In an alternate preferred embodiment, the cut-off value is determined using a Receiver Operator Curve, according to the method of Sackett et al., Clinical Epidemiology: A Basic Science for Clinical Medicine, Little Brown and Co., 1985, pp. 106-107. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for HGE.

In a related embodiment, the assay is performed in a rapid flow-through or strip test format, wherein the antigen is immobilized on a membrane, such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of anti-Ehrlichia antibodies in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when the biological sample contains a level of antibodies that would be sufficient to generate a positive signal in an ELISA, as discussed above. Preferably, the amount of polypeptide immobilized on the membrane ranges from about 25 ng to about 1 μg, and more preferably from about 50 ng to about 500 ng. Such tests can typically be performed with a very small amount (e.g., one drop) of patient serum or blood.

Of course, numerous other assay protocols exist that are suitable for use with the polypeptides and antigenic epitopes of the present invention. The above descriptions are intended to be exemplary only.

The inventive polypeptides may be employed in combination with known Lyme disease and/or B. microti antigens to diagnose the presence of either Ehrlichia infection, Lyme disease and/or B. microti infection, using either the assay formats described herein or other assay protocols. One example of an alternative assay protocol which may be usefully employed in such methods is a Western blot, wherein the proteins present in a biological sample are separated on a gel, prior to exposure to a binding agent. Such techniques are well known to those of skill in the art. Lyme disease antigens which may be usefully employed in such methods are well known to those of skill in the art and include, for example, those described by Magnarelli, L. et al. (J. Clin. Microbiol., 1996 34:237-240), Magnarelli, L. (Rheum. Dis. Clin. North Am., 1989, 15:735-745) and Cutler, S. J. (J. Clin. Pathol., 1989, 42:869-871). B. microti antigens which may be usefully employed in the inventive methods include those described in U.S. patent application Ser. No. 08/845,258, filed Apr. 24, 1997, the disclosure of which is hereby incorporated by reference.

In yet another aspect, the present invention provides antibodies to the polypeptides and antigenic epitopes of the present invention. Antibodies may be prepared by any of a variety of techniques known to those of ordinary skill in the art. See, e.g., Harlow and Lane, Antibodies: A Laboratory Manual, Cold Spring Harbor Laboratory, Cold Spring Harbor, N.Y., 1988. In one such technique, an immunogen comprising the antigenic polypeptide or epitope is initially injected into any of a wide variety of mammals (e.g., mice, rats, rabbits, sheep and goats). The polypeptides and antigenic epitopes of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide or antigenic epitope may then be purified from such antisera by, for example, affinity chromatography using the polypeptide coupled to a suitable solid support.

Monoclonal antibodies specific for the antigenic polypeptide or epitope of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide or antigenic epitope of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide or antigenic epitope. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides or antigenic epitopes of this invention may be used in the purification process in, for example, an affinity chromatography step.

Antibodies may be used in diagnostic tests to detect the presence of Ehrlichia antigens using assays similar to those detailed above and other techniques well known to those of skill in the art, thereby providing a method for detecting Ehrlichia infection in a patient.

The presence of HGE infection may also, or alternatively, be detected based on the level of mRNA encoding an HGE-specific protein in a biological sample, such as whole blood, serum, plasma, saliva, cerebrospinal fluid and urine. For example, at least two oligonucleotide primers may be employed in a polymerase chain reaction (PCR) based assay to amplify a portion of an HGE-specific polynucleotide derived from a biological sample, wherein at least one of the oligonucleotide primers is specific for (i.e., hybridizes to) a polynucleotide encoding the HGE protein. The amplified polynucleotide is then separated and detected using techniques well known in the art, such as gel electrophoresis. Similarly, oligonucleotide probes that specifically hybridize to a polynucleotide encoding an HGE protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a sequence that is complementary to a portion of a polynucleotide encoding an HGE protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule that is complementary to a polynucleotide disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an uninfected individual. The amplification reaction may be performed on several dilutions of cDNA spanning two orders of magnitude. A two-fold or greater increase in expression in several dilutions of the test patient sample as compared to the same dilutions of the non-infected sample is typically considered positive.

In another aspect, the present invention provides methods for using one or more of the above polypeptides, antigenic epitopes or fusion proteins (or polynucleotides encoding such polypeptides) to induce protective immunity against Ehrlichia infection in a patient. As used herein, a “patient” refers to any warm-blooded animal, preferably a human. A patient may be afflicted with a disease, or may be free of detectable disease and/or infection. In other words, protective immunity may be induced to prevent or treat Ehrlichia infection, specifically HGE.

In this aspect, the polypeptide, antigenic epitope, fusion protein or polynucleotide is generally present within a pharmaceutical composition or a vaccine (also referred to as an immunogenic composition). Pharmaceutical compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant or a liposome (into which the polypeptide is incorporated). Such pharmaceutical and immunogenic compositions may also contain other Ehrlichia antigens, either incorporated into a combination polypeptide or present as a separate polypeptide.

Alternatively, an immunogenic composition may contain DNA encoding one or more polypeptides, antigenic epitopes or fusion proteins as described above, such that the polypeptide is generated in situ. In such immunogenic compositions, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), virus. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In a related aspect, a DNA vaccine, or immunogenic composition, as described above may be administered simultaneously with or sequentially to either a polypeptide of the present invention or a known Ehrlichia antigen. For example, administration of DNA encoding a polypeptide of the present invention, either “naked” or in a delivery system as described above, may be followed by administration of an antigen in order to enhance the protective immune effect of the immunogenic composition.

Routes and frequency of administration, as well as dosage, will vary from individual to individual. In general, the pharmaceutical compositions and immunogenic compositions may be administered by injection (e.g., intracutaneous, intramuscular, intravenous or subcutaneous), intranasally (e.g., by aspiration) or orally. Between 1 and 3 doses may be administered for a 1-36 week period. Preferably, 3 doses are administered, at intervals of 3-4 months, and booster vaccinations may be given periodically thereafter. Alternate protocols may be appropriate for individual patients. A suitable dose is an amount of polypeptide or DNA that, when administered as described above, is capable of raising an immune response in an immunized patient sufficient to protect the patient from HGE for at least 1-2 years. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL.

While any suitable carrier known to those of ordinary skill in the art may be employed in the compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of this invention to enhance the immune response. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants. In certain embodiments, the inventive immunogenic compositions include an adjuvant capable of eliciting a predominantly Th-1 type response. Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corp. (Hamilton, Mont.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555 and WP 99/33488. Immunostimulatory DNA sequences are also described, for example, by Sato et al., Science 273:352, 1996. Another preferred adjuvant is a saponin, preferably QS21 (Aquila, United States), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

The following Examples are offered by way of illustration and not by way of limitation.

### EXAMPLE 1

Isolation of DNA Sequences Encoding Ehrlichia Antigens

This example illustrates the preparation of DNA sequences encoding Ehrlichia antigens by screening an Ehrlichia genomic expression library with sera obtained from mice infected with the HGE agent.

Ehrlichia genomic DNA was isolated from infected human HL60 cells and sheared by sonication. The resulting randomly sheared DNA was used to construct an Ehrlichia genomic expression library (approximately 0.5 -4.0 kbp inserts) with EcoRI adaptors and a Lambda ZAP II/EcoRI/CIAP vector (Stratagene, La Jolla, Calif.). The unamplified library (6.5×10⁶/ml) was screened with an E. coli lysate-absorbed Ehrlichia mouse serum pool, as described in Sambrook et al., Molecular Cloning: A Laboratory Manual, Cold Spring Harbor Laboratories, Cold Spring Harbor, N.Y., 1989. Positive plaques were visualized and purified with goat-anti-mouse alkaline phosphatase. Phagemid from the plaques was rescued and DNA sequence for positive clones was obtained using forward, reverse, and specific internal primers on a Perkin Elmer/Applied Biosystems Inc. Automated Sequencer Model 373A (Foster City, Calif.).

Of the eighteen antigens isolated using this technique, seven (hereinafter referred to as HGE-1, HGE-3, HGE-6, HGE-7, HGE-12, HGE-23 and HGE-24) were found to be related. The determined DNA sequences for HGE-1, HGE-3, HGE-6, HGE-12, HGE-23 and HGE-24 are shown in SEQ ID NO: 1-3 and 5-7, respectively, with the 5′ DNA sequence for HGE-7 being provided in SEQ ID NO: 4. The deduced amino acid sequences for HGE-1, HGE-3, HGE-6, HGE-7, HGE-12, HGE-23 and HGE-24 are provided in SEQ ID NO: 8-14, respectively. Comparison of these sequences with known sequences in the gene bank using the DNA STAR system, revealed some degree of homology to the Anaplasma marginale major surface protein.

Of the remaining eleven isolated antigens, no significant homologies were found to HGE-2, HGE-9, HGE-14, HGE-15, HGE-16, HGE-17, HGE-18 and HGE-25. The determined full-length cDNA sequences for HGE-9 and HGE-14 are provided in SEQ ID NO: 16 and 17, respectively, with the determined 5′ DNA sequences for HGE-2, HGE-15, HGE-16, HGE-17, HGE-18 and HGE-25 being shown in SEQ ID NO: 15, and 18-22, respectively. The corresponding predicted amino acid sequences for HGE-2, HGE-9, HGE-14 and HGE-18 are provided in SEQ ID NO: 23-26, respectively. The reverse complements of HGE-14, HGE-15 and HGE-18 were found to contain open reading frames which encode the amino acid sequences shown in SEQ ID NO: 27, 28 and 29, respectively. The predicted amino acid sequence from the reverse complement strand of HGE-14 (SEQ ID NO: 27) was found to contain a 41 amino acid repeat, provided in SEQ ID NO: 30. The full-length cDNA sequence for HGE-14 provided in SEQ ID NO: 17 was subsequently found to contain minor sequencing errors. A corrected full-length cDNA sequence for HGE-14 is provided in SEQ ID NO: 88, with the corresponding amino acid sequence being provided in SEQ ID NO: 89. The cDNA sequence of SEQ ID NO: 88 differs from that of SEQ ID NO: 17 by 2 nucleotides.

The determined DNA sequence for the isolated antigen HGE-11 is provided in SEQ ID NO: 31, with the predicted amino acid sequences being provided in SEQ ID NO: 32 and 33. Comparison of these sequences with known sequence in the gene bank, revealed some homology between the amino acid sequence of SEQ ID NO: 32 and that of bacterial DNA-directed RNA polymerase beta subunit rpoB (Monastyrskaya, G. S. et al., 1990, Bioorg. Khim. 6:1106-1109), and further between the amino acid sequence of SEQ ID NO: 33 and that of bacterial DNA-directed RNA polymerase beta' subunit rpoC (Borodin A. M. et al, 1988 Bioorg. Khim. 14:1179-1182).

The determined 5′ DNA sequence for the antigen HGE-13 is provided in SEQ ID NO: 34. The opposite strand for HGE-13 was found to contain an open reading frame which encodes the amino acid sequence provided in SEQ ID NO: 35. This sequence was found to have some homology to bacterial 2,3-biphosphoglycerate-independent phosphoglycerate mutase (Leyva-Vazquez, M. A. and Setlow, P., 1994 J. Bacteriol. 176:3903-3910).

The determined partial nucleotide sequence for the isolated antigen HGE-8 (SEQ ID NO: 36) was found to include, on the reverse complement of the 5′ end, two open reading frames encoding the amino acid sequences provided in SEQ ID NO: 37 and 38. The amino acid sequences of SEQ ID NO: 37 and 38 were found to show some homology to prokaryotic and eukaryotic dihydrolipamide succinyltransferase (Fleischmann R. D. et al, 1995 Science 269:496-512) and methionine aminopeptidase (Chang, Y. H., 1992 J. Biol. Chem. 267:8007-8011), respectively.

Subsequent studies resulted in the determination of extended DNA sequences for HGE-2, HGE-7, HGE-8, HGE-11, HGE-14, HGE-15, HGE-16, HGE-18, HGE-23 and HGE-25 (SEQ ID NO: 39-48, respectively) and in the determination of the 3′ sequence for HGE-17 (SEQ ID NO: 49). The complement of the extended HGE-2 DNA sequence was found to contain an open reading frame which encodes for a 61.4 kDa protein (SEQ ID NO: 50) having three copies of a 125 amino acid repeat (SEQ ID NO: 51). The extended DNA sequence of HGE-7 was found to contain two open reading frames encoding for the amino acid sequences shown in SEQ ID NO: 52 and 53. The extended DNA sequence of HGE-8 was found to contain four open reading frames encoding the proteins of SEQ ID NO: 54-57. Each of these four proteins was found to show some similarity to known proteins, however, to the best of the inventors' knowledge, none have previously been identified in Ehrlichia.

The extended DNA sequence of HGE-11 was found to contain two open reading frames encoding the amino acid sequences provided in SEQ ID NO: 58 and 59. These two proteins were found to show some homology to the bacterial DNA-directed RNA polymerase beta subunits rpoB and rpo C, respectively. The reverse complement of the extended DNA sequence of HGE-14 was found to contain two open reading frames, with one encoding the amino acid sequence provided in SEQ ID NO: 60. The second open reading frame encodes the amino acid sequence provided in SEQ ID NO: 61, which contains the amino acid sequence provided in SEQ ID NO: 27. The extended DNA sequence of HGE-15 was found to contain two open reading frames encoding for the sequences provided in SEQ ID NO: 62 and 63, with a third open reading frame encoding the sequence of SEQ ID NO: 64 being located on the reverse complement. The extended DNA sequence of HGE-16 was found to contain an open reading frame encoding the amino acid sequence of SEQ ID NO: 65. The reverse complement of the 3′ DNA sequence of HGE-17 was found to contain two open reading frames encoding the amino acid sequences of SEQ ID NO: 66 and 67.

The reverse complement of the extended DNA sequence of HGE-18 was found to contain three open reading frames encoding the amino acid sequences of SEQ ID NO: 68-70. The sequence of SEQ ID NO: 70 was found to show some homology to bacterial DNA helicase. The extended DNA sequence of HGE-23 was found to contain two open reading frames encoding for the sequences of SEQ ID NO:71 and 72. Both of these sequences, together with those of SEQ ID NO:52 and 53, were found to share some homology with the Anaplasma marginale major surface protein. The predicted amino acid sequence encoded by the extended DNA sequence of HGE-25 is provided in SEQ ID NO:73. This sequence was found to show some similarity to that of SEQ ID) NO:64 (HGE-15). No significant homologies were found to the amino acid sequences of HGE-2, HGE-14, HGE-15, HGE-16, HGE-17 and HGE-25 (SEQ ID NO: 50, 60-67 and 73).

Using standard full-length cloning techniques, the full-length cDNA sequence for HGE-17 was isolated. This sequence is provided in SEQ ID NO: 86, with the corresponding amino acid sequence being provided in SEQ ID NO: 87. These sequences were found to show some homology to the known sequences for ankyrin.

Further review of the cDNA sequence of HGE-1 provided in SEQ ID NO: 1, revealed that 265 bp of the 3′ sequence represents a second insert in the cloned DNA. The cDNA sequence of HGE-1 without this insert is provided in SEQ ID NO: 94. SEQ ID NO: 95 represents the reverse complement of the cloned cDNA sequence of HGE-2 provided in SEQ ID NO: 39. Similarly, SEQ ID NO: 96 represents the reverse complement of the cloned sequence of HGE-14 provided in SEQ ID NO: 43. The sequence of SEQ ID NO: 97 represents the reverse complement of the cloned cDNA sequence of HGE-15 (SEQ ID NO: 44) with 314 bp of sequence representing a second insert being removed from the 5′ end. SEQ ID NO: 98 represents the reverse complement of the cloned cDNA sequence of HGE-17 (SEQ ID NO: 86) with 2401 bp removed from the 3′ end of the reverse complement.

Alignment of the polypeptide sequence from HGE-1, HGE-3, HGE-6, HGE-7, HGE-12, HGE-23 and HGE-34 resulted in a pattern of conserved and variable regions. The predicted amino termini are well conserved except for variability at the extreme amino end due to variations in ORF size. This conserved region is followed by a variable region of approximately 71 to 91 amino acid residues and then a second conserved region near the carboxy termini. The amino acid sequences of the variable regions of HGE-1, HGE-3, HGE-6, the first and second protein sequences of HGE-7, HGE-12, the first, second and third protein sequences of HGE-23, and HGE-34 are provided in SEQ ID NO: 99-108, respectively.

### EXAMPLE 2

Use of Representative Antigens for Serodiagnosis of HGE Infection

The diagnostic properties of representative Ehrlichia antigens were determined by Western blot analysis as follows.

Antigens were induced as pBluescript SK-constructs (Stratagene), with 2 mM IPTG for three hours (T3), after which the resulting proteins from time 0 (T0) and T3 were separated by SDS-PAGE on 15% gels. Separated proteins were then transferred to nitrocellulose and blocked for 1 hr in 1% BSA in 0.1% Tween 20™/PBS. Blots were then washed 3 times in 0.1% Tween 20™/PBS and incubated with either an HGE patient serum pool (1:200) or an Ehrlichia-infected mouse serum pool for a period of 2 hours. After washing in 0.1% Tween 20™/PBS 3 times, blots were incubated with a second antibody (goat-anti-human IgG conjugated to alkaline phosphatase (AP) or goat-anti-mouse IgG-AP, respectively) for 1 hour. Immunocomplexes were visualized with NBT/BCIP (Gibco BRL) after washing with Tween 20™/PBS three times and AP buffer (100 mM Tris-HCl, 100 mM NaCl, 5 mM MgCl₂, pH 9.5) two times.

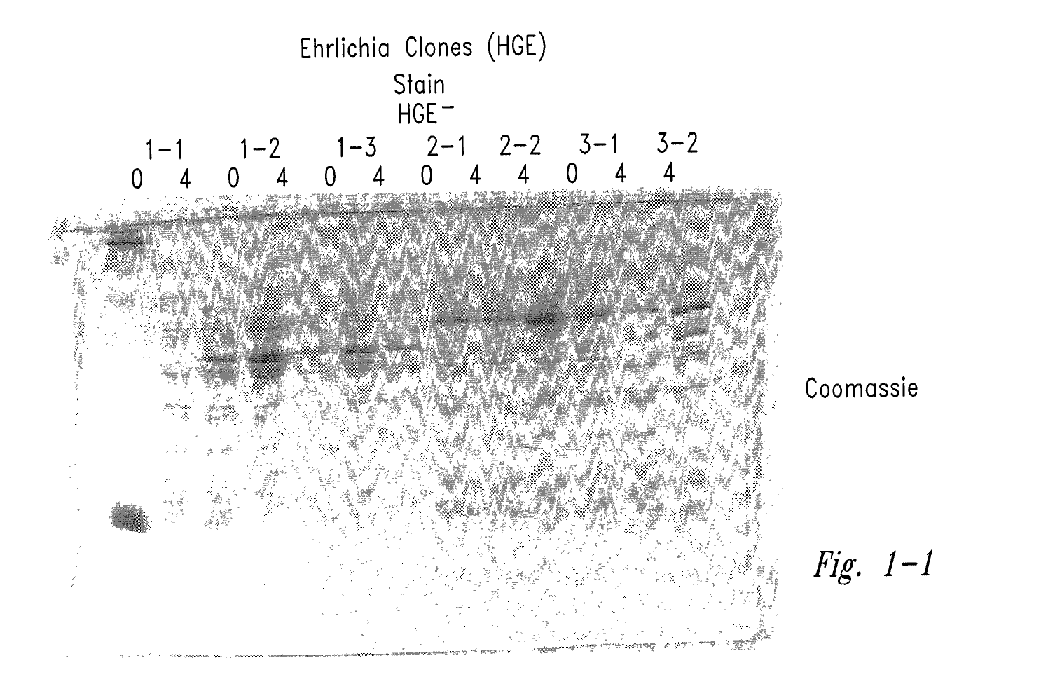

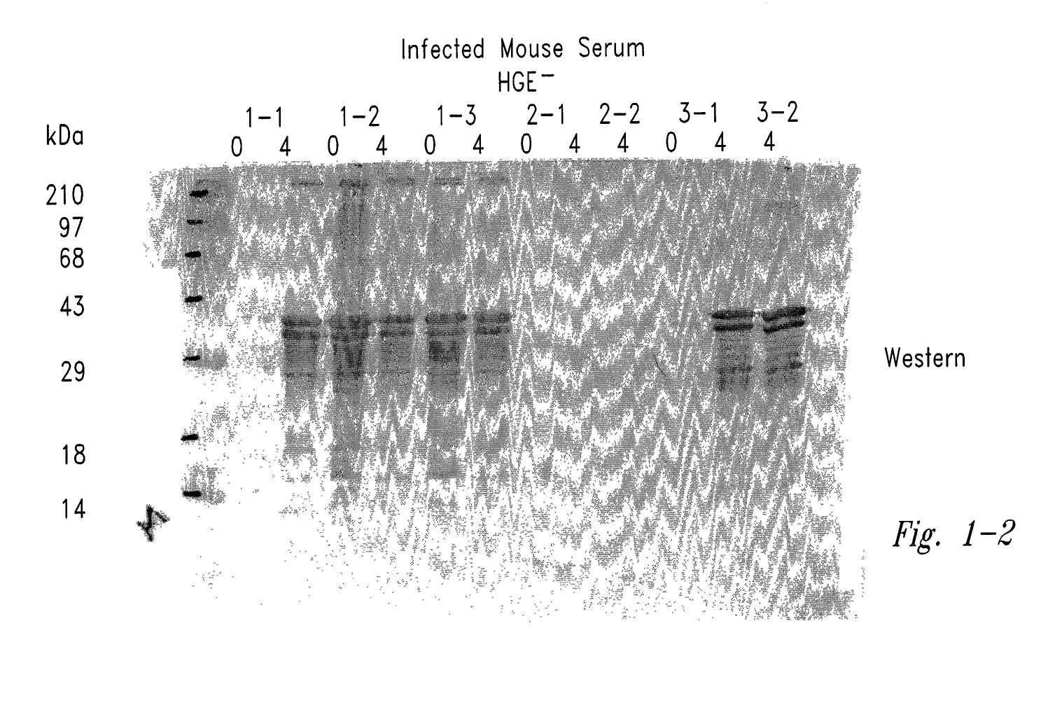

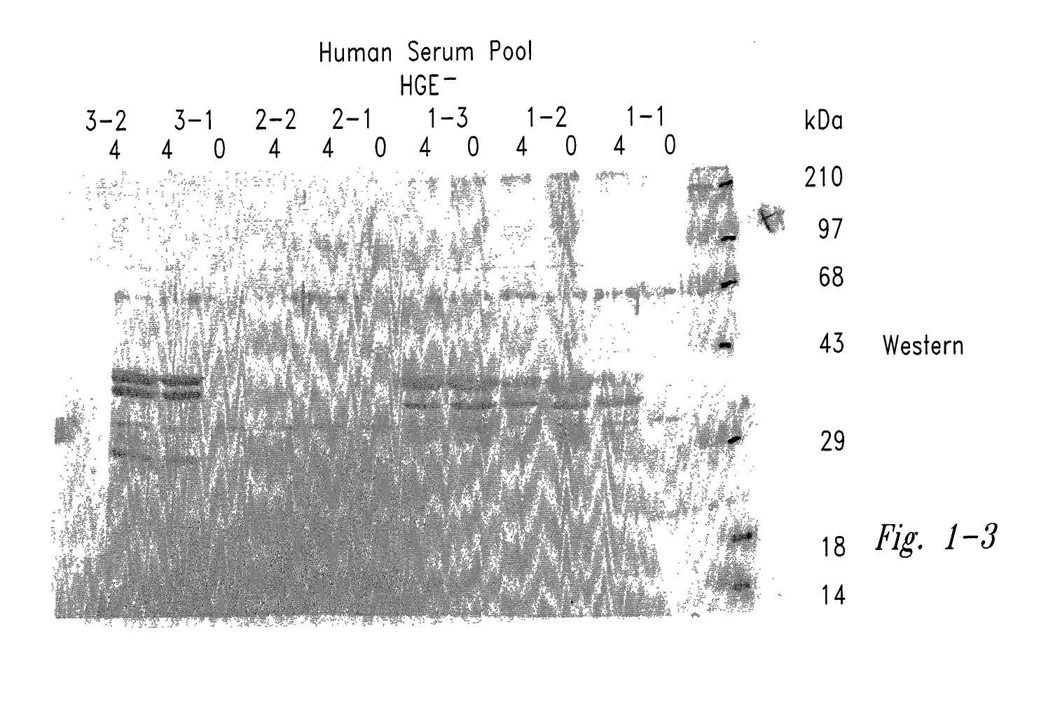

As shown in FIG. 1, resulting bands of reactivity with serum antibody were seen at 37 kDa for HGE-1 and HGE-3 for both the mouse serum pool and the human serum pool. Protein size standards, in kDa (Gibco BRL, Gaithersburg, Md.), are shown to the left of the blots.

Western blots were performed on partially purified HGE-1 and HGE-3 recombinant antigen with a series of patient sera from HGE patients, patients with Lyme disease, babesiosis patients or from normal donors. Specifically, purified antigen (4 μg) was separated by SDS-PAGE on 12% gels. Protein was then transferred to nitrocellulose membrane for immunoblot analysis. The membrane was first blocked with PBS containing 1% Tween 20™ for 2 hours. Membranes were then cut into strips and incubated with individual sera (1/500) for two hours. The strips were washed 3 times in PBS/0.1% Tween 20™ containing 0.5 M NaCl prior to incubating with Protein A-horseradish peroxidase conjugate (1/20,000) in PBS/0.1% Tween 20™/0.5 M NaCl for 45 minutes. After further washing three times in PBS/0.1% Tween 20™/0.5 M NaCl, ECL chemiluminescent substrate (Amersham, Arlington Heights, Ill.) was added for 1 min. Strips were then reassembled and exposed to Hyperfilm ECL (Amersham) for 5-30 seconds.

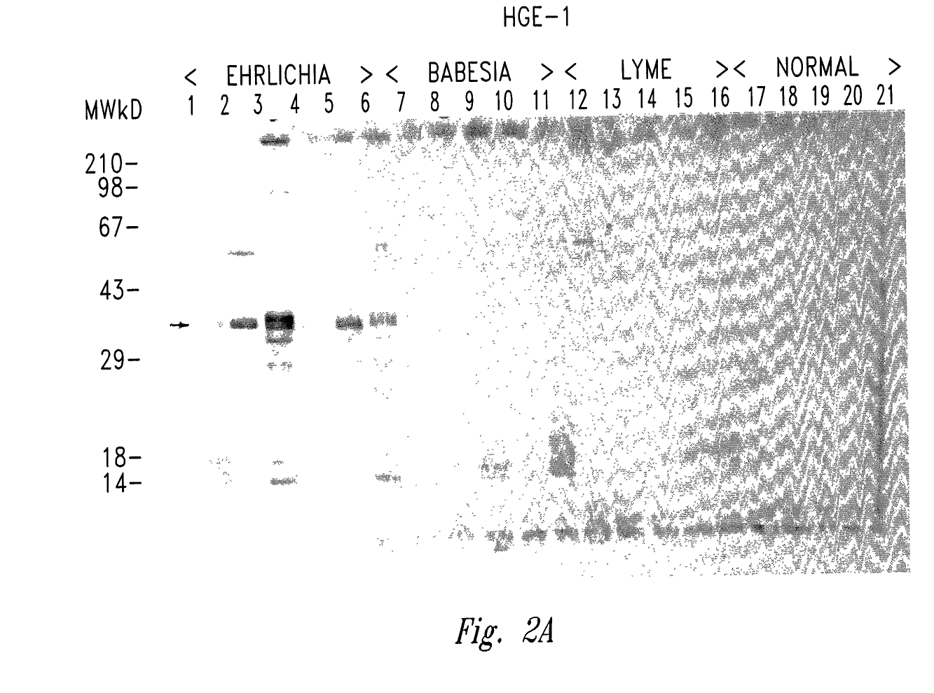

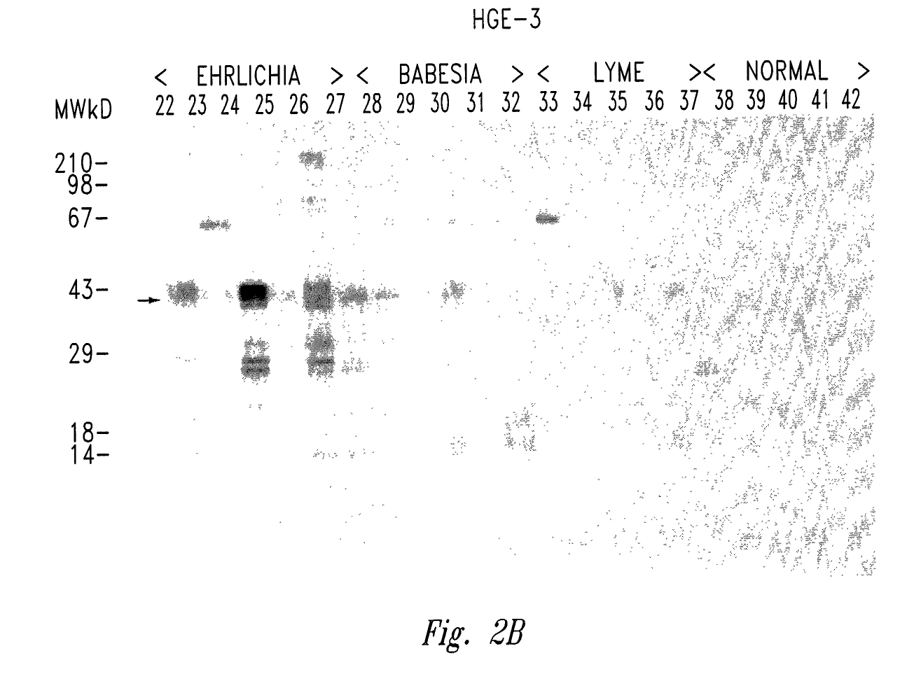

Lanes 1-6 of FIG. 2A show the reactivity of purified recombinant HGE-1 (MW 37 kD) with sera from six HGE-infected patients, of which all were clearly positive. In contrast, no immunoreactivity with HGE-1 was seen with sera from patients with either babesiosis (lanes 7-11), or Lyme disease (lanes 12-16), or with sera from normal individuals (lanes 17-21). As shown in FIG. 2B, HGE-3 (MW 37 kD) was found to react with sera from all six HGE patients (lanes 22-27), while cross-reactivity was seen with sera from two of the five babesiosis patients and weak cross-reactivity was seen with sera from two of the five Lyme disease patients. This apparent cross-reactivity may represent the ability of the antigen HGE-3 to detect low antibody titer in patients co-infected with HGE. No immunoreactivity of HGE-3 was seen with sera from normal patients.

Table 1 provides representative data from studies of the reactivity of HGE-1, HGE-3 and HGE-9 with both IgG and IgM in sera from patients with acute (A) or convalescent (C) HGE, determined as described above. The antibody titer for each patient, as determined by immunofluorescence, is also provided.

TABLE 1

| Patient | HGE | IgG | | | IgM | | |
|---|---|---|---|---|---|---|---|
| ID | titer | HGE-1 | HGE-3 | HGE-9 | HGE-1 | HGE-3 | HGE-9 |
| 1 (A) | 128 | 0.346 | 0.154 | 0.423 | 0.067 | 0.028 | 0.022 |
| 2 (A) | 1024 | 1.539 | 1.839 | 0.893 | 2.75 | 3.256 | 1.795 |
| 3 (A) | <16 | 0.412 | 0.16 | 0.659 | 0.043 | 0.088 | 0.047 |
| 4 (A) | <16 | 0.436 | 0.072 | 0.472 | 0.017 | 0.032 | 0.064 |
| 5 (C) | 256 | 0.322 | 0.595 | 0.694 | 0.229 | 0.345 | 0.269 |
| 6 (A) | 512 | 1.509 | 2.042 | 1.241 | 0.721 | 0.695 | 0.313 |
| 7 (C) | 512 | 0.508 | 1.019 | 0.777 | 0.45 | 0.777 | 0.29 |
| 8 (C) | 128 | 0.635 | 0.979 | 1.684 | 0.729 | 2.079 | 0.729 |
| 9 (C) | 256 | 0.408 | 0.74 | 0.679 | 0.052 | 0.11 | 0.062 |
| 10 (A) | 64 | 0.579 | 0.133 | 0.239 | −0.002 | 0.015 | 0.126 |
| 11 (A) | 256 | 0.13 | 0.066 | 1.002 | −0.018 | 0.003 | 0.047 |
| 12 (A) | 16 | 0.347 | 0.249 | 0.727 | 0.135 | 0.071 | 0.113 |
| 14 (A) | 1024 | 2.39 | 3.456 | 2.635 | 1.395 | 1.52 | 0.55 |

These results indicate that HGE-9 is able to complement the serological reactivity of HGE-1 and HGE-3, leading to increased sensitivity in the serodiagnosis of HGE-infection in convalescent and acute patient sera, as shown, for example, with patients 5, 8, 11 and 12 in Table 1.

### EXAMPLE 3

Preparation and Characterization of Ehrlichia Fusion Proteins

A fusion protein containing the Ehrlichia antigens HGE-9, HGE-3 and HGE-1 is prepared as follows.

Each of the DNA constructs HGE-9, HGE-3 and HGE-1 are modified by PCR in order to facilitate their fusion and the subsequent expression of the fusion protein. HGE-9, HGE-3 and HGE-1 DNA was used to perform PCR using the primers PDM-225 and PDM-226 (SEQ ID NO: 74 and 75), PDM-227 and PDM-228 (SEQ ID NO: 76 and 77), and PDM-229 and PDM-209 (SEQ ID NO: 78 and 79), respectively. In each case, the DNA amplification is performed using 10 μl of 10×Pfu buffer (Stratagene), 1 μl of 12.5 mM dNTPs, 2 μl each of the PCR primers at 10 μM concentration, 82 μl water, 2 μl Pfu DNA polymerase (Stratagene, La Jolla, Calif.) and 1 μl DNA at 110 ng/μl. Denaturation at 96° C. is performed for 2 min, followed by 40 cycles of 96° C. for 20 sec, 60° C. for 15 sec and 72° C. for 5 min, and lastly by 72° C. for 5 min.

The HGE-9 PCR fragment is cloned into pPDM HIS at the Eco 72 I sites along with a three-way ligation of HGE-3 or HGE-1 by cutting with Pvu I. HGE-3 is cloned into pPDM HIS which has been cut with Eco 72I/Xho I. HGE-1 is cloned into pPDM HIS which has been cut with Eco 72I/Eco RI. PCR is performed on the ligation mix of each fusion with the primers PDM-225, PDM-228 and PDM-209 using the conditions provided above. These PCR products are digested with Eco RI (for HGE-1) or Xho I (for HGE-3) and cloned into pPDM HIS which is digested with Eco RI (or Xho I) and Eco 721. The fusion construct is confirmed by DNA sequencing.

The expression construct is transformed to BLR pLys S E. coli (Novagen, Madison, Wis.) and grown overnight in LB broth with kanamycin (30 μg/ml) and chloramphenicol (34 μg/ml). This culture (12 ml) is used to inoculate 500 ml 2XYT with the same antibiotics and the culture is induced with IPTG. Four hours post-induction, the bacteria are harvested and sonicated in 20 mM Tris (8.0), 100 mM NaCl, 0.1% DOC, followed by centrifugation at 26,000×g. The resulting pellet is resuspended in 8 M urea, 20 mM Tris (8.0), 100 mM NaCl and bound to Ni NTA agarose resin (Qiagen, Chatsworth, Calif.). The column is washed several times with the above buffer then eluted with an imidazole gradient (50 mM, 100 mM, 500 mM imidazole is added to 8 M urea, 20 mM Tris (8.0), 100 mM NaCl). The eluates containing the protein of interest are then dialyzed against 10 mM Tris (8.0).

A fusion protein containing the Ehrlichia antigens HGE-3 and HGE-1, referred to as ErF-1, was prepared as follows.

HGE-3 and HGE-1 DNA was used to perform PCR using the primers PDM-263 and PDM-264 (SEQ ID NO: 80 and 81), and PDM-208 and PDM-265 (SEQ ID NO: 82 and 83), respectively. In both cases, the DNA amplification was performed using 10 μl of 10×Pfu buffer (Stratagene), 1 μl of 10 mM dNTPs, 2 μl each of the PCR primers at 10 μ M concentration, 83 μl water, 1.5 μl Pfu DNA polymerase (Stratagene, La Jolla, Calif.) and 1 μl DNA at 50 ng/μl. Denaturation at 96° C. was performed for 2 min, followed by 40 cycles of 96° C. for 20 sec, 60° C. for 15 sec and 72° C. for 3 min, and lastly by 72° C. for 4 min. The HGE-3 PCR product was digested with Eco 72I and Xho I, and cloned into pPDM His which had been digested with Eco 72I and Xho I. The HGE-1 PCR product was digested with ScaI, cloned into the above construct at the ScaI site, and screened for orientation. The fusion construct was confirmed by DNA sequencing. The determined DNA sequence of the fusion construct is provided in SEQ ID NO: 84.

The expression construct was transformed into BL21 pLys S E. coli (Novagen, Madison, Wis.) and grown overnight in LB broth with kanamycin (30 μg/ml) and chloramphenicol (34 μg/ml). This culture (12 ml) was used to inoculate 500 ml 2XYT with the same antibiotics and the culture was induced with IPTG. Four hours post-induction, the bacteria were harvested and sonicated in 20 mM Tris (8.0), 100 mM NaCl, 0.1% DOC, followed by centrifugation at 26,000×g. The protein came out in the inclusion body pellet. This pellet was washed three times with a 0.5% CHAPS wash in 20 mM Tris (8.0), 300 mM NaCl. The pellet was then solubilized in 6 M GuHCl, 20 mM Tris (9.0), 300 mM NaCl, 1% Triton X-100 and batch bound to Nickel NTA resin (Qiagen). The column was washed with 100 ml 8M urea, 20 mM Tris (9.0), 300 mM NaCl and 1% DOC. This wash was repeated but without DOC. The protein was eluted with 8 M urea, 20 mM Tris (9.0), 100 mM NaCl and 500 mM imidazole. In a second elution, the imidazole was increased to 1M. The elutions were run on a 4-20% SDS-PAGE gel and the fractions containing the protein of interest were pooled and dialyzed against 10 mM Tris (9.0). The amino acid sequence of the fusion protein ErF-1 is provided in SEQ ID NO: 85.

One of skill in the art will appreciate that the order of the individual antigens within the fusion protein may be changed and that comparable or enhanced activity could be expected provided each of the epitopes is still functionally available. In addition, truncated forms of the proteins containing active epitopes may be used in the construction of fusion proteins.

Table 2 provides representative data from studies of the reactivity of ErF-1, HGE-1 or HGE-3 with both IgG and IgM in sera from patients with acute (A) or convalescent (C) HGE, determined as described above in Example 2. The antibody titer for each patient, as determined by immunofluorescence, is also provided.

TABLE 2

| Patient | HGE | IgG | | | IgM | | |
|---|---|---|---|---|---|---|---|
| ID | titer | HGE-1 | HGE-3 | ErF-1 | HGE-1 | HGE-3 | ErF-1 |
| 1 (A) | 128 | 0.346 | 0.154 | 0.114 | 0.067 | 0.028 | 0.149 |
| 2 (A) | 1024 | 1.539 | 1.839 | 1.911 | 2.75 | 3.256 | 1.916 |
| 3 (A) | <16 | 0.412 | 0.16 | 0.096 | 0.043 | 0.088 | 0.104 |
| 4 (A) | <16 | 0.436 | 0.072 | 0.111 | 0.017 | 0.032 | 0.081 |
| 5 (C) | 256 | 0.322 | 0.595 | 0.713 | 0.229 | 0.345 | 0.190 |
| 6 (A) | 512 | 1.509 | 2.042 | 1.945 | 0.721 | 0.695 | 0.314 |
| 7 (C) | 512 | 0.508 | 1.019 | 1.206 | 0.45 | 0.777 | 0.361 |
| 8 (C) | 128 | 0.635 | 0.979 | 1.212 | 0.729 | 2.079 | 0.551 |
| 9 (C) | 256 | 0.408 | 0.74 | 0.767 | 0.052 | 0.11 | 0.157 |
| 10 (A) | 64 | 0.579 | 0.133 | 0.116 | −0.002 | 0.015 | 0.052 |
| 11 (A) | 256 | 0.13 | 0.066 | 0.039 | −0.018 | 0.003 | 0.022 |
| 12 (A) | 16 | 0.347 | 0.249 | 0.063 | 0.135 | 0.071 | 0.032 |
| 14 (A) | 1024 | 2.39 | 3.456 | 2.814 | 1.395 | 1.52 | 0.773 |

Table 3 shows the sensitivity and specificity of the reactivity of ErF-1, HGE-9, ErF-1 plus HGE-9, HGE-2, HGE-14, HGE-15 or HGE-17, with both IgG and IgM in sera from patients with acute (A) or convalescent (C) HGE, determined by ELISA as described above in Example 2. The theoretical results for a combination of ErF-1, HGE-9, HGE-2, HGE-14, HGE-15 and HGE-17 are also shown in Table 3. With the combination of all the recombinant antigens, 85.2% of the acute phase serum samples and 96.7% of the convalescent phase samples were detected, with a specificity of greater than 90%.

TABLE 3

| | Sensitivity | | | | | |
|---|---|---|---|---|---|---|
| | Acute | | Convalescent | | Specificity | |
| ErF-1 | | | | | | |
| IgG | 14/27 | (51.8%) | 25/27 | (92/6%) | 97.2% | (1/36) |
| IgM | 15/27 | (55.6%) | 23/27 | (85.2%) | 100% | (0/36) |
| IgG + IgM | 15/27 | (55.6%) | 25/27 | (92.6%) | 97.2% | (1/36) |
| HGE-9 | | | | | | |
| IgG | 18/27 | (66.7%) | 19/26 | (73.1%) | 97.3% | (1/37) |
| IgM | 12/27 | (44.4%) | 18/26 | (69.2%) | 100% | (0/37) |
| IgG + IgM | 20/27 | (74.1%) | 20/26 | (76.9%) | 97.3% | (1/37) |
| ErF-1 + HGE-9 | | | | | | |
| IgG | 19/27 | (70.4%) | 25/27 | (92.6%) | | |
| IgM | 16/27 | (59.2%) | 23/27 | (85.2%) | | |
| IgG + IgM | 21/27 | (77.8%) | 25/27 | (92.6%) | | |
| HGE-2 | | | | | | |
| IgG | 15/27 | (55.6%) | 21/26 | (80.8%) | 97.3% | (1/37) |
| IgM | 4/27 | (14.8%) | 3/26 | (11.5%) | 94.6% | (2/37) |
| IgG + IgM | 15/27 | (55.6%) | 21/26 | (80.8%) | 91.9% | (3/37) |
| HGE-14 | | | | | | |
| IgG | 13/27 | (48.1%) | 13/26 | (50.0%) | 96.8% | (1/31) |
| IgM | 8/27 | (29.6) | 7/26 | (26.9%) | 93.5% | (2/31) |
| IgG + IgM | 14/27 | (51.8%) | 13/26 | (50.0%) | 93.5% | (2/31) |
| HGE-15 | | | | | | |
| IgG | 12/27 | (44.4%) | 17/26 | (65.4%) | 97.3% | (1/37) |
| IgM | 12/27 | (44.4%) | 13/26 | (4850.0%%) | 97.3% | (1/37) |
| IgG + IgM | 13/27 | (48.1%) | 18/26 | (69.2%) | 94.6% | (2/37) |
| HGE-17 | | | | | | |
| IgG | 12/27 | (44.4%) | 13/26 | (50.0%) | 94.6% | (2/37) |
| IgM | 14/27 | (51.8%) | 14/26 | (53.8%) | 100% | (0/37) |
| IgG + IgM | 15/27 | (55.6%) | 18/26 | (69.2%) | 94.6% | (2/37) |
| ALL | | | | | | |
| ANTIGENS | | | | | | |
| IgG | 21/27 | (77.8%) | 26/27 | (96.3%) | | |
| IgM | 16/27 | (59.2%) | 22/27 | (81.5%) | | |
| IgG + IgM | 23/27 | (85.2%) | 26/27 | (96.2%) | | |

A fusion protein containing the Ehrlichia antigens HGE-9 and HGE-3, referred to as ErF-2, is prepared using the method described above for ERF-1, and employing the primers PDM-225 and PDM-226 (SEQ ID NO: 74 and 75, respectively) to PCR amplify HGE-9, and the primers PDM-227 and PDM-228 (SEQ ID NO: 76 and 77, respectively) to PCR amplify HGE-3. The DNA sequence of the coding region of ERF-2 is provided in SEQ ID NO: 90, with the amino acid sequence being provided in SEQ ID NO: 92.

A fusion protein containing the Ehrlichia antigens HGE-9 and HGE-1, referred to as ErF-3, is prepared using the method described above for ERF-1, and employing the primers PDM-225 and PDM-226 (SEQ ID NO: 74 and 75, respectively) to PCR amplify HGE-9, and the primers PDM-229 and PDM-209 (SEQ ID NO: 78 and 79, respectively) to PCR amplify HGE-1. The DNA sequence of the coding region of ERF-3 is provided in SEQ ID NO: 91, with the amino acid sequence being provided in SEQ ID NO: 93.

### EXAMPLE 4

Preparation of Synthetic Polypeptides

Polypeptides may be synthesized on a Millipore 9050 peptide synthesizer using FMOC chemistry with HPTU (O-Benzotriazole-N,N,N′,N′-tetramethyluronium hexafluorophosphate) activation. A Gly-Cys-Gly sequence may be attached to the amino terminus of the peptide to provide a method of conjugating or labeling of the peptide. Cleavage of the peptides from the solid support may be carried out using the following cleavage mixture: trifluoroacetic acid:ethanedithiol:thioanisole:water:phenol (40:1:2:2:3).

After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0-60% acetonitrile (containing 0.1% TFA) in water (containing 0.1% TFA) may be used to elute the peptides. Following lyophilization of the pure fractions, the peptides may be characterized using electrospray mass spectrometry and by amino acid analysis.

Although the present invention has been described in some detail by way of illustration and example for purposes of clarity of understanding, changes and modifications can be carried out without departing from the scope of the invention which is intended to be limited only by the scope of the appended claims.

1. An isolated polynucleotide comprising a sequence selected from the group consisting of:
(a) sequences provided in SEQ ID NO:1-7, 15-22, 31, 34, 36, 39-49, 86, 88 and 94-98;
(b) complements of the sequences provided in SEQ ID NO:1-7, 15-22, 31, 34, 36, 39-49, 86, 88 and 94-98;
(c) sequences that hybridize to a sequence provided in SEQ ID NO:1-7, 15-22, 31, 34, 36, 39-49, 86, 88 and 94-98, under moderately stringent conditions;
(d) sequences having at least 75% identity to a sequence of SEQ ID NO:1-7, 15-22, 31, 34, 36, 39-49, 86, 88 and 94-98;
(e) sequences having at least 90% identity to a sequence of SEQ ID NO: 1-7, 15-22, 31, 34, 36, 39-49, 86, 88 and 94-98; and
(f) degenerate variants of a sequence provided in SEQ ID NO:1-7, 15-22, 31, 34, 36, 39-49, 86, 88 and 94-98.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1; and
(c) sequences having at least 90% identity to a sequence encoded by a polynucleotide of claim 1.

3. The polypeptide of claim 2, wherein the polypeptide comprises an amino acid sequence selected from the group consisting of SEQ ID NO:8-14, 23-29, 32, 33, 35, 37, 38, 50, 52-73, 87 and 89.

4. An isolated antigenic epitope of an Ehrlichia antigen comprising an amino acid sequence selected from the group consisting of SEQ ID NO:30 and 51.

5. An isolated polypeptide comprising at least two antigenic epitopes according to claim 4.

6. A recombinant expression vector comprising a polynucleotide according to claim 1.

7. A host cell transformed with an expression vector according to claim 6.

8. A fusion protein comprising at least one polypeptide according to any one of claims 2 and 3.

9. The fusion protein of claim 8, wherein the fusion protein comprises an amino acid sequence selected from the group consisting of: SEQ ID NO:85, 92 and 93.

10. A fusion protein comprising at least one antigenic epitope according to claim 4.

11. A fusion protein comprising at least one polypeptide according to any one of claims 2 and 3 and at least one antigenic epitope according to claim 4.

12. A method for detecting Ehrlichia infection in a patient, comprising:
(a) obtaining a biological sample from the patient;
(b) contacting the biological sample with at least one polypeptide according to any one of claims 2 and 3; and
(c) detecting the presence of antibodies in the biological sample that bind to the polypeptide.

13. A method for detecting Ehrlichia infection in a patient, comprising:
(a) obtaining a biological sample from the patient;
(b) contacting the biological sample with at least one antigenic epitope according to claim 4; and
(c) detecting the presence of antibodies in the biological sample that bind to the antigenic epitope.

14. A method for detecting Ehrlichia infection in a patient, comprising:
(a) obtaining a biological sample from the patient;
(b) contacting the biological sample with a fusion protein according to any one of claims 8-11; and
(c) detecting the presence of antibodies in the biological sample that bind to the fusion protein.

15. A method for detecting Ehrlichia infection in a biological sample, comprising:
(a) contacting the biological sample with at least two oligonucleotide primers in a polymerase chain reaction, wherein at least one of the oligonucleotide primers is specific for a polynucleotide according to claim 1; and
(b) detecting in the biological sample a polynucleotide sequence that amplifies in the presence of the oligonucleotide primers, thereby detecting Ehrlichia infection.

16. A method for detecting Ehrlichia infection in a biological sample, comprising:
(a) contacting the sample with one or more oligonucleotide probes specific for a polynucleotide according to claim 1; and
(b) detecting in the sample a polynucleotide sequence that hybridizes to the oligonucleotide probe, thereby detecting Ehrlichia infection.

17. A method for detecting Ehrlichia infection in a biological sample, comprising:
(a) contacting the biological sample with a binding agent which is capable of binding to a polypeptide according to any one of claims 2 and 3; and
(b) detecting in the sample a polypeptide that binds to the binding agent, thereby detecting Ehrlichia infection in the biological sample.

18. A method of detecting Ehrlichia infection in a biological sample, comprising:
(a) contacting the biological sample with a binding agent which is capable of binding to a fusion protein according to any one of claims 8-11; and
(b) detecting in the sample a polypeptide that binds to the binding agent, thereby detecting Ehrlichia infection in the biological sample.

19. A method of detecting Ehrlichia infection in a biological sample, comprising:
(a) contacting the biological sample with a binding agent which is capable of binding to an antigenic epitope of claim 4; and
(b) detecting in the sample a polypeptide that binds to the binding agent, thereby detecting Ehrlichia infection in the biological sample.

20. A diagnostic kit comprising:
(a) at least one component selected from the group consisting of:
(i) polypeptides according to any one of claims 2 and 3;
(ii) antigenic epitopes according to claim 4; and
(iii) fusion proteins according to any one of claims 8-11; and
(b) a detection reagent.

21. A diagnostic kit comprising at least two oligonucleotide primers, at least one of the oligonucleotide primers being specific for a polynucleotide according to claim 1.

22. A diagnostic kit comprising at least one oligonucleotide probe, the oligonucleotide probe being specific for a polynucleotide according to claim 1.

23. An isolated antibody, or antigen-binding fragment thereof, that specifically binds to a polypeptide of claim 2.

24. An isolated antibody, or antigen-binding fragment thereof, that specifically binds an antigenic epitope according to claim 4.

25. A composition comprising a first component selected from the group consisting of physiologically acceptable carriers and immunostimulants, and a second component selected from the group consisting of:
(a) polypeptides according to any one of claims 2 and 3;
(b) polynucleotides according to claim 1;
(c) epitopes according to claim 4
(d) antibodies according to any one of claims 23 and 24; and
(e) fusion proteins according to any one of claims 8-11.

26. A method for stimulating an immune response in a patient, comprising administering to the patient a composition of claim 25.

27. A method for the treatment of Ehrlichia infection in a patient, comprising administering to the patient a composition of claim 25.

28. A method for detecting at least one disorder selected from the group consisting of Ehrlichia infection, Lyme disease and B. microti infection in a patient, the method comprising:
(a) obtaining a biological sample from the patient;
(b) contacting the biological sample with at least one polypeptide according to any one of claims 2 and 3, a Lyme disease antigen and a B. microti antigen; and
(c) detecting the presence of antibodies in the biological sample that bind to either the polypeptide, the Lyme disease antigen or the B. microti antigen.
